SEQUENCE LISTING (103 sequences)

SEQ ID NO: 1 - DNA, 39217 bp, Homo sapiens
aactttgctggctgctcctaggctcccggctcgccgccatcttgtattggggtttcattgttcccgctgggccgggcggtttagtgtaattgccgccggaggaggaggcggagtaacctctggtcagccgagaaaccccactatcctgtagccataaccgcttaaacgatttgggaggtagtgaagggcagggagctggacctggaggcgccgccgcgacagcagcagccatggaggacgagatgcccaagactctgtgagtctggggcagcgatgagggaggcgggatggtggtcgtcccggagggaaggcctcggccctgcgccgctcccagcctattgttctccgtggacaccgcgatatcgtggtttttgtttccttagcatgcacctggatgcttacaaccaagacccaatctgtgaggacgaggtgaagggagacggggctcaaccctctcaattaatcccttcactttagcaacaccattgcatgacctttgctctcgccacgggccctctgtggtcttagacctggagaaaagactttaattcagacctctttttagaactcgtcgaaccttccttttgtcctatgctttctttccttttcccgcttctccccctttttgattaacctcttcttatctttacagccgtctccctttaattttacttttgatttcctactgaaattcttcctttcattttcttctcggtgataagattttttttcctcctcgagggtgcttgtactcattttcagtgcagtaaactttattcttgcagttgccaattggaaaaaagccaagcatttttctttaacctctggctttccttctgtagcacccaccgaccaaaaagcctaaccagacaccccaaggatttggaaatctcacccagaaggaatatatcaattgaggggaaatggatttaatattatttttatagtcgtttactactgttgtgattgacatggtgttgaacgagcgggctgtaaatgccctaacatatttacagagaatttttagagatagcgttaaattaccggagcgtggatattttctttgtcgtgtggcttccccaactgcgttctttgtttttcgaggtattatcggatgagttgatcctgcctttgttggcatttctcggaagaagaaaagctcaatgagttcttgacatagctttttgtttttcttggatgacatagctttttgtttatattcttggaatactggcagtttgagagctccatgcattaaaaatgcaattttaggtttctctttgcagctctctaataaagttgctctgaagtacgatgtaattgtttttttacctccaaaatttttccacttttaagttgctatttcttgtctatattggaaatgtggttacacttgttttcttaatagattgtcagttaatctgttagtaaccttgaaatttcttttctctattcctgaggctcagaaagtatcttttcagtaggacgtagcaattagtgcatgacctttgttttaggaaagattgtatctgtagatgaatactccctaaaaaactactagcctcacggtgttggtggaatctcttccctcttgcctcttagctatgacagtcatcagcattttgggttaaggcactgctcgtgttttataatgtaaacttaatattctcagtattagtttgaaaatgtttaattttagaaactgcagaagtatctgaccacttaactttttaactttttaattttgtttttactttttaattttttgagatggagtctcactttgtcacccaggctgcaaggctagagtgcagtgatgcgatttcggctcactgcaacctccacctcctgggtttaaatgattctcctgcctcaccctcccaagtagctggcactacatgcgggcaccaccatgcccggctaatttttgtatttttagtagagatggggtttcaccatgtcggccaagctggtctcgaactcctgacctcaggtgatccgcacacctcggcctcccagagtgctgggattacaggtgtgagccactgcgcctggcctgtcctcaacttttaactattacatttattacttattttgccttgtatccctcactaatacaccccatccaagaaactcttcctcaaagtagtcatactgtaaaagtcttctcttgtttctacctccctcaacccaaactcccatttttctagtcaggcaaaccttgaccaaacatgagggtactaatccctaagttttcctaatagtgtttttaattttaatgtaagatttttgctttaatgaccctggaagtcagaggcttttaatatatgttcttagcagagttcttagcacattaaaaacacagcctctcaatatatgttcttagtgtggtccatggtaagtaatcaatacatttgtttaagaaactggtataattggctgggcacagtggctcacgcctataatcctaacactttggggggctgaggagggtggatcacttgagggcaggagttcaagaccagcctggccaacatggcgaaaccctgtctctactacaaatacaaaaattaactgagcatggtggcgcatgcctgtaatcccagctactcgggaggctgaggaaggagaatcgcttgaacccgggaggtggaggttgcagtgagccgaggttgcgccattgcactccagcctgtgcaacaggagggaggctccatctcaaaaaaaaaaaaaaaaaaaaaaaaaatctggtagaatttcaagcagtgaattaagattttactctttcttgaggataaaatagagtctgaatttttatcacccacatacctaaatggctcactggaaattccttcatttgctacatctgagtgttcggcatctccatgttaagttagagaataccagtatttgatttcttgtcaccaatactttggaatcatattctgagacaaacttcccgaaagtttatactgcaagattattttgaggagtattcaaggtacagttaataagttcaaacatttgtagtgagcatatgggtgatgtatactactcttctccatatataaatttgaaattttccacagtaaaaaaataaaaatacgtgtatagtgagtctgaattggccttggttactttaaaaatattcaacttgtgaactaaaagctacacactagactagaaaaacattgctggtgttagttgatgaaagttaattccctcaaatccagacaccttaatatcctttgtgtggtttactctcaaatgcatgagtgttctcttttgagataactatactgttattgtgcggctttcccatttgctaagatgcagatctgtaaattatagaccatctttaaaagcctgcagctggccaggcatggtggctcacacctgtaatcccagcactttgggaagccgaggtgagtggatcacctgaggtcagaagtttgagaccaacctgggcaacatggtgaaaccccatctctactaaaaatacaaaaattagctaggtgtggtgacacatgccagtagcctcagctactcgggactccggaggctcaggaaggagaatcgcccgggaggtggaggttgcagtgagccgagatcacgccactgcactccagcctggatgacagagcaagacggagcccaattaattgctatatactaatgagggccttgcctttcttctgctccatgctcatctttcattcttgttgtctactccactctccaaatagtattggccttgtaatttgtatgaaataaaatctcttgtattgagaatcagacctatctttttttttttttttttttttttgagacagagtctcgctctgtcacccaggctggaatgcagtggcccaatcttggctcactgcaagctctgccttccaggttcacgccattctcctgcctcagcctccggagtagctgggactgcaggcacccgccaccacgcctggctaattttttgtatttgtagtagagacacagtttcaccgtgttagccaggatggtctcgatctcctgacctcgcgatccgcctgcctcagcctcccaaagtgctgggattacaggcgacaatcagacctattttaaagattagattgaaggcaaggttggccaaaatgagatatttattagataatacttatgtttccaaataactgctagtaattaagaatatgctagaaacattacattcactcagtaatattgagagcatactacatggctgtccgtcttctgggaactggggatatggtagtacatataataggccttttacgaatttacataagtaattatgttagatggtaataagtgctatggagaaacataatgcagggtatgcatggtaaggaggaaggggtggatatttatagagaataataaaggaaggtcatcctttgataagtgacatttgaggaaaaaagtgaaggagatgagggagagccatgcttctatctgaaggaggagcaccctagaaagagctagtgcaaggggcccttgggggcaggtcatgctggttggttagcctgaagagagtaaatgtagtagaggagtaggaaatgaggtcagaatgataaataggaatgttaggggagatagaaaatgccagatcatacggatccttatgagggctatcttatctgagtaggaatggaagctgtaagtgggtttcacgcagagtagtggtatagcctatcttaaccttaaaactggcaaaagcaagtagcctagtcaggaagccaatccaggtatcagagaaagcagtgcctgtaattgtagttagtgctggtgaaagtgatgggaagtagttgaattatggatatgttttgaaaatagagcaacaggaattaccagtggcttagatgtggcatgtgagaatgatgtcaacaataaatggatttattttaaaatgtgtaatatcagttatctaaaatgttatagtttgttgcaaaatataagtaaaaatgaaaagtcttgaggatcgatatcccttaagatgtctacaggcatagtattgaatacatttgttcaagctggtttacctagtgaaggaggtcccaactagtaaggcagtttcagccatctcttgtaacagattattctttgtgatgggtatacatttgttaaataataataaggttaggatgctgatgtatagtatatacacaagcacttactattctacacaatttatttatttatttattttaattgagatagggtctcatcctgttgtccaggctctggcgcagtggtgtgatcacagctcactgtagcctcgacctctcaggctcaagggatcttcccacctcagcctcctgaatagctgggactataggcatgtgccaccatgtctggctaatttttaaaaatatttgtagagaagcgatctcactgtgttgcccaggctggtctccaactcctgggctcaagcgatcttcccaccttagcctcccaaagtgggattataggtgtgagccaccgtgcctggtgtttacacagtttcttcattagcaaaagacataactaatcttttccctctagggtaacttaagcaaaatgagagaattttctagcaaattgcaaatgttcagaagtcattctgagacagtacatttaactctttaacagatatttatagagcaataaatatcactgcaatgatattgcaatgttccaggcagtgttctagactagcagtccccagcctttttggcaccgagaccagttttgtggaagacaatttttccatgggcgggatggtttcaggatgaaactgttccacctcagagcatcaggcattagataaattctcataaggaacacacaacctagatcccttgcatgcccagttcacaaatgctgctgatcttacaggaggcagagctcaagcagtaatgctccctcccctgctgctcacctcctgctgtgtggcccagttcctaacaggccatggacgcataatggtccatggcctaggggggagttggggacccctgttctagatggtagataccaactaagaataaaaatgaaaacctcttatggaatttattattaggtaaaaaactattcatcgtttttactcaaattttcagagaccttatcaaattctgcctttttttttttttttttttttttttttgagactgagtctctgtcactcaggctggactgccgtggtgagatctcggctcactgcaacctccacttcccgggttcaggtgattctctcgcccaccatcacacttggctaattttttgtgtttttagtagaggcaagtttcaacatattggccaggctgatctcgaactcctgagctcaagtgatcccccacctcagcctcccaaagtgctgggattacaggcgtgagccaccgctcccagctaatcctgaaaatttcttataggcacaagagtcaggtgagagtagacacacaagagatgcagagatagtacagagttttgttttttgcgggggagggttttgcggaagaatggcatgataaccttcatctagcatcccttaatgttaccatcttatataaccgtggtataattatcaaaattaagaaattaacactataggctttattccaatttttccactgatatcctttttatattctaggatccaattcaggaaaataccacattgcatttcgtcatcgtctctttattctccttcattctgtgagaggtctttctgaccttaacacttttttttttttttagaggtaaggtctcactctggtgcccagactggagttcagtggcacaatcattgttcacttcagcttcccaagtagctggaactacaggtgcacaccaccacacccagctaattttttcttattttttgtagagacggtgcatatcactatgttgccctagctggtctcaaactactggcctcaagtgaatctctcacctcggcctcccaaagcactgggtttacaggtgtgagccaccacaccctaccgacactttggaagggtactgtttctcctgttctccaaaatcaccgtgacttcttttttcttaacttactctgttggttagaagtaagtcaccgaggctagctcacactcaggaagaggaagattaagttacacttcctggagggaggtatatccaaagaattcatagtcatgtaaaaccactagttcattggtcaaaacttttttttttctttttttaagacagagtctcgctctgttgccaggctggagtacagtggcatgatctcggctcactgcaacctctgcctcctgggttcaagcgattctcctgcctcggcctccctagtagctgaaactacaggcacacgccaccaagcccagctaattttttgtattttttagtagagacggggtttcactatgttggccaggatggtcttgatctcctgacctgtgatccgcctcggtctcccaaagtgctaggattacaggcgtgagccaccgcacctgtcccaaaaccaatttttaataaatattttagtggaggtactttgaggctattcagataccctgtttccccttttaaggtttcatcaactaatgtgagcttccatgggtggatcttcctttagaaattattactgtggtgttttaatggtgattttctatttctctcattctttctacatttattaattctgtaagaaaaaactattttcccctcatccgggatccatttaaacattattgattgtatttggttgtcagtgttttttatgtcctttttttttggtgggggggcagggagggcagtgtcttgctctgtcaccctggctggagtactgtggtgtaatatgagctcactgcaatgtccacctcccaagttcaagtgattcttgtgcctccccttcccaggtagctgggattacaggcatgcgccaccacacctggctgatttttgtatttattgtagtagagatgaggtttcaccatgttggccaggctggtctctaactcctggtctcaagtgaactgcctacctcggcctcccaaagtgctaggatttcaggcttgagccactgtgcccagctttctgtttcttttatttatttatatcttagaaatgaggtcttgctatgttcccaggctggactcaaactcctgggttcaagggatccacccacctcagcctctcaagtagctggggctgcgggcttgtgccaccactcccttttattcaagaacagtcatctgttgtttttgttttaggacaataattttttgaagagactgagttgtcttataaagtgtcccacatctggattcatggaattgttttatttgcctgatatcctataaacttaaatctaattttcagcttggttagatttaggttagacttttttatgtaaatatttcataggtgatactgtgtaattcatattatatatatgcagtatctcattggtcgttgcccatctataaaataattgaaaattttatcactgggtcatagtggtaacagccaaagctctcatttttaaggtaaggttttccctttgcaatttattaagtaatctatgtgataactttgacaccttgtgactatttccacaccaactgttcatgtagtagtttaagcattaattaaagatccttgcctgaaacagttatttcattagggtttacacattgttaatttaatactatctttctacatttattaactagcattctcctgtaaaggagaacttttctaaactagagctattagattacactatttagtgggagttcctacttaataggataaatgcttaattctgtctctttttcagaataaagacttggtgtagtaataactcccaatactggtaaattgctgttgggtaaatgttgctgttactttctcttttgagtaccattattttacctgatcgttaaatgtcatttgttattaagactcagcatttcttcctttgccatcttaaccttttccaaaggcttcgtgttaagcactcaatatgtgtttgttgaatggtagatggaaagaagaaattgattccacactttgtgagcatgtatgaatatttacttttctggggagaaagtccataccttgtatcaaattttctgaagggtacctgagagacaaatgagaaactaccattctggggagactgcattccctgattccagaactgataaacctctaactctgtaaaatgactagaggtaatctcatctcaaaatctcatttaaaaacttatttataaaccctttattggccgggcgcagtggctcacgcctgtaatccaaacactttgggaggccgaggcaagcagatcacgaggtcaggagatcgagaccatcctggctaacatggtgaaaccccatctctactaaaaatacaaaaaactagccgggcgtggtggcgggtgcctgtagtcccagctactcgggaggctgaggcaggagaatggcgtgaactcaggaggcggagcttgcagtgagccgagatcacgccactgcactccagcctgggcgacagagcaagactccgtctctaaataaatgaataaatagtaaaacctttattttctttttagagatagagttttgctctgtcacctgggatggagtgcagtggcataatcatagctcactgcagcctcttaactcctgggttcaagtgatcttcccacctcagcgtccgaagtagctgggaccatgccatgggacatagtgccaccatgtccgctaattttttatctttatttttattttttgtagagacgggggtctcgctgtattgactgggctgatcttgaactccttggctaaagtgatctttccaccttggcctccaaaagtgctgggattacagatgtgagccactgcccctaatctaaaccctttaaatgaagatcatcacagtcttatggtcttgtaatgcacgatttatatttttatattgataaccaactcataatctgtgatgtttctactccctttcacccctcttctactgatcagtaatgttaaatggaaggtttctcatcccactgtaagcattcaaaacaactgataatttacctataacttttcaaaaatgtgttcattgcataaccaaagtctacctaaattgaacaaataagggaaataatgttaatttaaaaattgggggtaccagcctggccaatatggtgaaaccccgtctctaccaaaaatataaaaaaattagctgggcgtggtggcgggcgcctgtagccccagctacgtgggtggctgaggcaggagaatcacttgaacctgggaggtggaggttgcaatgagccgagatcgcaccactgcactccagcctgggcgacagagagagactttgtccacaaaaaaaaaaaaaaaaaaaaaaaaaaacttgggggaacgtcaatgttcttagtagtagtaataccatagaagtagaaataattgcaatgccttttaggtttgatggtatacatttttttttttaattgagatggagtctcgctctgttgcccaagctggagtgcagtggcaccatctcggctcactgcaagttccgtctcccaggttcacgccgttctcctgcctcagcctcccgagtagccgggactacaggcgcccaccaccacgcccagctaattttttatatttttattagagacgaggtttcatcgtgttagccaggatggtcttgatctcctgacctcgtgatccgcccacctcggcctcccaaagtgctgggattacaggcatgagccaccgcacccagcctttttttttctttttaaagacagtcttgctccgtcacccaggctggagtgcagtggcgtgatcggggctcgttgcaacctctgcctcctgggttcaagcaattctcatgcctcagccgtcccccagtagctgggattacaggcaggcgccaccacgcccggctaatttttgtgtttttagtagagaaggggtttcactgtgttggccaggctggtatcaaactcctgcctcaagtgatcctctctcctcagcctcccaaagtgctgggattacaggtgtgagccacctcgcccagccattattgtataagatattttatataactaatgatgattcatttgagtgtttttaaagcttgtcttcctaaggtaagcctggaaacgtagaaacaagcaatcagtttgcaaaacgatgactaaaacagacaagcacatactagcttccccttcactagcaaggtgtaatatattttcttttttgagacatagtctcactctgttgcccaggctggagtgcagtggtgcaacctcagctaacccagcctctgcctcctgggttcaagtgattctcctgcctcagcctcccaagtagctgggtttacagatgtgcaccaccatgcctggctaatttttgtgtttttagtagagacagggtttcaccatgtttaccaggctggtctcaactcctggcctcaggtgatcttcccaccttggcttcccaaagtgctgggattacaggcgtgagccaccacacctagctaatatattttcaaataacatactttacatctgttttcaaagagattttgaagtgttggcaaacttttaaaagttactcccaaatattatagtttaaggcaaagatctgcctatttctcaagtttaatttttaatgaacaactttagaaaccgatggagaataagcaaattaaaaatggtaaatcaagggctgggcacaatgcctcatgcctgtaatctttaagattacactttgggaggccaaggcaggcagatcgcttgaacccaggagtttgagaccagctcagcaacatggtgaaacgccttctctacagaaactgtaaaaaaattagccagatatgatggcacacgcctgtggtcccagctacttgggaggctgaagtgggagaatcccttgtgcccaggaggtggagattgtagtgagccaagattgcgccactacactctagtctgggcaacccagaagaccttgtctgaaaaaaaaataaattgaggtaaattgagctgggtgtggtggttcctctagtccgttactcaggggaccaaggcagtaggtttgcttgaggccaggagtttgagagcagcctgagcaatatagccagactatttctcttaaaaaaaaaaaccgggtgcagtggctcacacctgtaatttcagcactttgggaggctgaagcaggcgggtcacctgaggtcaggagttggagaccagcctgaccaacatggagaaaccccgtctctactaaaaatacaaaattagctgggtgtggtcacgcatgcctgtaatcccagctactcggggaggctgaggcaggggaatcacttgaaccctggaggcggaggttgtggtgagccgagatcgtgccattgcactctatcctgggcaacaagagtgaaactctgtctcaaaaaaaaaaaaaggtaaattgtttctctttgtgtttcagatacgtcggtaacctttccagagatgtgacagaagctctaattctgcaactctttagccagattggaccttgtaaaaactgcaaaatgattatggatgtaagggtattttacttagttaactttattctttttttttttacacattacttaaagccattgcttggaaggaattcttagtttaccatggtctaaatatagtataaattattagtttactgtagtctaaatatagtcttagtttaccatattctaagttaatagggattttagtgaatttgtaatgaacctgttttgacagctgaatttaatttgttttattacatgatggaagatgacaatagaactagttatttctagatcagatgggaacatatatatgggctctttcaggtgcattgaggcatcagaaatgtttatatgtggtgtcaactggttggctaataaattcagctcttacaattaaattggaaaacaggtttaaaaataataggcatttggtacagtgaaagtatttttaactttatttttaaaggtcatcattcaagtttttcttgtatttaggcaattcctatttatgagacaatgtattaaacacacacacatacacacacacacacacacacacacacacactcattcatctaattggtgttaaccctgaatcataataggtataaaaagacatttagggaacatttaccccatttccctggagtctttgtctcctggttggggggaaaagactttaaatttttttttgagacggagttttgctcttgttgcccaggctggaatacaatggtgcgatctcggctcgctgcaacctccgcctcccgggttcaagcaattcttctggctcagcctcccaagtagctgggattacaggcatgcgccactacacctggctaattttgtattcttagtagagatggggtttctccatgttggtcaggctggtttagaactcccgacctcaggtgatccacctgtcttggcctcccaaagtactgggattacaggtatgagccactgtacctggcctaaattttttaatgggaaaggctatgttaaagagaaatccttttgtcccatggtctctcttttctgtaaacttttcgtttgtttgtttgtttgtttgtttgtcttgaaagacagggtcttggtatagccattgcacccagcctgctataaactttcagttttcttttctccagaaaagacaatcatgtatgtgtgtatgtgtatctgttaccttctttcctgttgtattccagttcaacttttttttacttaatatttttaaaagtaattatttagccgggcacagtggctcacacctgtaatcccagcactttgggaggccgaggcaggcagatcatgaggtcaggagttcgagactagcctgaccaacatggcgaaaccccgtctctactaaaaataccaaaattagccggacatggtggcaagcgcctgtaatcccagctactcaggaggctaaggcagtagaattgtttgaacccaggaggcgaaggttgcagtgagccgagattgcaccattgcactccagcctgggcgacagagggagactgtctcaaaaatatatatttatccaattggctttgatttgttgtcttagatcttaaaatgttgtcagacctttgttctgatgctgcaaaatcatctgcatattatatagatgaaatcaacaaagagttatgttcttttattctatcacatttaatatcggtatgcctttggataaaggagaaaaaatccatgtacccatagagaaagacaggagggaaagatggggaaagatgaagagaaatttaaaaccagtctttccttaatctagatctccttacttatcccaatggtagcatttatggaaaatttgctgcctgtcagttactttaatacattgtcataaatgctcaatagaaacataaagaaggtgttattttcatttcatgtatgaggaaatgaggcaaagcaaggctgtaaaccctaattgatttatccgaatataaatcttctctgttggtttgcctgctgctactctctctctggctggtcatatgtttccctggggaaagatcattagacaaaacgttaatgattgccttggctacagaaaaaaccaggttagcctttgccactttttatcatttgtggattctgatcgctcaaaagcttagatttttattattcaaatctaaattccagtctgttcacattctgagactaatgttcacatttataaattttatgctccctggactgataatatgtatatatgcttcgattcttatttaaaaagttagttttttctgcatataaattaagtaaagcatattctttttcatttatttactcttaaatgcaagtgactttttctttttttttttttagacagagtctcactctgttgcccaggctggagtgcaatggcatgatctcagctcactgcaaactctgcctcccaggttcaagctattctcctgcctcagcctcctaagtagctgggattacaggcatgcgccaccacacctggctagttttcgtattttcagtagagacagggtttcaccatgttggccaggtggtcttgatctcctgatgtcatgatccgcccaccccagcttcccaaagtgctgggattacaggcgtgagccaccacagccagccgcaagtgactttttctaacttcatttcagtgttgcctgtcagcattgatcttggtaatagaattcccattgtgttttattagtttgagttatataacatttttattctctaatttttttagacaaaatatagtcccaatatattaaactttgtcttggcctcgttgctttaaaatttttacaagtcattgccagtgactctcaaactattaattgttgatgtgcacagtgactgatgcctgtaatcccaacactttgggaggcagaggctggaggatcacttgagcccaggaatttgagaccagcctgggcaacataacaagaccacatctctaaaaaaaaattaattaactgtacttgaaataaaattgtatattcctctattttagtagctttctagtcttttgaattagttttttattttaacaaatctgtttaggaggctatggaaagaattttaaaatggcctataatgtcatttgatattttgcactgtaggtgtaaaaattggaatttttttctgctacttgaatttataattaaacatccaggttctatttatcacatagcctcacctacctggaacaccagcagaaaagggaagttttgtttttttttttgagacagagtctcgctgtgcgcccagactggaatccagtggcaccatcttggctcactgcaacgtccccctcctgagttcaagcgaatctcctgcctcagcctcccgagtagatgggattacaagcacacgccaccacacccagctgatttttgtatttttagtagagatgaggtttcaccattttggccaggctagtctcgaactcctgacctcaggtgatccacctgcctcatcctcccagagtgctgagattacaggcatgagctactgtgcccggccaagggaagataattttttttttttttttttttttttttttttttttgagacagagtttcactcttgttgcccaggctggagtgcaatggcgcaatctcggctcaccacaaccttcgcctccagggttcaagcgattctcctgcctcagcctcccgagtagctgggattacaggcatgcaccaccacgcccggctaattttgtatttttagtagagacagggtttcaccatgttggtcaggctggtctcaaactcctgacctcaggtgatcagcccaccttggcctcccaaagtgctgggattacaggtgtgagccatcatgcctggctgggaagataattttttttcttttttcttttttctttttttttttttttttgagacagagtctcgctctgtcgcccaggctggagtgcagtggtgcaatctcggctcactgcacactgcaagctccgtctcctgggttcacgccattctcctgccttagcctcctgagtagctgggactacaggcgcccgccaccaggcccggctaattttttgtatttttagtagagacggggtttcaccgtgctagccaggatggtgtcgatctcctgacctcatgatccgcctgcctcggcctctcgaagtgctgggattacaggcatgagccactgcgcctggcgggaagataattttttaaaggcttctgagcaagcaaaaaattgatgtcagaaagttcacattctaaaacttattctaaaattttacttaccagagaatctcttcagttttcagagcctatttacctatattttaaaacaggtttaagaaggttggctaagattttctgctactcagcaagctagtagcagcaaggagcaatgtacatttgtagcagtaaaaagtaacttataacagggaggggatagaaaaatcataaaaagccgaagtgagacttcagaacttaggagttcagtcaaacttaatctgtgtagcttgaacaaatggttatatgatgatggggtatgtgtagctatatatccagttaggatacctagtcattaatattaaattatagtactatagtataacctgttgaagaaaatagctatgtataaaagcagtgtgattttaagtgtttgcttttttttttttactttttaattattttagagacaggatcttgctgtgttgcccaggctggattcaaactcctgggctcaagcattcctcccacctcagcctcctaaagtagctgggactacaggagctggctgtttacttttttgtttgtttgtttttgagacggagtcttgctctgtcgcccaggctggagtgcagtggcgcagtctcagcttactgcaacctccacctcccgggttcaagcaattctcctgcctcagcctcccgagtaggtgggattacaggagcctgccaccatgcctggttaatttttgtatttttttttaatagagacagggtttcaccacgttggccaagttggtctcaaactgctgacctcatgatccacctgccttggcctcccaaagtgctgggattacaggtgtgagccacctcacccagcctacttttttaaatctaaaatttacttgtggaatagaccattcattctccatggcctgataacttagatgtataaaacttggtttaagatttttttttttacataattttaaggtaatgggtgaataaagcttaaaattggcttttgtgtttcttctcccacagacagctggaaatgatccctattgttttgtggagtttcatgagcatcgtcatgcagctgcagcattagctgctatgaatggacggaagataatgggtaaggtaagctgtcgtaattaaagaaggtgacttgcactgaaaaaatttaagttatatatgattttgtttcctttttagaaacattaaacactttttttttttttttgagatgaaattttgcccttgttgcccaatctggagtgcaatggcacagtcttggctcactgcaacctctgcctcctgggttcaagtgattctcctgcctcagcctccctagtagctgggattacaggaacgcaccaccacacccagctaatttttcgtatttttagtagaaatgaggtttcaccatgttagccagactggtctcaaactcctgacctcaggtgatctgcctgcctcggcttcgcaaagtgttgggattatagatgtgagccaccgcgcctggctgaaacattaaacactttatagcaaaaggtcagttattgacatataatattaaaaaggtaggataaagcttttagagggcagaggcttgtgtttatgttagtattttcttttctttttttttttttttgagttgggtctttctctgttgcccaggctggagtgcagcggcgcgatctcagctcactgcaagctctgccttccgggttcatgccattctcctgcctcagcctctggagtagctgggactacaggcgcccgccaccacgcctgactaattttttgtatttttagtagaaacagggtttcaccgtgttggccaggatggtctcagtcgcctgacctcatgatccgatctgcccgtctcggcctcccaaagtgctgcgattacaggcgtgagccaccgtccccaggtttttttgttttttttttttgagatggaatcttgctttgtcacccaggctggagtgcagtggcaccatcttggctcactgcaacctctgcctcatgggttcaagcaattcttctgcttcagcctcctgggtagctgggatgacaggcacccaccatcatgcccagctaatttttgtatttttgtagagacagggtttcactctgatggccaggctggtcttgaactcctgacctcaggtgatccgcctgtcttggcctccgaaagtgctgagattacagacgtcagccagcagcctatgttagtattttccatggtggattgtacatggtaaataacaaaatatttgctgaattactcaagcttggatgctaatggaaaagaatgaaaggaaaaaaaataatatgggtactttaggtaggaaaggttaattgaatgataacagacacataaagattaggatttgtcaacattatttgcctaaatgtaggtcagaagattaagtgttgaaacttgaccagagctggtatctccttcagtgtttcatcttaaacagatatcttcctatgtgtttatagatacaaatgtgattttgggctttatttttgtgaagtataaatgtagaaattttttttttttaggaagtcaaagtgaattgggcaacaacccctagcagtcaaaagaaagatacaagcagtaagtatattttatgctctttgaacatttgtttttattgtacccagtagttttattgtaaagcctataaacatcatacatgtttgcagtaatgttttgatcgttatcctaagatatgattgaatgtgtttgtgttaataaatttaagaacaaatctaatctttgtcatcaggtagtaccgttgtcagcacacagcgttcacaaggtaattgtatcttcttaaacataaaatgaaatctcttgaaagggtattcactaaccacctgaagtttttttgtttgatatttgggggaggggggcgggaggagatattatttctatttgtctctctagcaatacttttctcccttctcagtgttgaccaagtataacttgtccgctactttggtgctagttcaagctttctgacccgtttggtagcagctgatgccttagaactactttcaccaactaaggggcaaaataccctcctttcttgaggtcaccatttgggcttcatacccagatcttccaaatgcttgagttgctcctcaaattttgtttcccaaagagcaatccaaaatgttgtttaaggcctgtcaaatatgggtaacttttctttccaaatatgctttgtcaaattgatgtatgtgtccattttaaagtgttggtccaacaattttgcatttttaaagtgtttctttttttgataattgtcttttttaaaaacttcagatatgggttggttatttctctccaatgcttttttaatggttctgatataaagtgaagggattactgttttcattctgttgccttcagtcttagttcacttgcacatggattcacataaactgaatggtgtaatgtctgggcaaccaaaactgttggcttttgagaaaactgtcaaatactttaacatcaaactgttgcaatgcaaggtatttctttgattgttcttcacaaaatatggttaaaccaagtatatatcatgtagctagcttcagtaaattgtgttaactgaggcaaatctagtctacataattcacagtaccactattttattttaatttgtaaagccttaatatagtggtaaactgaataaaagtaaataattattattagaatggtaactaagtcattaaatttttttgcagaactgaaacttgtatgttattagtttattttcttagaccagtgtaataattgactgtaaatagaaatataaatgtcactttacagttagatgtatcacagtcgtttcaggagaatttttcctatattgttaccttgattcattgtttaaaattggtaggatttgtatagatataggatagtgttttatttatactttatcataagccataatcattttaagaatactttattggatagattttagtactttttaaattctaaagttctatttttcttttcacttccccttccttccccttataagatcatttccatgtctttgttggtgatctcagcccagaaattacaactgaagatataaaagctgcttttgcaccatttggaagaatatcgtaagtaacagaagataaataaaatccttttaattagaaacaattatatgtagacataacttgaaaataattctgttgtgaattttgtacattaattttataacttgaatataatgtattacattgtttagagatcattcaagaagttaacagcaaaagattgttttcccattgatgaataaacctttggttttcaattatcttaaaatcctttttcaattagcatagctcttaagagacaaatttgccattgtacatgttttactttcttatataggtatcaagctagcttggtgacaatctttagtaaaattgtgctgtaactaaggaatttagatctgttgggtacttctatgataccatgcattttgtgttaatatattttaacaaaaagaaaccttaacaattcaacttaaaaagcagttattgaatattattttctagatatttacacccttttaaaaaaactgctaacaataatagtgccttaataatattttatgaagcactgccttgcttgcacttgttattagtcatacattttaggcccttatgaataaataccttacttgagatagcattcatcatagaagaactttaaaattttgaaaagcatgccatattagagtaagaataacaagttgtttacagtactttctgtagagtaattagcttagaaaacttcttcagggtttctgcttgctctgttatttttccacctagaagaatagtaaaaaggaagtgctagctgtggtttggtttgaacagaccaacagcagagatgagatgaggagactggaaagtaaatgccagtagtattggtgaattttaggagctttcgtttgaagagttatcgttccttgattaggggatgtgaccaaggaattaccaactttaaaggtggtcacagaattagggcttataaatggacttctttcagatgacatttttctttgtgagacctgatcaggaaaataagtacagctttcatttcttgattgtccttcttaataatatcttagagtttcaataataattagcttgctttcgaatatctggacttagacagtcttagcctttggcctctcaagggctctaaatctctatgcacaattttgtgttttcattttggtgtggaaaatctttgtaaattttatcctattgtcagagtagtcatagaaccataaaaggataaagaagcctgatctagatctttgctgctgcttcagagaatcttttcagagagaaataaatgagttgaatatttttcaagactaacattttctgaaactttaagaagaaaaaattttctaacattgtgaacagattaagtagctattttattaaatgctttgattttaattgaggtagtttttttgaaaacacgggaattttagtttggaattaggcaaagattgtttaacagttggtgagtttaagttaagtgaccttcagattttagcaatttcatgatttacaaatcgtgggttatatttttttctcttaaatatatttctgtgtcatatttttagtctgtagattttcttatggtcccatgattattggtgtatactttttttttcttggtaagacctttaagttttatgattttcaattgagataatgtataatgtttgagttttaggctatataaggaattagctttcctgcatttaaaggaatcatttggcagtcagtagcatattttcctgattttttgttgttgttaaaggagataagttgataactgtatgttgaagggttctcatgatattctctaaaaagtgcaaatatcagtgcttgatttatagatgtgtaataaacagtggtctgtaaaagcatgtagtataaaaaatatggtattttcccttatatacagcctataaactcttcctattccttgagtttagggtagactaacaaattacagcgtagaagagagtcagctaccagattaaatgcatgcgtaccatacagataatttattaagtatttttgcatattgaacattgacttacattaattttcccttttcaccaccacgtctttgtcattttgaccctgaacacttaataaatcactcagattgatctaaaatatgcttatgtatactttaatagaaaattacattgcatggctttctgacttgggtttttgttataaaagtgcctgttttgttcatgtgtcctgagagagcaagcattgtgacatacctgactaacttaaaagcagattgcctgtgaagcacaatttgagtccaattttttgaggtatggagttttagctatcatggctgggtttttactcaatctcaaataatagggctctggttattttgcagagtgtctctgaagaatggacagaattgccctggctaactacaagctacggttcacagtggataaatgttggcgtgcttttttactttctgactttttaaaattttgcttttatatcttgtagttccaatcaattttatatgaatgctattataaaattcagtgtaaaatcttttcttgtctagcttaaaacttgtgtgtgtctagtatttttcttcaatgatttaacatttctgaaaatgtcaagttctcaaaatttagacaaaaaggggggtataaattgatctgaaaaaattaaatagtttaattagtttagggaatatacaactttttcattcttttggtttccagttttctatttttttttaatgattaggtagaatatagtgtcaaataagttataaattgcatggttaaagaaagccattcatctaatgtcctgtgaactttataagtggttaagtagttcatgtgagtagctgctttcaccatgcttaatatttaaaagtttaaaaagttttttgaagagagaggcaatttctgtacaatataaatatacatatatacttaagttattgatttgcatgtctataaaattaagcctcatttcctaatacatgataaattttgtagaataaaaggtgaatttgcattaaaggttcactttaatgaaggtgaaacatgagaatgagttgtgtgtgaaattcttatttgatatccagaggatagtttgcaaacctaaacttctgattgtttctgatttcagagatgcccgagtggtaaaagacatggcaacaggaaagtctaagggatatggctttgtctcctttttcaacaaatgggtgagctcagtgagaagtgcatggataatgcttaaagagtaaagaatggaaacactccgttttaattactaatctttttttcgtctgctgtttattttactgatgattaaaataaaccctcactagaagatttcttctgttttgtctgtttaacatttattggcacctgaggcatgcagagtactgataagatgttttcagaatgtagaaagtaaagttgtttaaattatcagccaaaggtagatgttgaaaatctcatttatggatttaattcattatctccctagtttatattcccatggtagtttaaaagtagctaataaaatactgtgaggaggccggacgcagtggctcacacctgtaatcccaacactttgggaggctgaggcgggtggatcacctgaggtcaggactttgagaacagccaggtcaacctggtgaaaccctgtttctactaaaaatacaaaaattatccgggcgtggtggcaggcgcctgtagtcccagctactcgggaagctgaggcaggaggatcacttgaacccaggaggcacaggttgcagtgagccgagatcatgccacagcacagcctgggcgacagagtgagaccctgtctcaaaaaataaataaataaataaaaatactgtgatggataggaactttttaaaaaattaaatattacccgggcacagtggtttatgcctataacctcagcactttgggaggctgagaagtttgagataaatactggcaatatagtgggaccctatctctacaaaatatatatattttttattttactttttttttaatttttttttttccgggaattccgaaaattcagccgagcccagtggctcacacctgtaatcccagcactttgggatgccaaggtgggcggatcacttgaggtcaggagttcaagaccaacctggacaacatggcaaaaccccatctctacaaaaaattagccaggcatggcgacacatgcctgtagtcccagctacttgcagggctgaggcgggaggatcacttaaatccaggaggccaaagctgcagtgagtcatgatcaagccactgtactccagcctgggtgacaaagtgaggccctgtctcaagaaaaaaaaagttgaaaattgtaaagtacattgagaggtgaaaacaatacaagctcaatcatgtacatgtcccatctaccaaagattttttttttcctgtaagatgagaaaataaaaattaagagaatttatttggttctcaaaactttactattaagcatagcataatgttttagaaaggtagaaaaataccttctataatggattgatagccgtgattaatttgagttccttagaggattgattcataggttttaggatgttttagtgcaagttagggaaataaattggcggtttgatgggctaggaactcatttttcacattttcgagattgtggactgtagggttctgctatataaaaatcactgttgaatgtgtatttagtatgaactagagtcaaatagcaaaggatgtctcttgataacctgtgtatggcctctggagttagccagagatgtatacaaattctgacaaccattataaaacagatgtaactttgagtaaataatataattttttttttgagacggagtttcgctctgttgcccaggctggagcgcaatggcgcaatttcggctcactgcaacctccatctcctgggttcaagcaattcttctgcctcagcctcccaagtagctgggattacaggtgccgccaccatgcacggctaattttttgtatttttagtagagacaggatttcactatgttggccaggctggtctcaaactcctgaccttgtgatccacctgcctcggcctcccagagtgctgggattacaggcgtgagccactgcgcccggcagtaaataatataatcttctataaaatcttctataaatttaaatctataaatttcaacttatgtaaaatttgtgatacgtgcttggctctgttacgcagtgctacagccctgattgcaattacccccagcctctcttcctatgttctacatgctaggaagaccacaatactgattgagcttagcttctctgcctaccccaaaataactgaatgttcctggaaaaaaactgagttcaggtggttttgcttctaatttaatggtcacacactttaggtgagtacagagtactgtcttggcaaacatgtgtttctcatctttcaaccaccaaacttacataaaattcatccaccttcccattcttcctattatagtacaggaaaggtccttcctgtcaaaggcaaaatcacttatgattgtgtccccatctctcttgccttttcaaggactttgagcctatgctgccactgcttcattgttttttttttgtttgtttgttttgttttgtttttgttttaacagcagctttattgagatataattcaagtatacagtgtggccgggcacagtggctcacacctgtaatcccagcactttgggagactaaggcaggcagattacttgagcctaggaatttgagagcagcctgggcaaaatggcaaaaccccttctctacaaaaaatgagccaggcatggtggcacacacctgtagtcccagttacttgagaggcagaggtgggggaatcacctgagcccaggaagttgaggatccagtgagctgagattgcaccactgcactccagcctggacgacagagaccctgtctcaaaaaaaaaaattttttttttaaagtatacaattcagtggtttctaggtaattcacagaattgtgcaactattaccaaaataaattttagaatatttttaccaccccaaaaagaagctacatgactcattagcagtcactatcctcctttcatgcccctcctgccagccctgaacaattgtcaatctactttctgtctctgtagatttccctgttctcaacatttcctataaatggaatcataatgtgctgttcttgttattactgctattctgtgtcatcactctattccttctctactggatcattctcatcaacctaggatctctcttttttttttggagagagggtctcactctgtcactcaggctggagtgcagtggtgtgatcatagctcactgcagcctcgaactcctggcctcaatccatcctcctgcctcagcctccagagtagctgggaccacaggtgtacaccaccatgcccacctaatttttttttaaaaaaaggccaggcatggtggctcacacttgtaatcccagcactttgggtggccgaggcaagcagatcatgaggtcagaagttcgagaccagcctgaccaacatggtgaaaccccgtctctactaaaaatagaaaaattagccaggcatggtggcacacgcctgtaatcacggctactcaggaggctgaggcaggagaatcgcttgaacctgggaggcaaaggttgcagtgagctgagattatgccactgcactccagcctgggcaacagagtgagactccgtctcaaaaatatatatatatatttttttgtaggaacaatctcactttgttgcccagtctggcctcgaactcctggcttccaccagtccgcccgccttggcctcccaaagtggtggggttataggcatgaggcaccgttcctggccatctagtatctcttaatcaaaaccactgtttatcctcacatcccttccctaactgtatccccatttcttggctataattctcaagagttgtttgcatatgcttctactttatctcccattcacaaccaccaacatagatacatctctgttttttttccacttttttgagtgagtagttttatttttttcctgaggagctgtatcaaagtatcataaactggatggcttagagcaacaaaaatttattttctcaatgttttggaggcgtgaaattcacaatcaaggtgtcagcatgtccatgctcttttgaaggctctgtagaagaatcctttgttgcttcttccaagcttctggtggttgctggtaattcttggcattcattggcttgtagctgtataactccaatctctgccttcatcttcacatgaccatcttctctctttgtgtatccatataatcatcatctttttttttcgagacaggacctcactctgtcacccaggctggagtgtggtggcacaatcacaactcactgcagccttgacctcccaggcttaggtgatctttccatcacaacctcctaagtagctgggactacaggtgttgcaacaccatgcccagctaatttttctattttttgtagagacaggattttgtcatgttgtccaggctggtctcgaactcctgggctcatgatccacctgcctcggcctcccaagtgctgggattataagtgtgagccaccgtgcccagtctgatctttacttcttatgaggacttgagtcattggatttggggcccaccttaatccagtataacctcatcttaattacttcggcaaagaccctgtttccaaataaggccatatttcacagataccagggtataggacttcatatcgttttgagggacacaattcaacacataacattagttaagcgtctcatggagttcggactggttggggtgataatatttgtttctttttaagaatgtgcatttcctgccaggcacagtggctcattcctgtaataacagaactttgggaggccaaggcaggaggaggactgcttgagcccaggagttagaaaccagcctgggcaacagcaagaccctgtctccattaaaaaaaaaaaaaaaaaaaagcctggcgacctagtctgtggaccccgaatccctcccattgcctgttgcaaccagtaaaggctgttcctaccatagtaaagaaattaaaaaaataaaaaaaacagccaaccttgttggtgcatgcttatagtcctagctactaggaggctgaggtgggagtatcacttgagcccaaggagttcaaggctacagtaagctgtgatcatgcctggatgacagaataagaccctatttaatttttttttaaagcatttcgttttcatatttcaagaaaggccttgaatttgcctgttccttcagtagtagattaagcttacctttgatgtgtgcacacaaaagttacactccatttactttttagtctactctaagctggttttactctcactttgtaccaatgctactctattttcaaggtcacctcttaacctctgttttcttggctccctggaccatacacttttgtggctttcttccttcatcactggctactcctccctagtcaccttagctagtgattattcctttacccatttttcaagttttgctgtttttcagggctctgtcctaggctctttctctagtctgcatgaattttgtatcttcagtctacttctctgcgttccacatttattaacaaatggtcttcatttatatgtctcacaggcatctcaaagttttttcttaatggagctttttgtttcttcctcagacctgttccccttttctttcctagtctttctccgcatttcaataaatgctgtctttccaatttctctagaggaacctaagagttatcttttctctccttttgcttgtggtgtcccctccagcccccattattttggccttgagtttaagttacattcactgctactgcaatgctcctggccaccaacactttttaaaaatctagaatattgcacaagtttcataaagtatctttatccccttactacccattatcctaagccattctcctcttagcgctaagaatggccttcttgccgggtgcggtggctcacgcctgtaatcccagcactttgggaagccaaggcaggcggatcacgaggtcaggagatggagaccatcctgtctaacacggtgaaaccctgtctctactaaaaaagaaaccctgtctctactaaaaaatacaaaaaaaaattagccgggcttggtggctggcacctgtacaggagaatggcaggaacccaggaggcggagcttgcagtgagccaagaacgcgccactgcactccagcctgggagacagagcaagactccatctcgaaaaaaagaatggccttcttaaaacataggtcatgatgattccttgcttaaaatctttcagtttcattctctgacattcttttttgctcgctggattccactgaaactagtctttcacatgctgctttcagatgcataccaaacttttgtgccttaacacactttgcaatacttttctctctactggggctttcttcctcattgttttgacttggctatctcctattgctgtcaggcttagtttaaatgtcatttcagagagacctttcttattcatcctgtctgtagtagatccttgtgcccatattctctatctcagccttttgtttctgtcataggtcttaccacaatatgtaattgctttatttgttccccacaaaagaatgtaagagaccatgtatgcttgtttaccagtgtgtccctagctcttaaccgcatgcctggaacataactgttcaatatttatttgttggatgaatgagaaaccaaatattgttaacaagcttgttagattgtttaagattttcttctatagtagtctgagttgcatgaactttatgatatctacctcattagggctgtcgggctcacgaccttttcagatgattaatgttcccaaaatttaatattcaaataagagaaaaacaatacaaaagcaccattgggttgagcatggtggctcatgcctgtaatcctagcactttgggaagctgaggtggacagatcacttgaggtcagaagtttgagaccagggccgggcacagtggctcacgcctgtaatcccagcactttgagaggccgagttgggaggatcacgaggtcaggagatcgagaccatcctggctaacatggcgaaaccgtgtctctactaaaaatacaaaaaatgagccaggcgtggtggcaggtgcccgtagtcccagctacttgggaggctgaggcaggagaatggcgtgaacctgggaggcagagcttgcagtgagccgagatcacgccattacactccagcctgggccacagagcgagacttaaaaaagaaaaaaaaaagaagaagaagttcgagaccagcctggccaatatggcgaaaccccatctctactacaaatacaaaaattagccaggcatggtggcagatgcctgtaatccccgctactcaggaggctgaggcaggagaatcgcttgaacccagttggcggaggttgcagtgacccaagatcacaccactgcactccatcctgggtgacagagcgagactctatctgaaaaaataaataaaataaaatttaaaaatataaaaagcaccattgcattttaggaaagctatcatatgttgtgtatttcatagagcattttcacttaaacatttgtgaaaagcaccaagttttctctactgctaagagttgtttaacatcaactaatagtgacttctttattgtttctaatgttgtacggaacaggatgctgaaaacgccattcaacagatgggtggccagtggcttggtggaagacaaatcagaactaactgggcaacccgaaagcctcccgctccaaagagtacatatgagtgtaggtgtattggagaagaaaaggaaatgtggaattttggagaaaaatacgctagattttaaatgttagagctgttcccggagacttattgcagaaatagatgagaagcaaatcaagactactattcaaaaatgtacttagttttcatttttgtaattataaataatattatttctaatgtcaagtctcctattaaatagaaaatactgggtaattttttagacattcgtgggggaggtttaattctgaactgaagtataatatgtgtaatataagcattttgatcaaggttaatgttacctttacagtagtgtagtgaatgctacttttctctttctgcacattttatttaatgaattaaagacataaaccttttctctcaccagcaaataccaaacagctatcatatgatgaggttgtaaatcagtctagtccaagcaactgtactgtatactgtggaggtgttacttctgggctaacaggtatgggagccttccctgtgtggcattaatttttaaagtgcaaagcttaatatttgtaggatttttatataacttagtgtgatttgtttgttttggttttttgtctaacagaacaactaatgcgtcagactttttcaccatttggacaaataatggaaattcgagtctttccagataaaggatattcatttgttcggtagggatggttttttaaaaaaacatttttcttggtaatgtaactggaaaaccagaaaataaataaaaataaaaaactttttcctccaaaatattttttgaacatttacctcgttttcttaaaaatttaccttggttataaaaaagttgaaattttggccgggtgcggtggctcacgcctgtattcccagcactttgggaggccgagacgggtggatcaccgtcatgagtttgagaccagcctggccaacattgtgaaaccccgtctctactaaaaatacaaaaatataaaaattagccggcatggcggtgcatgcctgtaatcacagctactcgggaggctgaggcaggagaatcacttgaacccaggaggtggaggttgcagtgagccaagatcacaatcactgcactccagcatgggcaacagagccagacaccatctcaaaaaaaaaaaattgaaatttctctagcatccagcgtatttgcctctgtgattttatagctccctccctaacacaaggccagtgaaacacacattctttgttaacacaagttgccccagaactacaagatctttgaaaaaaatagcaaagaaaatatgccatgttacgcaaactgttttacctcagactcaccttgatttttaaaaataggttttattcataaatatttaaaagaatttgtattggtgattcttaatattgttttcaataggttcaattcccatgaaagtgcagcacatgcaattgtttctgttaatggtactaccattgaaggtcatgttgtgaaatgctattggggcaaagaaactcttgatatgataaatcccgtgcaacaggtgagagggttcttaactttgagaagtaattgttgggctaataatgttattttaaggtgaagattgttgataatccttgttaataagtttctagctaaaatgatagtatataagggctcttcctacatgtattatagttccagaatggtgtttcatggattgcagtggtgacatgatgaatgtgaccgcctcaattgactcatgctcacacagtggattttaaaatgctccgagaaacctaaaagggtattacatctttttttaatacttggtagatttattcacataccatttggagaactcacaagcatgcttgactcaactgaaacaaaaataaagctgtaaatatagttatggccttggcatttttcttccagattttgctgccttttttttccaggcagctttagtggttcaaacttgattccacaaagatttctatgaggaaactgtaaatggagcacagagatgggagtaagcaaatcctaaactatataagtgtcttaagtaagctgaatatagaaataattaatcacatcttccctatctaaaggaaacaatcatgtagatagtataccaaacacttaaacattctactaagtttaacatttattactagggtatcaactgatgccttccaaaaactatgccattcagagtatcacgtagagttacagcagtataacctcacttgttttgttgttgttttttgttttttttttttttttttttttttttagcagtcaactcttgtttatcctgtcattttaattgtaatgatggccctgtgtgttttggctaagaatttgtggtatcagcatattaggattgtatatatatagttaacttcaagtaatactaaattttatttaatagcagaatcaaattggatatccccaaccttatggccagtggggccagtggtatggaaatgcacaacaaattggccagtatatgcctaatggttggcaagttcctgcatatggaatgtatggccaggcatggaaccagcaaggatttaagtaagcatatcttatgtcttcctttactaacctttgaaaatgtctaaagaaattttaagtaggaagaaattttaagaattagtgggtaatagggcctcttatgtaaagcgtaactatagccttgaaagaacttaaaattgttttttagtaagaaaataagagcttaatagttgttaagcttttcacatgactaatggtcacttagccattcttatatacactgagtttgatgctagttgttgtattcattatgcttttttctgtacaaagggttttttcaatatgccttgttacattgcaagcttccttatacaccctgaattacactattttacttacatttctgaaaaacacactggaaataaaaatacttgagaggttaaaaaggaatttagtgggctgggcgtggtagttcctgcctgtaatcccagcattttgggaggctgaagtgggtggatcacttgaggccaggagtttgcgaccagcctggccaacatggcgaaaccccacctctgctaaaaatacaaaaattagccaggtgtagtggtgcacaccagtagttccagctatttgggaggccgaggcaggagaatcacttgaacctgggaggcggaggttgcagtgagctgagactacaccactgcactccagcctgggcagcagagcaagactctgtctcaaaaaaaaaaaaggattttgtggttgggcatggtggctcatgcctataatcctagcactttggaaggccaaggcaggcggatcacttgagtccaggagtttaagatcagcctgggcaacatggcgaaatctcatctctacaaaaggtacaaaaaaaattagccaggcatggtggttgcgtgcctataatcccagatgttcgggagactgaggtaggagaatcgcttgagcctggggagacgtaggttgcagtgagctgagatcataccactgtactccagcctgggtgacacaatgaaaacctgtcttaaaaaaaaaaaaaaaagaaaagaaaagaaaatcttgtaatgttatgaatttttatgagctttcatatgctaataattgttttgatttcttctttgagtagtataatgttcctttttcgttgtagggaacctacctgactctttcaggtagtggtttcctcagcaaatatgttcaaccaaggcatagagatgagatggatagctttcttttccaagaatggggttaggtttttctatattcaaaggagatgaatgttgataagttcagggtattttcttagctttaatttctaacaaaattaaactgtccaaattaaacatacagggatgcttgtttttaagagaaaaaaactttagtaaaagatttcattttccctagcataaattattctatttgggattaagaaaaagtatgtttcccatttcccatatttgttcttgttgtttgttcttgccaatactttattttaatcagtggatttcctctcccctctgcttctttcacattctccaccagtcagacacagtcttctgcaccatggatgggaccaaattatggagtgcaaccgcctcaagggcaaaatggcagcatgttgcccaatcagccttctgggtatcgagtggcagggtatgaaacccagtgaataaggactccagaatctaaagccagtggcttgaggctacagggagtgtagtaaagccgttgtttacttaaagatttatcaaatcagtcagtgcaaatgtcagatacaatgtatttatttaaaagattcatttttaatcatgaaattacttatcatccacattgttttaaaaagaaacaagatgctggatgtctgccaatttttgccttcattaccttttttgataaagtttctcagatccttgtttcaaacacaaatgcagggattgctgccactttttaactattaagaggcagaaaattgcacaatattgaacttttttccactgaagtagtgtgcagttctagtttgcattcctgatatgatttaaaacatgtaatataaagatgttaaaaaaaaaaaccaaaactgtgcagagtctagaagttgtttgtcatcttcagcttgtgcacaattctgttttaggttaaaaaaaggcattgtttgagctgtcccatctccactgttatccctttggggttttttaatataaattattagtttacatcatttttgtatctacatcttttttcacaaatttgtcttgccttattaaagttctgtaaaatatacttaaatggaaaaaatgatgttcatttagattgaaaacttttctcagatggattgataattgcattcatcttgtgttttatatgagaaggtgcctcaagaatttcctgttggatttgtttaaaaggatttttatctttcgtgataaactttgctgtgtaccaggaactataaaaacaaaaacttgttactaaagaaaatatctgaaatgtgataagttcttatgccatgttaatttcatgtgtcaacttcaacatttacatgtattatttcattatgtaaaatgttttagcaatttaatattttgcacagttagcaaactttgtatgtcatttccttcaaggcatcatgcagagttgacatgagatttataaggttttaagttgtttgcatgtgaaaatcaaatacatactttggtagtctttgaatacaaagtcatctgctcttgtttttcaagaattttgagacacaaagttgtatgtaaaggaatatattaatttgccgttttctaggtagatttgctcaaaaagagtgaatcaacttaatatgtacaaatgatagctgtgaaactgtagaatatctttgtgtcaggcttggagttcattgtgacctccaaattttgcctgaaggaccagctgggcaaagcatcttttaaatgttcagaggccaaaagataaacaaaaaaaaaaccttaaaatcctacctctttaaacagccttcagataagagaatcctcagtgcaatcattattttgattcgtttggtacctgttttcctggagttcccgattttattattttggggtggctccaagcattaagaggtttaatctttgatggcattgttctagttttgaaatttctagtatatttcagagtctcttagaagacttgtgtgggaagtttcactttgttttcagtgaagatcacaaacctccttcttcctttactcaagaggaaaggtcccagtatacatatttgaatggttgatggttttcaagaccttcagggagctccctgcattttacctagaaacagaaaaggcccgcaaaatcttaagtttcctggcctgcatttcccgggtaggggcaaatgactccaagctggtctctaagccaatacccttataaaccagagcccaggaaagacagctcgagtgtataattctctggagctcaattctatgcagttgtgctgatatttcattaagtcactgtgtatttttaagtgttgatacattaaaagtcgctttatggaagatgagtaaattttttaaatacttggaaattttatttccttgttaacttctacagatcagggcatgcaaccaaaagcagcttaaatgaaatattttaaaataaaatatcaggaagctatttttagatttcttctggcttatgtttctactttaggaccctcattgttctcttattaaaaaaaattatttcctgtgcatctcatggactgcagggtaaattatttgggcataaataatttaaatagttttctttcattttgactatctccagtaataacagtttttattatccagcatattggcttattgcacaaatcttaaaatgtacattgactactttctgagaagaaagtggtatcagtactcatgatgaaaaggttactactgaacaaattcacatttcaggaacacctctatctttggtttaaatcttactcttagtttttccgtctaaaaatcatactggtattagtatcaggtaaggaaattaaagtttttaaaatggtttcattctctgcaatatgcaaaatttagattttactttctggtactgtaaagaacctgaagtgatttacacttaatgggtgattaatccagtattctttaccctgaatgtttggatattaaagttcctttatgttttctataacctgtgggatcttcttgcagtgattattgtgtgtgagattttttttctttttggtctatccatattgttatattcactcaggtattttttttttaatcttattccagaatcagtggtttatattgggttactgtttaacaccaaatggaattggcattctgcagatttaattaattatgaaaccagggtctcattttccttgctgatacttgttgaaaatgagattcacattctagtctttattttcctcctgttttgtccctgtgcttgtacatcttccttttatttgtgtgttatagttctattccatttgagaaggcagttggtaagaactagattgcatgtacaaagacaggtttactaagtgctgtacagtggtcctgaggttacagttgaattagaaaaacgaaatgtacttacaggaaataagaaagcaaacctttcaaatgagagtgatgatttctttaaaaaaaatcagtttttttctctcaaataatgttctttatttcacgaaatcgtcaatcttaagcatgagcagggataaacaactcctagaaggaactcaattcattcttcctggattttctctgttgttaaatcacaaaaatgatagtccccaatcgtttctttataggaggttattacatttcattacagtcactgcattttgactgttgtgtttagaatttgaatgtacatccaaaatgatgagtttcaatttaagagccttaataaaatgtgtgagtgtgtctcaattgaa

SEQ ID NO: 2 - DNA, 37988 bp, Macaca Fascicularis
  misc_feature (6467)..(6566): n is a, c, g, or t
  misc_feature (7243)..(7342): n is a, c, g, or t
  misc_feature (28791)..(28868): n is a, c, g, or t
attctgcgcctgcgcggcccgggtaacttccctggctgctcctaggcgcccggctagccgccatcttgtattggggcttcattgttcccgctgggccgggcggtttagtgtaattgccgcgggagaaggaggcggagtaacctctggtcagccgagaaaccccgctatcccgtagccataaccgctcaaacgatttgggaggtagtgaagggcagggagttggacccggaggcggcgccgcgacagcagcagccatggaggacgagatgcccaagactctgtgagtctggggcagcgatgagggaggcgggatggtggtcgtcccggagggaaggcctcggccctgcgccgctcccagcctattgttctccgtggacaccgccatatcgtggtttttgtttccttagcatgcacctgaatgcttagaaccaagacccaatctgtgagaggacaaggtgaagggagaccgggctcaaccctttcaattaatcccttcactttagctacaccattgcttgacctttgctctcgccgcgggccctctgtggtcttagacctggagaagactttttcattcagatctctttttagaacttgtcgaaccttccttttgtcctatgctttctttccttctcttttcccgcttctccccctttttaattaacaacttcttatctttacagccgtcttcctttaattttacttttgatttcctattgaaattcttcctttcattttcttcttggtgataagattttttcccccccttgaggctgcttgtactcattttcagtgcggtaaactttatttttgcagttgacattggaaaaaagccaagcatttttctttaacctctggctttccttctgtagccccccaccgaccaaaaagcctaaccagacactccaaggatttggaaatctcacccagaaggaatatatcaattgtggggaaattgatataatattatttttatagtcgcttgctactgttgtgattgacttggtgttgaacgagcgggccgtttttatgaagcaacaattgataaatgccgtaacatatttacagagaatttttagagttagcgttaaattatcggagcgtggatattttctttgtcgtgtggcttcccccactgcgttctttgtttttcaaagtattatcggatgagttgatcctgcctttgttggcatttctcggaaagagaaaagctcaatgagttcttgacatagctttttgtttttcttggatgacatagctttttgtttatattcttggaatactggcagtttgagagctccatgcattaaaaatgcaattttaggtttctctttgcagctttctagataatgaagttgctctgaagtacgatgtcgttgtttttttcacctccaaaatttttccacttttaagttgctatttcttatctatattggaaatgtggttgcacttgttttcttaatagattgtcagttaatctgttagtaaccttgaaatttcttttctctattcctgaggctcaaaaagtatcttttcaataggacgtagcaattagtgcatgacctttgttttaggaaatattgtatctgtagatgaatactccctaaaaaactaactagcctcacggtgttggtggagtctcttcccccttgcctcttagctacgacagccatcagcattttgggttaaggcactgctcgtgttttataatgtaaacttaatattcttcgtttgaaaatgtttaattttaaaaactgccagaagcatctgaccacgtaactttttgtttttactttttaattttttgagatggagtctcactttgttacccaggctgcaaggctagagtgcagtggtccgatctcggctcactgcaacctccacctcctgggttcaagccattctcctgcctcaccctcccaagtagctgacattacaggcgggcaccatgcccggctcatttttgtatttttagtagagacggggtttcaccatgtttgccaagctggtctcgaacttctgacctcaggtgatccgcccagctcggcctcccaaagtgctaggattacagatgtgagccacagcgcctggcctgtcctcaactcttaactattacatttatgacttattttgccttgtatccctcactaaaacaccccatccaagaaacacttcctcaaagtagtcatactgtaaaagtctcttgtttctacctccctcaacccaaactcccatttttccagtcaggcaaaccttggctaaacatgagggtactaatccctaagttttcctagtagtgcttttaattttaatgtaagatttttgctttaatgaccctggaagtcagaggcttttaatatatgttcttaacacagttcttagcacattaaaaacatagagcctcttaatatatgttcttagtctggtccatggtaagtgatcaataaatttgtttaagaaactggtataattggctgggcacagtggctcacgccggtaatcctaacactttgggaggccgaggcgggtggatcacctgagggcaggagttcaagaccagcatggccaacatggagaaatcctgtctctactaaaaatacaaaaattagctgggcatggtggcgcatgcctgtaatccccgcttctcaggaggctgaggaaggagaatcacttgaacccggaaggtggaggttgcagtgagctgaggttgcgccattgcactccagcctggcgacaggagggaggctccatcccaaaaacaaaaaaaaaaaactgggaaaatttcaagcagtgaattaagatttttcttgagaataaaatagagtcttaagtttttatcacccgcatacctaaatggctcactggaaattccttcatttgctacatctgagtgttcagcatctccatgttaagttagagaatactagtatttgatttcttgtcaccaatactttgaaaacatattctgagacaaacttccctaaagtttatactgcaagattattttgaggagtattcaaggtacaattaataagttcaaacatttgtagtgagcgtatgagtgatgtatactactattctctattcatgtataaatttgaaattttccataataaaaaaataaaaatacatgtatagtaagtctgaattggccttggtcactttaaaaatattcaacttgtgaactaaaagctacacactagactagaaaaacattgctggtgttagttgatgaaaattaagtccctcaaatccagacaccttaatatcctttgtgtggtttactctcaaatgcacaagtgctctcttttgagataactatactgttactgtgtggctttcacatttgctaagagtgcagatctataaattatagaccatctttaaaagcctgcagctggccaggcatggtggctcacacctgtaatcccagcactttgggaagccgaggtgagtggatcaccttaggtcagaagtttgagaccagcctggtcaacatggcgaaaccccatctctactaaaaatacaaaaattagctatgtgtggtgacacatgccagtagcctcagcaacttgggactccagaggctcagggaggagaatcgcttgaacccaggaggtggaggttgcagtgagccgagatcacgccactgcactccagcctggacgacagagcgagacagagcccaattaattgctatatactaatgtgagtcttgtctgtcttctgctccatggttatctttcattcttgttgtctaccccactctccaaatagtattggcctttgtaatttgtatgaaataaaatctcttggattgaaatcagacctatcttaaagattagattgaaggcaaggttgactaaaatgagatatttattagataatacttatgtttccaaataactgctagtaattaagaatatgctagaaacattacattcactcagtaatattgagagcatactacatggctgtcactcttctgggaactggggatacggtagtacatgtagtaggccttttaagaatttataggccgggcgcagtggctcaagcctgtaatcccagcactttgggaggccgagacgggcggatcacgagttcaggagatcgagaccatcctggctaacacggtgaaactccgtctctactaaaatacaaaaaaaaattagccgggcgaggtggcgggcgcctgtactcccagctactcgggaggctgaggcaggagaatggcgtgaacctgggaggcggggcttgcagtgaactgagatccggccattgcactccagcctgggcaacagagctagactccgtctcaaaaaaaaaaaaaaaaaaaaagaatttatgtaagtggctgggcgcggtggctcaagcctgtaatcccagcactttgggaggctgagacgggcggatcacgaggtcaggagatcgagaccatcctggctaacacggtgaaaccccgtctctactaaaaaaatacaaaaaaactagccgggcgaggtggcgtggtggcgggcgcctggaggctgaggcaggagaatggcataaacccgggaggcggagcttgcagtgagctgagatccggccactgctctccagcctgggccacagagcgagactccgtctcaaaaaaaaaaaaaaaagaacttacataagtaattatgttagatcgtaataagtgccatggagaaaaataatgcagggtatgcatggtaaggaggaaggggtgaatatttatagagaataataaaggaaggtcatcctttgataagtgacatttgaggaaaaaagtgaaggagataagggagagccatgcttctatctgaaggaggagcaccctagaaagagctagtgcaaggggcccttgggggcagggcatgctggttggttagcctgaggagagtaaatgtagtagaggagtaggaaatgaggtcagaatgataaataggaatgttaggggagatagaaaataccagattatatgggtccttaagaggactgtcttatctgagtaggaatggaagctgtaagtgggtttcaagcatagtagtggtatagcctattttaaccttgaaactggcagaagcaagtagcctagtcaggaagccaatccaggtttgagagaaagcagtgcctataattgtagttagtgctggtgaaagtgatgggaagtagttgaattacggatatgttttgaaaatagagcaacaggaattaccagtggcttagatgtggcatgtgagaatgtcgacaataaatggatttattttaaaatgtgtaatatcagttatctaaaatgttacagtttgttgcaaaatataagtaaaaatgaaaagtcttgaggatccataacccttaagatgtctgcaggcatagtattggatacatttgttcaagctggtttacctagtgaaggaggtcccaactaataaggcagtttcagccatctcttgtaacagattattctttgtgatgggtatacatttgttacataataataaggttaggatgctcatgtatagtatatacacaagcacttactattccacacagtttatttttgttttaattgagatagggtctcatcctgttgcccaggctctggtgcagcggtgcaatcgcagctcactgtagcctcgacctctcagcctcaagggatgttcccacctcagcatactgaatagctgggactacaggcatgtaccaccgtgtctggctagtttttaaaaatatttgtagagaagcgatctcactgtgttgcccaggctggtctcgaactcctgggctcaagcgatcttcccaccttagcctcccaaagtgctggaattataggtgtgagccaccgtgcctggtgtctacataatttcttcattagtaaaagacataactaatcttttccccctagggtaacttaagcaaaatgagagaattttctagcaaattgcaaatgctcagaagtcgttctgagacagtacatttaactctttaacagatatttatagagcaataagtatcactgcaatgatattgcagtgttctaggcagtattctaactagcagtccccagcctttttggcaccgagaccagttttgtggaagacaatttttccatgggcggaatggtttcagggtgaaactgttccacctcagaacatcaggcattagataaattctcataaggaacacacaacctagatcccttgcatgcccagttcacaaatgctgctaatctgacaggaggcagagctcaagcagtaatgctccctcccccgctgctcacctcctgctgtgtggcccaattcctaacaggccattgacacataatggtccgtggcctgaggggagttggggacctctgttctggatggtagataccaactaagaataaaaatgaaaacctctggccgggcgcggtggctcaagcctgtaatcccagcactttgggaggccgagacgggtggatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatacaaaattcttttctatttctggattccatactctggttctcctgaccaatttgactatccctttgttaagaatttattaattccactggtagagtgcacgttaattgggttttaactgttcaataaatatcttttttatttttattttttatttttatttttatttatttattttttttgagacggagtctggctctgtcgtccaggctggagtgcagtggccagatctcagctcactgcaagctccgcctcccgggtttacgccattctcctgcctcagcctcccgagtagctgggactacaggcgcccgccaccacgcccagctagttttttgtattttttatttttatttttatttatttattttttttgagacggagtctggctctgtcgtccaggctggagtgcagtggccggatctcagctcactgcaagctccgcctcccgggttcacgccattctcctgcctcagcctcccgagtggctgggactacaggcgcccgccacctcgcccggctagttttttgtattttttagtagagacggggtttcaccgtgttagccaggatggtctcaatctcctgacctcgtgatccgcccgtctcggcctcccaaagtgctgggattacaggcttgagccaccgcgcctggctattttttttattttttgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttgagatggagtctctgtcgtccaggctggagtgcagtggcgtgatctcggctcactgcaacctccacctcctgggttcaggtaattcttctgcctcagcctcctgagtagttgggattacgggcacccaccaccacacctggctaattttttgtgtttttagtagagacgagtttcaacatgttggccaggctgatctcgaactcctgaactcaagtgatccgtccacctcagcctcccaaagtgctgggattacaggcatgagccatcactcccggctaatcctgaaaatttcttacaggcacaagagtcaggtgagagtggacacagaagagatgcagagatagtacagacttttgttttttttgcggggaagggttttgcggaagaatggcatgataaccttcatctagcatcccttaatgttaccatcttatataaccatggtataattatcaaaattaagaaattaacactataggctttattccaatttttccactgataccttttttatattctaggatccaactcaggaaaataccacattgcatttcatcgtcgtctctttattctccttggttctgtgagatgtctttcttgaccttaacactttttttcttttttttagagataaggtctctgttgcccaggctggtgtgcagtggcacaatcattgttcacttcagcttcccaagtagctggaactacaggtgcacaccaccacacccagctaattttttcttattttttgtagagacggggcatatcactatgttgccctggctggtctcaaactactggcctcaagtgaatctctcacctcggccttccaaagcactgggattacaggtgtgagccactgcaccccgccgacactttggaagggtactgtttctccttttctccaaaatcaccatgacttattttttcttaacttactctgttggttagaagtaagtcaccaaggctagctcacacactcagaagaggaagattaagttacacttcctggagggaggtgtatccaaagaattcatagtcatttaaaaccattagttcgttggtcaaaacctttttttttttttttttttgagacagtctcgctccaggctgaagtgcggtggcatgatcttggcactgcaacctctgcctcccgggttcaagcgactctcctgccttggcctccctagtagctgaaactacaggcacgcgccaccatgcccggctaattttttgtattttttagtagagacggtgtttcactgttggccaggatggtcttgatctcctgacctgtgatctgcctcggcttcccaaagtgctggaattatagcaggcgtgagccactgcacctgggccaaaaccagtatttaataaatattttagaggtggtactttgaggctatgcagataccctgtttacccttttaaggtttcatcaactaatgtgagcttccatgggtggatcttcctatagaaattattactatggtatgttaatggtgattttctatttcactcattctttctacatttattaattaattctgtaagaaaaaactatttttcccttatccaggatccatttaaacattattcattgtatttggttatcagtgttttttatgtccttttttttttggtggggggacggggagggcagtgtcttgctctgtcaccctggctggagtactgtagtgcaatctcagctcactgcaatgtccacctcctgagttcaagtgattcttgtgcctcccgctcccagggagctgggattacaggcgtgcaccaccatgcctggctaatttttgtatttttagtagagatgaggtttcaccatgttggccaggctggtctctaactcctggtctcaagtgaaccgcctatctcagcctcccaaagtgctaggatttcaggcttgaaccactgtgcccaactttctgtttcttttacttatttgtatcttagaaatgaggtcttgctgtgttgctcaggctggattcaaacttctgggttcaagggatccacccacctcagcctctcaagtagctgggactgcgggcttgtgccaccgctcccttttgttcaagaacagtcatctgttgtgtttgttttaggacaataattttttgaagagactgagttgtcttataaaatgtcccacatctggatttgtggaattgttttatatgcccgatatcctataaacttaaatctaattttcagcttggttagatttaggttagacttttttatgtaaatattttataggtgatactgtgtaattcgtattatatatatgcagtgtctcattggttgttgcccatctataaaataattgaaaattttatcactgggttatagtggtaacagccaaagctctcatttttgaggtaaggttttccctttgcaatgaattaagtaatctatgtgataactctgacaccttgtgactatttccacaccaaccattcacgtagtaatttaagcatagattaaagatccttccctgaatcagttgtttcactagggtttacacattgttaatttaatactatctttctacatttattaactagcattcttttgtagaggagaacttttctaaactagagctattagattacactatttaggagttcctacttaataggataaatgcttaattctgtctctttttcagaatagagacttggtgcagtaataattcccaatattggtaaattgctgttgggtaaatgttgctgttactttctcttttgagtaccattattttacctgatctttaaatgtcatttgttattgactcagcatttcttcctttgccatcttaaccttttccgaaggcttcatgttaagtactcagtatgtgtttgttgaatggtagatggaaagaagaaactgaatccgcactttttgagcatgtatgaatatttatttttctgaggagaaagtccgtaccttgtatcaaattttctgaagggtacctgagagacaaatgagaaactaacgttttggggagactgcattctctgattccagaactggtaaacctctaactctgtaacatgactagaggtaaactcatctcaaaatctcattttaaaaacttatttataaaccctttattggccgggcacggtggctcacgcctgtaatctcaacactttgggaggccgaggcaggcggatcacgaggtcaggagatcaagaccttactggctaacatggtgaaaccccatctctactaaaaatacaaaaaattagccgggcatggtggcgggtgcctatagtcccagctactcaggaggctgaggcaggagaatggtgtgaacctacaaggcagagcttgcagtgagctgagatcgtgccaccgcactccagcgtgggtgacagagcgagactccatctcaaaaaaaaaaaaaaaccctttattttatttttagagatagagtcttgctctgtcacctggtgcagtggcataatcatagctcattgcagcctcttaactcctgggttcaagtgatcttcccacctcagtgtctgaagtagctgggaccatgccatgggacatagtgccaccatgtctggctaattttttatcgttatttttattttttgtagagacgggggtgtcactgtattgactgggctgatcttgaactcctggactaaagtgatcttcccaccttagcctccaaaagtgctgggattacaggtgtgagccactacccctagcctaagccctttaaatgaagatcatcacagtcttcggccttgtaatgcactatttatatttttattgattaccaactcataatctgtgatctttctactccctttcacccctcttctactgatcagtaatgttaaatggaaggtttctcatcccactgtgagcattcaaaacaactgataatttacctataactttttaaaaatgtgttcattgcataaccaaagtctacctaaattgaacaaataagggaaataatgttaatttaaaaattgggggtgccagcctagcctgaccaatatggtgaaaccccatctctaccaaaaatataaaaaaattagctgggcgtggtggcaggtgcctgtagtcccagctacttgggtggctgaggcaggagaatcacttgaacctgggaggcggaggttgcagtgatcagagatcacgccactgccctccagcctgggcaacagagcgagactttgttcaaaaaaaaaaaaaaaatgggagaacgtcagtgttcttagtaatattaataccatagaagtagaaataattgcagtgcttcttaggtttgattgtataaattttttttctttttttttttttttgagacgatatctcgctctgttgcccaggctggagggcagtggcaccatcttggctcaccgcaagctctgcctcctgggttcataccactctcctgcctcagcctcccaaataggtgggactacaggcgcccaccaccactcccggctaattttttgtatttttacagagatggggtttcaccgtgttagccaggatggtctcgatctcctgacctcgtgatctgcccgcctcagcctcccacagtgctgggattacaggcgtgagccaccgtagccagccttcttttttcttttttaagacagtcttgcttcgttacccaggctggagtgcagtggcgtgatccaggcttgttgcaacctccccctcctgggttccagcagttctcatgcctcagccacccccaagtagctgggattacaggcgggcaccaccatgcctagctaatttttgtgtttttagtagagaaggggtttcaccatgttggccaggctggtctcaaactcctggcctcaagtgatccacctgtgtcagcctcccaaagtgctgggattacaggcgtgagccactgcgcccagccattattgtataagatattttatataactaatgatgattcatttgagtgtttttaaagtttatcttcctaaggtaagcctggaaacttagaaacaagcaataggtttaatttgcaaaaccatgactaaaacagacatgcacatactagcttccccttcactagcaaggtgtaacatatttcttttctttttttttttgaaacatagtctcactctgttgcccaggctggtgtacagtggtgcaacctccgctcacccagcctccgcctcctgggttcaagtgattctcctgcctcagcttcccaagtagctgggactacaggcatccgccaccatgcccggctaatttttttttcgtgttttagtagagacggggtttcaccgtgtcagccagggtggtctcgatctcctgacctcgtgatccgcccgtctcggcctcccaaaatgctgggattacaggcttgagccgctgcgcccggcctcaaagagatttttaagtgttggcaaacttttaaaagtcactcccaaatattatagtataaggcaaagatctgcctatttctcaagtttaatttttaataaacagctttagaaactggtggagaataagcaaatttaaaatggtaaatcgagggctgggtacaatgccttatgcctgtaatctttaagattacactttgggaggccgaggcaggcagatcactcgaacccaggagtttgagaccagctcagcaacatggtgaaaccccttctttacagaaaatgtaaaaaaattagccagatatgatggcacacgcctgtggtcccagctacttgggtggctgaggtgggagaatcccttgagcccaggaggtggaagttgtagtgagctgagattgctccgctacactccagtctgggcaacccagcaagaccttgtctcaaaaaaaaatagataaattgaggtaaattgagctggatgtggtggttcctgtagtccattactcaggaggccgaggcaggaggtttgcttgaggccaggagtttgagagcagcctgagcaatatagccagaccatttctcttaaaaaaaaaaaaaaaaaaaaaagccaagtgcagtggctcacacctgtaatcccagcactttgggaggctgaagtgggtggatcacctgaggtcaggagttggagaccagcctgaccaacatggagaaaccctgtctctactaaaaatataaaattagctgagtgtggtcgcgcatgcctgtaatcccagctactcggggaggctgaggcaggagaatcacttgaaccctggaggtggaggttgcggtgagccgagatcgagatcgcgccgctgcactctgtcctgggcaacaagagcgaaactccatctcaaaaaaaaaaaaaggtcaattgtttttctttgtgtttcagatacgtcggtaacctttccagagatgtgacagaagctctaattctgcaactctttagccagattggaccttgtaaaaactgcaaaatgattatggatgtaagggtattttacttagttaactttattcttttttttttatttttaagatgttacttaaagcaattgcttggaaggaattcttaatttaccatagtctaaatatagtataaattcttagtttactgtagtctaaatatagtcttagtttactatattctaaattaatagggatttggattttagtgaatttgtaatgaacttgttttgacagctaaatttaatttgttttactaaataatggaagatgagaatagaactagttatttgtagatcagatgggaactctaaaaatgtgggctctttcaggtgcattgaggcatcagaaatatttatatgtggtgtcaattggttggcaataaattcagctcttacaattaaattgggaaacaggtttaaaaataataggcatttgatacagtgaaagtatttttaactttatttttaaaggtcatgattcaagtttttcttgtatttaggcaattcttatttatgagaccttaatgtattaaaaaaacacacacattgaactcattcatccaattggtgttaaccctgattcataatatgtataaaaagacatttagggaacagttaccctatttccctggagtctttgtctcctggggaggaaaaatacttttttttttttttttttttgagacggagttttgctcttgttgcccaggctggagtgcaatggcatgatcttggctcactacaacctctacctcccaggttcaagcaattcttctgtctcagcctcccaagtagctcggattacaggcatgtgccgccacacctggctaattttgtatttttagtagagatgggtttctccatgttggtcaggctggtctcaaactcccgacttcaggtgatccacctgtcttggcctcccaaagtgctgggattacaggcatgagccaccgcacccggcctaaattttttaatgggaaaggcaatgttaaagagaaatccttttgtcccatggtctctcctttctgtaaacttttgggttttttgttcgtttgtttgtcttgaaagacagggtcttggtaatagccactgcacccagcctgctataaactttcagttttcttttctccagaaaagacaatgtatatgtgtatctgttaccttctttcctgttgtattccagctaaacttttttttacttggtatttttaaaagtaattatttagccgggtacagtggctcacacctgagaccacatctctaaaaaaaaaaattaagtgaaaagtattaattgtacttgaaataaaattgtatattcctctattttggtagctttctagtcttttgaattagttttttattttaacaaatctgtttaggaggttatggaaagaatttttaaatggcctataatgtcatttggtattttgcactatagatttaaaaattggaattttttcctgctactttaatttataattaaacatccaggttctacttatcacatagcctcacctacctggaacaccagcagaaaagggaagatactttttttctttctttttgagatgaaatctcgctgtgtgcccagactggaatccagtggcacaatctcagctcactgcaacgtccacctcctgggttcaagtgaatctcctgccttagcctcctgagtagctgggattacaagtgcacgccaccacacccagctaatttttgtatttttagtagaggtgaggtttcaccattttggccaggctggtcttgaactcctgacctcaggtgatccgcctgcctcatcctcccaaagtgctgaggttacaggcatgacctactgtgcctgacaaagggaagtttttttttttttagacggagtttcactcttgttgcccaggctagagtgcagtggtgccatcttggctcactgcagcctccaactcctgggttcaagcgattctcctgcctcagcctcccgagtagctgggattacaggcatgcaccaccacgcccggctaattttgtatttttagtagagacagggtttctccatgttggtcacgctggtcttgaactcctggacctcaggtgatccacccaccttggcctcccaaagtgctgggatcacaggtgtcagccatcacgcctggccaggaagataattttttaaagacttctgaacaaggaaaaaattgatgtcagaaagttcacgttctaaaacctattctaaaattttacttaccagagaatctcttcagttttcagagcctatttacctgtattttaaaacaggtttaagaaggttggctaagattttctgctactcagcaagctagtagcagcaaggagctatgtgcatttgtagcagtaaaaagtaacatgtaacagggaggcgatagaaaatcataaaaagccgaagtgagacttcagaacctaggagttcagtcaaacttcatctgtgtagcttgaacaaatggttttatgatgacagggtatttatagctatatatccagttaggatacctagtcattaatactaaattatagtactatagtataacctgttgaagaaaatagctacatataaaagcagtgtgattttaagtgtttgctttttttttttttactttttgattcttttagagacaggatcttgctgtgttgcccaggctggattcaaactcctgggctcaagcattcctcccacctcagcctcctaaagtagctgtgactacaggagctctccaccaggcctagctgtttactgtttgttttgttttgttttgttttgttttgttttgtttttttgagatggagtcttgctctgttgcccaggctggagtgcaatggcgtggtctccgctcactgcaacctccacctcctgggttcaagcaattctcctgcctcagcctcccaagtaggtaggattacagaagcctgccaccacgcctggctaatttttgtatttttttttgatagagatggggtttcaccatgttggccagtgtggtctcaaactgctgacctcatgatctgcctgccttggcctcccaaagtgctaggattataggcgtgagccacctcacctggcctacttttttaaatctaaaatgtacttgcggaatggaccattcattctccatggcctgataacttagatgtataaaacttggtttaagatttttttttttttttttacataattttaaggtaatgggtgaataaagcttaaaattggcttttgtgtttcttctcccacagacagctggaaatgatccctattgttttgtggagtttcatgagcatcgtcatgcagccgcagcattagctgctatgaatggacggaagataatgggtaaggtaagctgtcgtaattaaagaaggtgacttgcactgacagaatttaagttacatatgattttgtttactttttggaaacattaaacacctttttttttttttttttttgaaacagaagtttgcccttgttgcccaatctggagtgcagtggcacaatctcagctcactgcaacctctgcctcccaggttcaagtgattctcctgcctcagcctccctagtagttgagattacaggcgcgcaccaacacacctggctaattttttgtatttttagtagaagcggggtttcaccttgttagccagactggtctcaaactgctcacctcaggtgatctgcctgctttggcctcccaaagtattgggattacaggtgtgagccaccgcgcctggctgaaacattaaacactttatagcaaaaggtcagttattcacatataatattaaaaaggtgagataaagcttttagaggccagaggcctgtgtttatgttagtattttctttttttttttttttttttgaggcagagtctcactctgttgcccaggctggagtgcagtggcaccatttcggctcactgcaagctctgcctcctgggttcacgccattctgctttggcctcccgaatagctgggactacaggcacctgccaccacgcccagctaattttttgtatttttagtagagacggggtttcatcttgttggccaagatggtcttaatcgcctgaccgcatgatctgcctgtctcggcctcacggtggcgtaagctactgtgcctggcttttttttgagatagaatctggctctgtcacccaggctggagtgcagtggcacaatcttggctcactgcaacctccgcctcctgggttcaagcagttcttctgctttagcctcctcctggatagctgggattacaggcacccaccatcacgcccagctaatttttgtatttttgtagagatagggtttcactatgatgaccaggttggtcttgaactccttacctcaggtgatccacctgtcttggcctcccgaagtgctgagattacgggcatgagccagcagcctatattagtattttccatggtggcttgtacatggtaaatatcaaaatatttgctgaataactcaagcttggatgctaatggaaaagaatgaaagggaaaaaaatgtgtgtactttaggtaagaaaggttaattgaatgttaacagacatacaaagattaggatttgtcaacattatttccctaaatataggtcagaagattaagtattgaaacttgaccagagctggtatctccttcagtgtttcatcttaaacagatatcttcctatctttttatagatgtaaatgtgattttgggttttatttttgtgaagtataaatgtagaaatttttttttaggaagtcaaagtgaattgggcaacaacccccagcagtcaaaagaaagatacaagcagtaagtatattttatgctctttgagcatttgtttttattatacccagtagttttattgtaaagcctataaacatcatacatgtttgcagtaatgttttgatcgttatcctaagatatgattgaatgtgtttgtgttaataaatttaagaacaaatctaatctttgtcatcaggtagtaccgttgtcagcacacagcgttcacaaggtaattgtatcttcttaaacataaaatgaaatctcttgaaagggtattcactaaccacctgaggtttttttgtttgatatttgggggagggggggtgggaggagatattatttctatttgtctctttagcaatatttttctcctttctcggtgttgaccaagtatagcttgtctgctactttggtgctagttcaagctttctgatccctttggtggcagctgatgccttagaactactttcaccaactaaggggcaaaatataccctcctttcttgaggtcgccatctgggcttcatacccagatcttccaaatgcttgagttgctcctcaaattttgtttcccaaagagtattccaaaatgttgtttaaggcctgtcaaatatgggtaatttttctttccaaatatgctttgtcaaattgatgtatgtgtccattttaaagtgttggtccaacaattttgcatttttaaagtgtttctttttttgataattgtcttttttaaaaacttcagatatgggatggttatttctctccaatgcttttttaatggttctgatataaagtgaagggattactgttttcattctgttgccttcagtcttagttcacttgcacatggattcacataaactgaatggtgtaatgtctgggcaaccaaaactgttggcttttgagaaaactgtcaaatactttaacatcaaactgttgcaatgcaaggtatttctttgattgttcttcacaaaatatggttaaaccaagtatatatcatgtagctagcttcagtaaattgtgttaactgaggcaaatctagtctacataattcacagtaccactattttgttttaatttgtaaagccttaatatagtggtaaactgaataaaagtaaataattattattagaatggtaactaagtcattaaatttttttgcagaactgaaacttgtatattattagtttattttcttagaccagtataattgactgtaaatagaaatataaatgtcactttacagttagatgtatcacagtcgtttcagaatttttcctatattgttaccttgattcattgtttaaaattgataggatttgtatagatataggatagtgttttatttatactttatcataagtcataatcattttaagaatactttattggatagattttagtactttttaaattctaaagttctatttttcttttcacttccccttccttccccttataagatcatttccatgtctttgttggtgatctcagtccagaaattacaactgaagatataaaagctgcttttgcaccatttggaagaatatcgtaagtaacagaagataaataaaatccttttaattagaaacaattatatgtagacataacttgaaaataattgtgaattttgataaaagtgataaaacattaattttatcacttgaatataatgtattacattgtttagagatcattcaagaagttaacagccaaagattgttttcccattgatgaataaacctttggttttcaattatcttaaaatcctttttcaattagcatagctcttaagagacaaatatgccattgtacattttatagtttattatataggtatcaagctagcttcgtgacagtctttaataaaattgtgctgtaactaggaatttagatgtgttgggcacttctatgataccatgcattttgtgttaatatattttaacaaaaagaagccttaacaattcaacttaaaaagcagttgttgaatattatttcctagatatttatacccttttaaaaaaactgctaacaataatagtgccttaataatattttatgaagcactgccttgcctgcacttgttattaatcacacattttaggcccttatgaataaataccttacttaagatagcattcatcatagaagaactttaaaattttgaaaagcatgccatattagagtaagaataacaagttgtttatagtactttagagtaaatagtttagaaaacttcttcaggatttctggttgctttattattttccacctagaagaatagtaaaaaggcaatgctagctgtggtttggtttgaatagaccaacagcagagatgagatgaggagactggaaagtaaatgccagtagtattggtgattaattttgggagctttcatttcaagtgttacacttcgttgattaggggatgacagtgcatgtgaccaagggattaccaactttaaaggtggtcacagaattagggcttgtaaatggacttctttcagatgaaatttttctttgtgagacctgatgaggaaaataagtacagctttcatttcttgattgtccctcctaataatatcttagagtttcaataataattagcttgcttttgaatatctggacttagacagccttagcctttggcctctcaagagctctaaatctctatgcaaaatttggtgttttcattttggtgtggaaaatctttgtaaattttatcctattgtcagagtagtcacaggaccataaaaggataaagaagcctgatctagatctttgttgctgcttctctagagaatcttttcagagagaaataaatatgagttgaatatttttcaaggttaacattttctgaaactttaacaagaaaaaattttctgacattgtgaacagattaagtagctattttattaaatgctttgattttgattgaggtagttttttttaaaacatgggaattttagtttggaattaggcaaagcttgtttaacagttggtgagtttaagtgaccttcaaattttagcaatttcatgatttacaaatcatgggttataattttttctcttaaatatatttctgtatcatatatttagtttgtagattttcttatggtcccatgattattggtgtatgctttttttttttcttggtaagacctttaagtttcatgattttcaattgagataatgtataatgtttgagttttaggctatataaggagttagctttcctacatttaaaggaatcatttggcagtcagtagcatattttcctgattttttgtttttaaaggagataagttgataactatatattgaagggttctcatgatattctctaaaaagtgcaagtatcagtgcttgatttatagatgtgtaataaacagtggtctgtaaaagcatttagtataaaaaatatggtgctttcccttatatacagcctataaactcttcctgttccttgagtttaagggtagactaacaaattacagtgtagaagagagtcagctaccaaattaaatgcatgcgtaccatacagataatttattaagtatttttacttattgagcattgacttacattagttttcccttttcaccaccacgtctttgtcattttgaccctgaacacttaataaatcactcagattgatctaaaatatgcttatgtatactttaatagaaaattacattgcatggctttctgacttgggtttttgttataaaagtgcctgttttgttcatgtgtcctgagagagcaagcattgtgacatacctgactaacttaaaagcagattgcctgtgaagcacaatttgagtccaattttttgaggtatggagttttagctatcatggctgggtttttactcaatctcaaataatagggctctggttattttgcagagtgtctctgaagaatggacagaattgccctggctaactacaagctacggttcacagtggataaatgttggcgtgcttttttactttctgactttttaaaattttgcttttatatcttgtagttccaatcaattttatatgaatgctattttaaaattcagtgtaaaatcttttcttgtgtagcttaaaacgtgtgtgtgtctagtatttttcttcaatgatttaacatttctgaaaatgtcaagttctcaaaatttagacaaaaaggggggtataaatttatctgaaaaaatgaaatagtttaattagtttagggaatatacaactttttcattcttttggtttccagttttctttttttttttttaatgattaggtagaatatagtgtcaaataagttataaattgcatggttaaagaaagccattcatctaatgtcctgtgaactttataagtggttaagtagttcatgtgagtagctgctttcaccatgcttaatatttaaaagtttaaaaagtgttttgaagagagaggcaatttctgtacaatataaataaacatatatacttaagttattgatttgcatgtctataaaattaagcctcatttcctaatacatgataaattttgtagaataaaaggtgaatttgcattaaaggttcactttaatgaaggcgaaacatgagaatgagatgtgtgtgaaattctcatttgatatccagaggacagtttgcaaacctaaacttctgattgtttctgatttcagagatgcccgagtggtaaaagacatggcaacaggaaagtctaagggatatggctttgtctcctttttcaacaaatgggtgagctcaatcagaagtgcatggacaatgcttaaagagtaaagaatggaagcgctcagttttaattattaatctttttaatctgttgtttatttaccctcactagaagatatcttcttctgttttgtctatttaacatttattggcacctgagtactggcatgcagagtactgatgttttcagaatgtagacagtaaagttgtttaaattatcagccaaaggtagatgttgaaaatctcatttatggatttaattcattatctccctagtttatattcccatggtagcttagaagtagctaataaaatactgtgaggaggccaggcgcagtggctcacacctgtaatcccagcactttgggagacagaggtgggtggatcacctgaggtcaggactttgagaccagcctggtcaatatggtaaaactctgcctctactaaaaatacaaaaattatccgggtgtggtagcaggcgcctgtagtcccagctactcaggaagctgaggctggaggatcacttgaacccaggaggcagaggttgcagtgagctgagatcatgccacagcacagcctgggtgacagagtgagaccctgtctcaaaaaataagtaaataaaataaaatactgtgatgggtaggaactttttaaaaaattatatattacccaggtacagtggtttatgcctataacctcagcactttgggaggctgaggagtttgagatagaccctggcaatatagtgagaccctatctctacaaaatatatatatttttttattttactttatttaatttatttattattattattttttttttttcggaaattccgaaaattcagccgagcccagtggctcacgcctataatcccagcactttgggaggccaaggtgggtggatcatttgaggtcaggagttcaagaccaacctggacaacatggcaaaaccccatctctacaaaaaattagccaggcatggcgacgcatgcctgtagtcccagctacttgcggggctgaggcgggaggatcacttaaacccaggaggtcaaagctgcagtgagtcgtgatgaagccactgcactccagcctgggtgataaagtgagaccctgtctcaagaaaaaaaaaaaaacagttgaaaacggtaaagtatattgagaggtgaaaacaatacaaggtcaatcatgtacatgtcccatctaccaaagatttttttttttttttcctgtaggatgagaaaataaaaattaaagagaatttatttggttctgaaaattgtactattaatcatagcataatattttagaaaggtagaaaaatactttctataatggattaatagccatgattaatttgagttccttagaggattgattcataggttttaggatgttttagtgcaacttagggaaataaattgggggtttggtgggctaggaactcgtttttcacattttcaagattgtggactataggattctgctatataaaaatcactattgaatgtgtatgttgtatgaactagagtcaaatagcaaaggatgtctattgatacctgtgtatgacctctggagttagccagaggtgtatacaaattctgacaaccattttaaaacagatgtaactttgagtaaataatataatcttctataaaatcttctataaatttcaacttacgtaaaatttgtgatatgtgcttggctgtgttatgcagtgctacagccctgattgcagttacccccagcctctcatcctgtgttctacgtgctaggaaaaccacaatactgattgagcttagcttctctgcctaccccaaaataactgaatgttgctggaaaaaaactgagttcaagtggttttgctcctaatttaatggtcacacactttagatgagtacagagtactgtcttggcaaacatgtttctcatctttcaaccaccaaatttacataaaattcatccaccttcccattcttcctcctattatagtacaggaaaggtccttcctgtcaaaggcaaaatcacttatgattgtgtccccacctctcttgccttttcaaggactttgagcttatgctgccactgcctcatttttttttttttttttaacagcagctttattgagatataattcaagtatacagtgtggttgggtgcagtggctcacacctgtaatcccagtactttgggagactaaggcaggcagattacttgagcccaagagtttgagagcagcctaggcaaaatggcaaaaccccttctctataaaaaatgagccagacatggtggtgcacacctgtagtcccagttacttgagaagcataggtgggggatcacctgaccccaggaagttgaggatccagtgagctgagattgcgccactgtctcaaaaaaaaaaaaaaaatttattttttttttaattatacaattcagtggtttctagataattcacagaattgtgcaactatcaccaaaataaattttagaatatttttaccaccccaaaaagaagctacatgactcattagcagtcactatccttttcctttcatgcccctcctgccagccgtgaacaattgtcagtctactttgtgtctctagatttccctgttctcaacatttcctataaatggaatcataatatgtgctgttcttgttgttactactatcctgtgtcatcactctattccttctctactggatcattctcgtcaaccaaaaaacacctaggatctctctctctctttttttttttttggacagagtctcactctgtcactcaggctggagtgcagtggtgtgatcatagctcactgcatttttgaactcttgggtgcaagccatcctcctgcctcagcctctggagtagctgggaacacaggtgtacaccaccatgcccagctaatttttttttaaaaaaaaaggcctggctcaatggctcacacctgtaatcccagcactttgggaggccgaggtgagcaactcatgaggtccggagttcaagaccagcctgatcaacatggtgaaaccctatctctactaaaaacagaaaaattagccaggcatgttggcatgtgcctgtaatcccagctgcttaggaggctgaggcaggagaatcgcttgaacccaagaggcagaggttgcagtgagccgagattatgccacagcactccagcccaggcgacagagcgagactccatctcaaaaaaaaaaaatttttttttttgtagtaacagtctccctttgtcgcccaggctgtcctcgaactcctggcttccaccagtccccccacctcggcctcccaaagtggtggggttataggcatgaggcaccgttcccggccatctaggatctcttaatcaaaaccactgtttatcttcacatcccttccctacctatatccccatttctcagctataattctcaagagttgtttgcatatgcttcccttttatctcccattcacaaccatcaacatagatacatctatgttttttctccacttttttgagtgagtagttttatttttttcctgaggagctgtatcaaagtatcataaactggatgacttagagcaacaaaaatttattttctcaatgttttggaggcatgaaattcacaattaaggtgtcagcatgtccatgctctcttgaaggctctgtggaagaatcctttgttgcttcttccaagcttctggtggttgctggtaatctttggcattcattggcttgtagctgtataactccaatctctgccttcatcttcacatgaccatcttctcttgtgtatccatataatatcttttttttttttttcttttgagacagggcctcactctgtctgtcacccaggctagagtgtggtggcacaatcacaactcactgcagcctcaacctcccaggcttaggtgatcttcccatctcaacctcctaagtagctgggactgcaggtgtgcaccaccatgcccagctgatttttctattttttgtagagacaggatttcgtcatgttgtccaggctggtcttgaactcctgggctcaagtgatccacctgcctcagcctcccaaagtgctgggattataggtgtgagccaccatgcccaatctaatctttactttttataaggacttggatttggggcccaccttaatccaatataacctcatcttaattacttctgcaaagaccctgtttccaaataaggccacatttcacagataccagggtgtaggacttcatatcattttgagggacacaattcaacacataacgttagttaagcatctcatggagttcggacaggttagtatgataattgtttctttttaagaatgtacatttcctgccaggcacagtggctcatgcctgtaataacagaactttgggaggccaaggcaggaggactgcttgatcccaggagttagagaccaaccagcctgggcaacatagcaataccctgtctccattaaattaaaaaaaaaaaatgcctgggaacctagtatgtggacaccgaatcccctcccattgcctgttgcaaccagtaaaggccgttcttaccatagtaaaaaaaaaaaaagccagctgtgtcggtgcatgcttatagtcctagctactaggaggctaaggtgggagtatcacttgagcccaaggagttcaaggctacaataagctatggtcatgcctggatgacagaataagaccctgtttaatttttttttaaagagtttccttttcgtatctcaagaaaggccttgaatttgcctgttccttcagtagtagattaaacttacctttgatgtgtgcacacaaaagttataccccatttactttttagtctactctaagctggtttcactctcattttctaccaatgctactgtattttcaaggtcacctcttaacctctgttttcttggctcccttgaccatacacgtttgtggcttcttccttcatcactggctactcctccctagtcaccttagctagtgattattcctttacccatttttcaagttttgctgttttttgttttgttttgttttgttttgttttgtttgagacggagtctcacgctgttgcccaggctggagtgcagtggcgcgatctcggctcactgcaagctccgcctcccgggttcccgccattctcctgcctcagcctcctgagtagctgggactacaggcgcccgccaccgcgcccggctaattttttgtatttttagtagagacagggtttcactgtggtctcgatctcctgaccttgtgatccgcccgcctcggcctcccaaagtgctgggattacaggcttgagccaccgcgcccggcctcaagttttgctgtttttaagggctctatcctaggctctttctctagtctgcatgaattttgtgtcttcagtctacttctctgtgttccacacttactaacaaatggtcttcatttatatgtctcacaggcatctcaaagtttttttttttttttttttttttttttttttnnnnnnnnnnnnnnnnnnnnnnnnnnnnnnnnnnnnnnnnnnnnnnnnnnnnnnnnnnnnnnnnnnnnnnnnnnnnnnagacggagtctcgctctgtagcccaggctggagtgcagtggccggatctcagctcactgcacccgagtagctgggactacaggcgcccgccacctcgcccggctagttttttgtatttcttagtagagacggggtttcaccgtgttatccaggatggtctcgatctcctgacctcgtgatccacccgtctcggcctcccaaagtgctgggattacaggcttgagccaccgcgcccggcctcaaagttttttcttttttttcttttttcttttttttttttttttttttttttttgagacggggtcttgctctgtcacccaggctggagtgcagtggccggatctcagctcactgcaagctccgcctcccgggttcacgccattctcctgcctcagcctcccgagtagctgggactacaggcacccgccacctcgcccggctagttttttgtattttttagtagagatggggtttcaccgtgttagccaggatggtctcgatctcctgaccttgtgatccgcccgtctcggcctcccaaagtgctgggattacaggcttgagccaccgcgcccggcctcaaagttttttcttaatggagctttttgattcttcctcagacctgttccccttttctttactagtctttctccccatttcaataaatgctgtctttccaatttctctagaggaacctaagagttatcttcgatttctctccttttgcttgtggcatctcctccagcccccattattttggccctgagtttaggttacattgactgttactgcaatgctgctggccaccaacactttttaaaaatctagaatattgcacaagtttcataaaatatctttatcctcttactacccattacccgaagccattctcctcttagcactaagagtggccttcttaaaacataggtcatgatgattccttgcttaaaatctttcagtttcattctctgacattctgttttgctcactagattccactgaaactagtctttcacatgcttctttcagatgcataccaaactcctcaagccttaacacactttgcaatagttttctctccactggggctttcttctccattgttttgacttggctatctcctattgctctcaggctcaatttaaatgtcatttcagaaagacctttcctatttatcctgtctgtagtagatccttgtgcccatattctcgtatctcagccttttgtttctgtcataggtcttagcacaatttgtaattgctttatttgttccccacaaaagaatgtaagagaccatatatgcttgtttaccagtgtgtccctagctcttaactgtgtgcctggaacataactgttcaatatttatttgttggatgaatgagaaaccaaatattgttaacaagcttgttagattgtttaaagtaagattttcttctataatagtctgagttgcatgaactttatgatatctacctcattagggcttttggactcaggaccttttcagatgattaacgttcccacagtttaatattcaaataagagaagaataatttaaaagcaccattgggttgagcacgctggcttatgcctgtaatcctagcactttgggaagctgaggtggacggatcacttgagatcagaagttcgagaccagggccgggcgcagtggctcactcctataatcccagcactttgggaggccaaggtgggaggatcacgaggtcaggagattgagaccatcctggctaacacggtgaaaccccatctctactaaaaatacaaaaaattagctgggcatggtggcgggcgcctgtagtcccagctacttgggagactgagacaggagaatggcatgaacccgggaggcgaagcttgcagtgagccgagattgtaccactgccctccagcctgggccacagagcgagactttaaaaaaaaaaaaaaagaagaagaagttcaagaccagcctggccaatatggcaaaaccccatctctactacaaatacaaaaattagccaggcatggtggcggatgcctgtaatcccagctactcaggaggctgaggcaggagaatcacttgaacccagttggcagaggttgcagtgagccaagatcagcctgggtgagagagtgagactccatctcaaaaagtaaataaaataaataatttaaaaatatgaaaaacaccactgcattttacgaaagctatcatatattgtgtattttgtaaagcattttcacttaaacatttgtggaaagcaccaagttttctctactgctaaaagttgtttaacatcaactaatagtgacttctttattgtttctaatgtgcggaacaggatgctgaaaacgccattcaacagatgggtggccagtggcttggtggaagacaaatcagaactaactgggcaacccgaaagcctcccgctccaaagagtacatatgagtgtaggtgtattggagaagaaaaggaaatgtggaattttggagaaaaatacactagattttaaatgttagagctgttcccggagacttgttgcagaaatagatgagaagcaaatcaagactactgttcaaaaatgtacttagttttcatttttgtaattataaatgatattatttctaatgtcaagtctcctattaagtagaaaatactgggtaattttttagacattcgtgggggaggtttaatcctgaactgaagtataatatgtataatataagcattttgatcaaggttaatgttacctttacagtagtgtagtgaatattgcttttctctttctgtacattttatttaatgaattaaagacataaaccttttctctcaccagcaaataccaaacagctatcatatgatgaggttgtaaatcagtctagtccaagcaactgtactgtatactgtggaggtgttacttctgggctaacaggtatgggagccttccctgggtggcattaatttttaaagtgcaaagcttaatatttgtaggatttttatataacttagtgtgatttttgtttttttgtctaacagaacaactgatgcgtcagactttttcaccatttggacaaataatggaaattcgagtctttccagataaaggatattcgtttgttcggtagggatggttttttaaaaaaaacttttttcttggtaatgtaactggaaaaccagaaaataaataaaaataaaaaataaactttttcctctgaaatattttttggatatttaccttgttttcttaaaaatttaccttggttataaagaagttgaaatttcggccaggtgcagtggctcatgcctgtattcccagcactttgggaggccgagacgggtggatcaccgtcatgagtttgagaccagcctggccaacatagtgaaaccccttctctactaaaaatacagaaatacaaaaattagccgggcgtggtggcacatgcctgtaatcacagctactcaggaggctgaggcaggagaatcatttgaacccaggaggtggaggttgcagtgagccgagatcacaatcactgcactccagcatgggcaacagagccagactccatctcaaaaaaaaaaaaaaaaaaattgaaatttctctagcatctagcatatttgcctctgtgattttatagccccctccctaacacaaggccagtgaaacacactttctcaattaacacaagtttccccagaactacaagatctttgaaaaaaatagcaaagaaaatacaccatgttatgcaaacttttacctcagactcaccttgatttttaaaaattggctttattcataaatatttaaaagaatttgtattggtgattcttaatattgttttcaataggttcaattcccatgaaagtgcagcacatgcaattgtttctgttaatggtactaccattgaaggtcatgttgtgaaatgctattggggcaaagaaactcttgatatgataaatcccgtgcaacaggtgagagggttcttaactttgagaagtaattgttgggctaataatgttattttaaagtgaagattgttgataatccttgttaataagtttctagctaaaatgatagtatataagggctcttcctacatgtattatagttccagaatggtgtttcatggattgcagtggtgacatgatgaatgtgaccgcctcagttgactcatgctcatacagtggattttaaaatgctccgagaaacctaaaagggtattaaatctttttttaatacttggtagatttattcacatactatttggagaactcacaagcatgcttgactcaactgaaacaaaaataaagctgtaaatatagttagggccttggcatttttcttccagattttgctgccttttttttttttccaggcagctttagtggttcagacttgattccacaaagatttctatgaggaaactgtaaatggagcacagagatgggagtaagcaaatcctaaactatataagtttcttaagtaagctgaatatagaaataactaatcacatcttccctatctaaaggaaacaatcatgtggatagtataccaaacacttaaacattctactatgtttaacatttattactaggatatcaactgatgccttccaaaaactatgccattcagagtatcatgtagagttacagcagtataacctcagatttttttgttttgttttttgttttgttttttagccgtcaactcttgtttatcctgtcattttaattgtaatgatggccctgtgtgtttttgctaagaatttgtggtatcaacatattaggattgtatatttacagttaacttcaagtaatactaaattttatttaatagcagaatcaaattggatatccacaaccttatggccagtggggtcagtggtatggaaatgcacaacaaattggccagtatatgcctaatggttggcaagttcctgcatatggaatgtatggccaggcatggaaccagcaaggatttaagtaagcatatctgatctcttcctttactaacctttgaaaatgtctaaaaatattttaagtaggaagaaattttaagaattagtggataatagggcctcttacataaagcgtaactatagccttcaaagaacttaattgttttttagtaagaaaataagagcttaatagttgttaaggttttcacatgactaatggtcacttagtcattcttatatacactgactttgatgctagttgttgtattcattatgcttttttctgtgcaaagggttttttcatgcattgttatattgcaagcttccttatacaccctaaattacattattttacttacatttctgaaaaacacactgaaaataaaaatacttgggaggttaaaaaggaatgtagtgggccgggcgtggtggttcatgcctgcaattccagcattttgggaggctgaagtaggcagatcacttgaggctagcagtttgagaccagcctggccagcatggtgaaaccccacttctgctaaaaatacaaaaattagccaggtgtagtggtgtacactaccaataggttccagctatttgggaggctgaggcaggagaatcacttcaacctgggaggtggaggttgcagtgagctgagaccatgccactgcactccagcctgggcaacagagcaagactctgtctccaaaagaaaagaattttgtggtgggaattttgtggttgggcacggtggctcatgcctgtaatcctagcactttggaaggccagggcaggtggattgagtccaggagtttaagatcagcctgggcaacatggcgaaatctcgtctctacaaaaagtacaaaaaaaaattagccaggcatggtggtgcgtgcctataatcccagctgttcaggagactgaggtgggagaatcgcttgagcctggggagatataggttgcagtgagctgagatcatatcactgtactccagcctgggtgacagaatgaaaacctgtttcaaaaaagaaaaaaaaagaaaaaatcttgtaatgttatgaatttttatgagcttatatttgttaataattgttttgatttcttctttgagtagtataatgttagctcttttttcattgtggggaacctacctgactctttcaggtagtggcttcctcagcaaatatgttcaaccaagatacagagatgagatggacagttttcttttccgagaatggggttaagtttttctgtgttcaggggagatgaatgttgataagttcagggtattttcttagctttaatttctaacaaaagtaaactgtccaagaaaaacatacagggatgcttgtttttaagagaaaaaaaactttagtagaagctttcgttttccctggcgtaagttattctatttgggtttaagaaaaagtatgtttcccatttcccatatttgttatttgttcttgccaatactttattttaatcagtggatttcctctcccctctgcttctttcacattctccaccagtcagacacagtcttctgcaccatggatgggaccaaattatggagtgcaaccgcctcaagggcaaaatggcagcatgttgcccagtcagccttctgggtatcgagtggcagggtatgaaacccagtgaaaaaggactccagaatctaaagccagtggcttgaggctacagggagtgtagtaaagccgttgtttacttaaagatttatcaaatcagtcagtgcaaatgtcagatacaatgtatttatttaaaagattcatttttaatcatgaaattacttatcatccacattgttttaaaaagaaacaagatgctggatgtctgccaatttttgccttcattaccttttttgataaagtttctcagatccttgtttcaaacacaaatgcagggattgctgccactttttaactattaagaggcagaaaattgcacaatattgaacttttttccactaaagtagtgtgcagttatagtttgcattcctgatatgatttaaaacatgtaatataaagatgttaaaaaaaaaaaactgtgcagagtctagaagttctctgtcatcttcagcttgtgcacaattctgttttaggttaaaaaaaaaggcattgtttgagctgtcccatctccactgttatccctttgtggttttttaatataaattattagtttatgtcatttttgtatctacatcttttttcacaaatttgtcttgccttattaaagttctgtaaaatatacttaaatggaaaaaatgatgttcatttagattgaaaacttttctcagatggattgataattgcattcatcttgtgttttatatgagaaggtgcctcaagaattccctgttggatttgtttaaaagaatttttatcttctgtgataaactttgctgtgtaccaggaactataaaaacaaaaacttgttactaaagaaaatatctgaaatgtgataagttcttatgccatgttaatttcatgtgtcaacttcaacatttacatgtattatttcattatgtaaaatgttttagcaatttaatattttgcacagttagcaaactttgtatgtcatttccttcaaggtatcatgcagagtttacatgaaatttataaggttttaagttgtttgcatgtgaaaatcaaatacatactttggtagtctttgaatacaaagtcatctgctcttgtttttcaagaattttgagacacaaagttgtatgtaaaggaatatatttgccgttttataggtagatttgctcaaaaagagtgaatcaacttaacatgtacaaatgatagctgtgaaactgtagaatatctatgtgtcaggcttggagttcattgtgacctccaaattttgcctgaaggaccagctgggcaaagcattttttaaatgttcagaggccaaaagataaacaaacaaaaaaaaaaacttaaaatcctacctctttaaacagccttcaaataagaatcctcagtgcaatcattattttgattcttttggtacctgttttcctggagttcccaattttattattttggagtggctccaagcattaagaggtttaatctttgatggcattgttctagttttgaaatttctattgtatttcagagtctcttagaaaacttgtgtgggaagtttcattttgtttttggtgaaggtcacaaacctccttcttccttgactcagagaggaaaggtcccagtatatatatttgaatggtcagtggttttcaagaccttcagagagctccctgcattttacctagaaacagaaaaggcctgcaaaatcttaagtttcctggcctgcattttccaggtaggggcaaatgactccaagctggtctctaagccaatacccttgtaaaccagagtccaggaaagatagctcaagtgtgtaattctctggagctcaattctatgcagttgtgctatttcattaagtcactgtgtgtttttaagtgttgatacattaaaagtcgctttatggaagatgagtaaattttttaaatacttggaaattttatttccttattaacttctacagatcagggcatgcaaccaaaagcagcttaaatgaaatatttaaaaaaaataaaatatcaggaagctatttttagatttcttctggcttatgtttctattttaggaccctcatttttctcttattaaaaaaaattatttcctgtacatctcatggactgcagggtaaattatttgggcataaataatttaaatagttttctttcattttgactatctccagtaacaacaatttttattagccagcatattggcttattgcacaaatcttaaaatgtacattgactactttttgagaagaaagtggtatcagtactcgcgatgaaaaggttactactgaacaaattcacatttcaggaacacctatctatctttggtttaaatcttactcttagtttttcagtctaaaaatcatactggtattagtatcaggtaaggaaattaaaagttttttaaatggtttcattctctgcaatatgcaaaatttagattttactttctggtactgtaaagaacctgaagtgattcacacttaatgggtcattaatccagtattctttaccctgaatgtttggatattaaagttcctttatgttttctataa

SEQ ID NO: 3 - DNA, 29187 bp, Mus musculus
ggagggtgagcatgctagctggctgtgcgcatgcgcggcacgggtcactcggctgccggctcctaggcgtccggctcgccgccatcttggatcggggtttcattgttcgcggtaggccgggcctttttttgtgtaatcgctgccaaagaagaaagcgaagtagcctccggtcaagcgaggaggccggctcttccgcagccatagcccgcctcaaggatttgggaggtagcgaagggcagggagctggaccctgaggtgccgccgcgaccgcagcagccatggaggacgagatgcccaagactctgtgagtctggagcagcgttgagggaggcgggatggtggtcgccccgtagggaggccgcggccctgcgcctctctgcagcctattgttctccgtggacgccgcgctgtggtggttcctccctcccgtgcgcacgccaggatgcttagagccgagccgggccgagaggacgatggcgggagaggggctcggcccctgtcacccctcggctggctgcacccgcggcccccagacctggaggaaggacttttcattcaaagctctcctcaggactcggcacgtcctcctggagtcccatgcttgcgtcctttttttccccgccccctcttttcgatgaatcttttccagatttccatctggaacgtcctttctcttttcttttggtaggatttttttcatttttaattcttgaaggctgaggttaaaaactttatacttgcagttgagaactggaaagaaaaaaaaaaaaaaagccagacatctttctcgaaggtcttcggctttccctctgtagtactcccctgaccctcgaccaaaaaacctaactggacacccaaggatgttgaaaccgttcttaaaaaatggaaatctcactcggagggatgcatctaccgagggaaagtgggtttcattaaaacatatttaccaccatttgcgactagtggttgacatttttattaatggagtaactgtggtcagtcaggtgtaagcatctttagtgaatattttgtaactcgagtctaggatattgtctttgtcatgtaacttccccttgcattctttgtttttcaacgcacgggttgaggcggttgtgcctttgttgggtttcacagggcaaaaaagaaaagctcaagaaattgtgggttttggttgttgtttttttctcccggtatatttgaagattgcaacttgatgtatcgtgcatatagttcatataaatgaagtaggatgaacatgtatcatgttacgggccttaactctgcacctagttcctgccatagctttttatttctagaatcctggtacttttagagctatgtacatttagggttctttttgacattttctagttattaaaattgttctgaagtacagatacgttattctttttcccctcgcccattttcaatttcttttttcttttgtttgttttgttttattgagacagggtgtatctctggctgtcccagactcactattacagaccaggttggcctaaaattcagagatttgtctgcctctgccttctgaacactgtgattaaagtctgtgccatacactttgccccaatttaaatttcaagttgctatttttaatgtattatgtgaaatatacttactttttttttttttttttttttggaaacagggtcttttatataacccaggttatccttgaactatacctatactggctcagaactgttagttctgtctgagtcccaggtgctaggattcagatattgcctacccagtttgcatttttgtttcttctccctccctccccatcctggctggcctggatcttcctttgtatagtaggcaggcttgcaaacttgtgaaggttctctttcctctgtttcccaagtgctgggattacagacttgtcccaccaaaccacatttacactcttcttgatatattaaatatcttgttagtttatgggacctggaagtagctttttagtaaggatgcaatacttagggcatggccatttttttttttttttggaaagattatatctataggtaaatactccctaaaacctattaaatacatatttattggaaattttccctttttcatcttagcattttagaattcatgctgctatgctttttggtgtctttataagatacttgaacactttttaaagcttcattcctgttgtaagggtattttgcctgcatgtgtatatctgccttcagaagtcagaagacattggatctcctggaactggagtcaggtgtttgtgagccatgtgggtgctgggcaccaaaccaaggccctcttcaagaacaggtgctattaactgctgagccatctccaaccacactatgaaaatcttatattttttttattttaaaaatttgcagaaatatatggatgataacattaccattttagtatgtacatggaaatatgcagtaaggacatgttatcatttccaaagcaggcatttaaatagcactatattgagaccaggaaagtattgattggagtagattcatccctgggaagttggataaatccttagtgtgtgtcagatgcttaccatgctcgcttcgtctttattatcactaaggggaaaggcattttataaccaaaggaaagctaagcttaatgtcccaacaactggctcaaaagcatatactttaattaaaaaaattcttgtctcactcgtctttcatttcagcaacttgtaaggcagaggcagacggatttctgagttagaggccagtttctggtacagagtgaattccaggacagccatggaaaaagagaaagagacccaaaacaaccctgcaattttgttacctgttacagcagtgcctactttttcttgatgtccttcagtaaagcagcctattgaagagtttccttctgaaagtgtccgtgatttaaagttccccattatcccttccctggtgctccaccccttctgtcaaaacctccatccttccactcagccagtactgattaaatcttggtaggtactaactctaaaaatgttcccaatagtgtttttaagttttaagtgctcacattagtgatcctggaagccagggttttctgatttaatgtgtattcttagccacagtctagtccatgggaagtaatcacacttgttttaaaaatccggtacagttgaagaagtcaagctgttcctttgggagtagcctggttacctcacctgtgttcctagtgactccgaggcagctgcttcctaagcttagttttcagccacaacaccttgttaagttagagattgtcagcaatacttttaaaatatattgtaagaaaaatcaaatgattattcagcaagataattttcaggaatattcaaagtatagtttagagatttatagtgagtggttgggttatgtacacattttggtttttttttttttcgagacagggtttctctgtatagccctggctgtcctggaactcactctgtagaccaggctggcctcgaactcagaaatccacctgcctctgcctcccgagtgctgggattaaaggcgtgcgccaccacgcccagctatgtacacattttattcttcccatgtgtaagttttaatttgtccataataaaaaaattaaaaatatgtatagttaagtctgagttggctttggtcactttaaaactttgaatttgtaaactagaaactgtacaccaaagtagaaaaacatagtgttagtgttagctgatggatttccttggatccacctgaacatcttttgtgtaatttactgtcaaatgcagatggtacttttgagacagctatgtagttattgtttggtctttacagctgctaggatcgtaggtctatacattttatagcatcactaacagcctgcagctaagtaattgctgaacgctgatctggactttctctttctacccccctgtgtagttgtaatttttatccacgcccacttctctgcatacagtttgacgatataatgtctatgaaatagcatctctttttctcttttgggcttagttgtattttcttgtgtttttccacttcttttttattcttttttttttttttcctctcgagactgtgccttactatatagcctagcttggccttgtgcttgtttattctttttttttttttttaaggattatttatttgggctggagagatggctcagcagttaagagcactgactgctcttccagaggtcctgagttctattcccagcaaccacttggtggctcacaaccacttgtaatgagatctgacgccctcttctggtgcatctgaagacagctacagtgtacttatgtataataaacaaataaacaaatcttttttaaaagatctccattaaaaagtatttattatatgtaagtacactgtagctgtcttcagactctccagaagaggggatcagatctcattacagatggttgtaagccatcatgtggttgctgggaattgaactcagaacctctggaagagcagtcagtgctgttaaccactgagccatctctctaaccctacagtttctttccttttgagacaaaatctcacttttgtttgaaaggttgcctcaaacttgtgatggtcgtgcctagcctccaaaacgttgggagtaacattttatttggggtgggagatggaagtcaaaggacatccttttgagcaggttctcccttttatacgtgagttccagtgattgaactcagatgtttggcagcaagcaccgttacctactgagccatcttgccagccctcaccctcacggttttttattatttttgttttgttttgttttgtttttcaagacaagatcttactttgtagcccttgctgtcctgaaactccctgtgtagaccaggctagcttctcctgtctctgcctcccagattaaaggcttgtgccactctggtttctttttcttatcttcttttttggtttttcaagacagggctgtccctggactcactttgtggggcaggctaacctccaactgcctctgcctccgaaatgctgggattaaaagggttttgttttatttttaagatgtcttttacttatatatatatatgcatatgtatgtatacacacacacacacacgcatatatatatatatacatatacatacatacatacacatacatgtatatatgagtacactgtagctgtctttacacaccagaagagggcattggatcccatttggttgtgagccaccatgtagtttcagggaattgaactcaggacctctggaaaagcagtcagtgctcataaccactgatcaatctctttggatgtttctagttcttgaaagaaaactacaagttttctcataaagtttaggaatcaggcaaaataaaaaattctctagcaaactgcaagtgttcaagcatcattctaaggattaaatgtatcagaccctgtttttcttcctcagcagtgcataaataaccaaccaaggtttaaggttcaaggcctgctttggctataagagaccttgtcttaaaacaattatacagaatttacaaagaactaataaaatggaaaacacaagtcttggagagggaatcattgatctcttttaaccctttttccacttgcttactctctgtatcaatgttttgcctgagtgcacatactcacacacaagcaggcctgcacagttcagaagaggacattgcgtccctggcagctagagcctgtcagagagttgtaagctaccatgtgagttctgagaattgaacctgtcttctgtaagagcaacaagtgcttttgacttctgagccatctctagcccctcattagggcttgatatattctagtcagggttcttctagaccagtggttctcaaccttcctaatgctgctacccttgaaatgagttcttcattttatggtgacctccaaccataaaattattttctttgctactatataactgattttgttactgttatgaatcataaatatctgatgctcaggatatttggtattgtatccctgagaaagggttgttcagccagcaacgagtcatgaaccacaggctgagaaccaaagttttagactgtagttacctaacccatttttcaaaatctctgcttaaaaagaatttatcattagtttaaaatctacttaatgcttttccctcaaatttccaggttttccattataaaataaaatattttaatataatgtaccaaaagacaaattcagtttaatgttctgctttgtaaattcttttgtaaattaactatctagagctgtataaataaaaatgaatatttcaagagttttggattccattttcataacttcgattttagacgggagtggggaatgttgagacagggtcttctagagcagttgtcgtccctaaactctgatctttctacctctgcctcccaaatgatgggtttacagaagtgttcctacaccatattgatttttacaactacctgaaaacttagctttccacttcaaaattccatatggctcaggggataaagttgcttgcctagcacatcctagcttcagttgtggcacattgagttgactgaactctgtcctgacagagctggcctgttgtcactagtagtgttagtgcccttacagtcaaatctagcttgttagtcatttagaaaagctacagaaaaattacaggaaaggtatactgaaaactagctgcctttcctgtggaattaccagttagttcccatttagccagctgtgcattatttatgtttccatgtgggtttttggtcggttggttggtttttacatttttagattgcattcatttatgtatttggtgtgtgatgtgttggggggggtgtcaaagcaaactgggttggatttgttcttcatcatgtgggttccaggaatcaaactcgggtggtcagactttatgtcaagtgccataactcactgagccatcttgctagctcaggtttggttttttgaatctctctctctctctctctctctctctctctctttctctctctgtgtgtgtgtgtgtgtgtttcagtctttttctgtctccttccttccatctttcctttttcactaaagtgtcttagttcaaaatggtgacacttcaatctcaaatattgtataagaacaagaacattctcttaggtaaccataatatgctttcatcttgaggttatattcatacactgatgtgtatgcatatgcatgttgtatgtattggtaacactcaaaagcaggggttctagtagtgcctttcccagcttcccagcttggttagatcttcagctccagatacagtgttacctaatatattcgtttacccaagtgtgtgtgtgtgtgtgtgtgtgtgtgtgtgtgtgtgtgtgtgtgtgtgtgtgtgaacccttgtatgattgtgttaggagattgtagaccctttaagaggtgagtccttgtgccgggtgtggtggcgcacacctttaatcccagccagcacttgggaggcagaggcaggtggatttctgagttcgaggccagcctggtctacaaagtgagttccaggacagccagggatacagagaaaccctgtctccaaaaacaaaaacaaacaaaacaaaaaaaagaggtgagtcctgtgtgtgtgtgtgtgtgtgtgtgtgtgtgtgtgtgtgtgtaactccagcattttagaggtggaagtaggaagattaggagttccagactagtctgaactatctagcaaaacctcgtttcaaaaaaatcaaaacaaaagaagtgagtcctcgttaaaggctgtaaagtcataagagcactatcttgggaaaagattagtagacttagttgttaccagcaaaaccaacccaggttcaacatgtgatacagtctcaaaaatgctagggtggtgtttaaacttccaccacttggaagctgaggcagacagatctcctgtgagtttgaggacagcttgagttccaggacagctagggctatatagagagaccctgtctaacacacacacacacacacacagctttctgcaggaagagtgtggaactttgcttagaaaatattttggaaagagacaacttttagcccacattcagaatcaaatgatagtctgcctcatcccatccctgaaaagatagtactccacaccttcccagggtcaggcacctcagcactgctcctgttgttgcttatatgctgccagctgtgttttcttcaggctttgtttcttttgtttttccatttcagggtctcactgagtagcccagcctgcccttttctcacggttctcttgcctcagcctcattagtgctaggattgcagatctgtgctgttaaacctggcttaaggaaacagttctaagtaaaaataagacaagatatagactataaattctttcattaaaggccagatatgatagggcatgcctgtaagctagcacacagaagactgaagcagaattaatggcagcttgagaacaccatggtccacattatcataatcctatcttaaggaaaaaaaaacatagaaaagaaagccaaacattttaaagtacaaagatgttcttacttttttgctcctgcacaggaaggatctgcagtttgcacatttgttcgtgctttctcttcagaactagtatttgttcagctaatagacttgacacagcaaacagcattgtttataaaaatttgagggcaactttagcaactaaatttattgggagaattggttcagtccctatgctgtttgcataaggaagacttgtcctgtgatttctttgctctccctgtccccacatttgagacagtgttttcttatgtgttccatactggctttgaacacctctgcctcacaagggctaagattacaggcatgtgccaatacatctgactgccttcctcctcctctcccttactctttttttcttcctctcttcctccctctctgaagcaagattttgcatatcccagactgacctcaaattgtgtagacatgaatttcagatcttttggcaaagagggggccccttggtccctaaggcttagcattgcagctctcctgatctcctgctcaaaaggcttggaaagtgcttcgctgtcctcttgtaggtctcatttttggagtagtcagctccagcatgaccaaattgccattagatatatggtctccttccaagtccatcttttctacccactccttattctacccactccttagtgttacttctcagtgcttctccctggccttcttcctccagcatcctcatggtctcagcggctctgtcctgaacactaggaggaaagaaaccatttttacattttgtggacatgcagaggtaccttttatcttgaaagagtcaaagatatatctttggctgctcccaacatttatttttcaaaaagcaaaattcaacaaaagatttttcactcctgggacaggagctcactctgtaaccataactggcttagaattcagcatgtggactagactggccttgaacccgtgcatgttgggattacaggtctgaggcaccatgcttagccataaataaatctaaaatggtaaattgtttgtttccctctgttccagatacgtcggtaacctttctcgagatgtgacagaagctctcatcctccagctctttagccagattggaccttgtaaaaactgcaaaatgattatggatgtaagggtatttcactcagtcactgttcttccttccttccttccttaagattactaatgaaagctgtcattactgcagaacttcttcattaatgtctagattcatgaggatcttagtgaatttgtaattaacctgtttagtaggcaaccaagtttcttttgctaagtgaatggtgtgtgtgtctgtatgcactcttctaggttctattgaggcatcagaattgttttagtgttaattggctgaatgatctataagttcagttattagattgggaattggggtagacttataatgaaaaaatatataatgaaatacatttttagcttcatttttaaaggttttcttgaatgcctttcagactttaatgtgtagaaaacttattcatttggttggtgttaactacaaataatgagcttttaaaacaaagttaattgtacacgtgagcaggtgaatggatatgtatgtggaggccagacgtcaacattggtggttccttgtgtatacatagtagacagggtcttacactgtaatccaagatgacttggaacttgctatgtaagctatagtcttcctgacttagcttctcaagtgctgataggccaccacagcctggaaaatatctgtttgtttatttatttgttttttgagggatggtctatgtaattaattcagactgccctgaaacttggactgttctgctttttgagtaaaagaatgataaatgtgctatcgccccgcagaatctcaattattctagaatacaagagtgtttactgaagtttatgcttatctctatgttctgaattttgaattagtttcctttaataattcttaaaaagtcttagaatgaaatacatgcattataaagtcatttattattctgcacttttaaatccagattattagatgtattttttgctttaatttatcataaaatatcttgaagccaggtgtcatagtgcgtgcctataatctcagaactcagtaaacagggaacagaggatcactgcaggttgggggccactctgactaccatagtaagttctagactgcccaaggcgcaatagaaaaactgtcaataaatacataaacaaacaacattttgctttacgctgaaggttttctcagtgattgcttccttagcatgcacaaggccctggattttgggccccaacactaaggggagaaagtccaggttttgtttattgtatcccagtcaccagcagaaaagaagggtttgtttttttgttttttgtttttgtttgtttgtttgtttgtttttgtcttttgagagatcttactaagtatccagactggctttagacttcaccaagatgtcagcatccctgatcctgggattacagatagagaggttttttttggtttggtttggtttggcttttcgagacagggtttctctgtgtagccctggctgtcctggaactcactctttagaccaggctggccttgaactcagaaatccacctgcctctgcctcccaagtgctgggattaaaggcgtgtgccaccatgcctggctggatagagatttttttctctcagatttaaatcttttattttgtatgagtgttttcatgcctcctgcccaaggaagtgagaatatggtgttagatcccttacattggagtttatggatggttctgagccactgtataagtgcaggggaacaaacattggtcctctgcaagagcaacaagtacttttaacctctgagccaactctctagctcccaaaggaggaaattttaaaattcttctcaataggcaaagaatctgtaccagaagttcatattctaaaacatattgtaaatattatcagaacctgttagtgtccagagtctagtttagtctggaaaagattagttaagattttctattgttccacaaattagaagcagtaagatgtcttaagtttgtaataattaaaaagtaatatagagggtggaagtacaccttaaggaagagcgttgatacagtggctctaaggctctgggtctgacatagcactgaaataatcatacttttattatatgaaataatcatacttggtatttccttgtttttagtttttagtttttggtttgtgtgttttttgagacagggtttctctgtgtagccttggctgtcctggaactcactatgtagaccagactggccttaaactcaaagatctgcctgcctccgtagtgctgggattaaaggcctgtgctgccatcacctggttctggtatttatttatttttatctgaaaatgtacgtatggaatgagtaggtcattctccatgccttgctaactgtagaagtttatgtgacaaaggggacatttttatttcaaatgggagagtaaagcccaaaactgacttgtgaatttcttgttcaatagacagcaggaaatgacccatactgttttgtggagttccatgagcaccgtcatgcagctgcagccctggctgccatgaatgggcggaagataatgggtaaggtaagctgtcagagttagagcaggtgacttgcgctggagaacagttttcttcatcatcttcctacttctgcctcccaggtggaactactagtgtatgccaccacaccccaccttatgcagtgtgtggctttgaacagcactctacaacaaagctacatacccaggtcttggaatagaaagttttaaattggattttaccgtgtttctgttttagaaccattatatttcctttgtagcaaagggtcactttgtatttgtattaaaaaggtaaaatttggctggagagatggcttggcgttaagaacattggctattccagaggacctgggtttgagccttgccacctgcatggtagctcacaattctctgtaccactggtcataggggctgtaatgttttcttcttgcctttgaggatactgcacacacatgatgcacagatgtacatgtggcaaagcacccatacataaataaatagaaactgtaaaagaggagctcagcacagtggcacatacagtgttcttgattctttgtaaacttcatatcatgtgccctaatcctactcaccgccccacccctccacatgccttctacccttggaaccttacccccaaaagaaaacaaaacatcaaaaaacattaagagaaaagaaatctcactatggaagctatggtacatctcacggtgtcgtggaagctgtgctatgtcacacagtgtcacccttttgcccacacagctttacttgcaaatgttcgttacagtgagtcattggtctggtctgaggcctctggcttctgctacactatcaatactggatcctcactaggtaggactcctctaggatatcctgttttagagatcctacaactgtggtcctacaggcccagccccttcctgtcctccagcagttcatagatgggatagatgttggaatggaccaactcaaggccctggtctgagcctagtggtggctgtgttgttcagcctgccaactctcctttaccaggaccaccagggccagctcttcagcttggcctggatgaggggcaaggccaaatctcctgcttatagccaaaagtcaggactgactcacatgttcaggactggctctcagggccagcttgcccttcatggccaggtgaggtatggggcctagccttttaatcaaaacagtcaggaagcagaagaaggcagatttctatgagttcaaggccagccaacactacagagtctagaatgaatgagagaaggaaagaaagaaacaaattaggctaaagcctctagaaactggagacctatgttacctccttttccccatggtgctgtgcacatgctaactaccagatagttatggaactactcaaatttgaatgaaacataacagtaatatggaaaactaatggagaggaacccaggggaaatgagtggataactttaaattgtggctcggagtggtggtgcatgcctttaatccagcactagggaagcagaggcaggtggatctcttgagtttgaggccagcctggtctatagagggaattataggactgcacagagaaaccctgtctcaaaaacaacaaaaaacgaaaccaaacagaaaaacaaacaagtggtgcacacctttagtcaaagattggagaggcagaggcaggtaggtctcagtgagttggaagccagcttggtccacacagtgagtttcacgacagtaagagctacataatgagaccatgtctcaaaaaaataaaaagacaaaacagaactaaaaggttaaaaaaagttatttgtacattagcagaaatatgcagagattaggatttgtcaaatatacctaaattagctggacctacattcttaggcaagagaattgctagttcaaggcttgcctaggctatagagtgaattgacacttagcctggctgagttggtgacttatcttagagtaatatgcagagaggagcagagccaaaaatgctgctcagtagtagactgcttgcttgtatactatttgtgaggctttaggtttaatccccagacctagcctactccccccataaaaagaaaggatgaaaagcatgttggctatatggagagctcagtgatgaagaacacccttgatgctaatccagagggtctagatgtgattcccagcacccacgtgatagctcacaatcatctataacttcagttcctggggatccattgccctctttacttctctacaggtgtgtatatctggtagtcggtacaaatgtgcatagacataaattaaggcaaaccacctatacacataaataaaatcttaaaaaagaacagaaagatctgtacttacaagttataatttgtcacactaggccagaagaagaatgttgaaactgtgatgagcacaggtgtcccctgcaccatctctctctgctgtgtcgcaggaaaacgtgattttgagctttatctttatggaatatatatatggttttttttaggaagtgaaagtgaattgggcaacaacccctagcagtcaaaagaaggatacgagcagtaagtacatttgatgccctgtgggcgtttgttgtcttataccccatagttctgttgtaaagcatataacatcatacatgtttgcagtaatattttgatcgttatcctgatatgattgaatgtgtttgtgttaataaatttaagaacaaatctaatctttgtcatcaggtagtaccgttgtcagcacacagcgttcacaaggtaattgtatcttcttaaacataaaaatgaactcttgaaagggtattcactaaccacctcaagttttttaatttgatattggttggggggagcggggcggaaatatatagtatttttatttgtttctggcaatattttttccttcttcccattgttgaccaagtgtggcttgtccacgactttgttgctagttcaagctctctatcccccttggtagcagctgatgccttagaaatactttcaccacctaaagggcaagatcccctacttccctgaggtcacctgggcttcatacccagatcttccagtgctcaagttgctcctgaaacagtgtttgcggaagagcaacccagggtgttgcttaaggcctgcaaataggggtaacttctctttccaaatctactttgtcaaagttgatgtctgtgttcattgtaaagtgttcctcagcaaggttggctttccaaagcttcacgtatgggatggttattttctctccaatgctttcttaatggtgccgatataaagtgaagggattactgttttccttctgttgccttcagtctgagttcacttgcacatggattcacataaactgaatggtgtaatgtctgggcaaccaaaactgttggcttttgagaaaactgtcaaatactttaacatcaaactgttgcaatgcaaggtatttctttgattgttcttcacaaaataatggctaaaccaagtatttcatgtagctagcttcagtaagttgccttaaataaggcaaatctagtcttcataactctcagtacaactagttaattgtaatttataaagcctagatgtagtaataaaatgagtaacattaaataattactgttagaataactaagttttactttttttttttacagaactgaaagtttttatgttagtagtttgtttcctagacccttataattattgtaataactgtaaatagacagtgactaccagcttctggtagctatcagtccttttagaagactactgtgttgttaactttgatttgttatgtttaagattgataggattttatagcgaggatagtattttatttatacctaattatatagtctgtgcattattttaagaatactttattgggtcagttgtcgtatttaagttctgaagttgtatttttcttttcactcccctccttcctttgtaagatcatttccatgtgtttgttggtgacctcagtccagaaatcacaaccgaagacatcaaagcagcgtttgcaccatttggaagaatttcgtaagtagcaaaagataaatataacacttaattagcagtgattttttttgtttatgtaataataactataactttgttatgactgtgaaccttaattttaacactttaaatatagcatattccattgtttagagatcatccaagagtctgaaagcaagtacagtcccactaatgagtaatggtcttcagtaaccttaggccctgctcagtcagaaacgcttcctcagactgctgtcacacacttcagcctcctggaggcgctcagtcagaaacgcttcctcagactgctgtcacacacttcagcctcctggaggcgctgtgctgtgattgaaggacttattcaggcgcttgtatgattccatgcagtttatgttaatttaacaacacaaaacctgaacagtttagattgaaaaaaacagatgttaaatattccttccttggcatttaacccctcttcccttttaaaaacgtaacagtagtgttttgtactatattataaaatactgtcgccattaaatttgctagtatccatgcactttaggccctttgtcagtatttattcagggtggtagttaccataggagagctttatcacagtaagagtaataagccatttcagtactgactcaggatcagttgctgagtgtagggaaagtattgaagggtttctaattcctttgttattgtgtcactagaacaaaaaccaaaaggaattctatctctagctggtttgaacagagcagcaggtaaaggtgaggggattgagaagtagacaccctgagcattgtgtgagttttcagtggagagttgcagttggctggtgggagaactactgtgagggtagggcagcttacagatggactctgctgttacatgacattttcctttgagagaactggctaagaaaatagtgaggctttaatttcttgattgttttttataacctgcgttaggatttcactagtggttggtttctttcaagtatctagactaggcacgtctgactaactttgcctctcaagctctgagggttccgctttgcagaatcttgtgtgttttcagtttggtgtagagaatcttggtcagtttcatcctgtcttcagtgacagaagacacaagttctgatctgtgtgctgctttctgtgtgagtcattttagaaagaagtaagcatcgactgatacctcagggcagacacttccaattccaaggcattggaaagaaaacaactccttctgatattgtgaaccaataaagtaactactctggtggatttttaatttttattgaattaaccttttctaatagacttttagattaagatttagataaggagtgtttaatagtgattgaagggctactagagtggttcagcagataaggtgctgctaccaaaccagatgacctgagttcagtccccaaattgcatgtggtaggagagaattaactcctaaaaattgtcctctggggctggagagatggctcagcggttaagagcactgattgctcttccagagattctgagttcaaatcccagcaaccacatggtggctcacaaccatctgtaatgggatctgatgccctctgctgggtgtctgaaaacagctacagtgtactcctttacataaataaatcttttataaaaaaaaaaaaaagaaagaaaaaaaattgtcctctgacctacacacacacacacactaaatgttttataaaaaaaaaaaaaaaaaaaagccagaggtggtggcatacatctttaatcccatcacttaggaggcagaggcagatggatctcttgagtttgaggccagcctgatctacagagtgagttccagggtagccagggctccacagaataaattctgtctcaataaaataaaataaaaataaagaatgtaattccttagcaaatagtcttagaaagggcctccgtcagtaaggttagaacagaagggcttagaaagctcacaggggcaggggctcacacctgtgaacccagcaggtaagagaaataggtaaaatcaaatgacattcagacttagcagttccatgtcttttagttataaactagtcttcttgttactataactacatcatattttagtctagtttcttttacatgtttgtgtatgtgttttttatatgtgtgagtgtatgtgtgctcctcttagacaagagattgatatcaaatattttcatccgttactttccagctaataaactgaggcagagttctcactagaacctagagcttaccagttctactagtcttgccggcttaccctgagtaccccctgcctctgccccccatgcatacttactgtcccggcatttatgtgggtgctggagatctgaacttaggtcctttcactttgcatgatagcacgctctctgctgagcgtgtccctagccccagcatttgggttttcttataggttcacagttgttagcatattaattcttttcttgataaaaccttttcgttgagttaatatgtgtttgaatgttagcttatattggtagttagctttcctaaatgtaaaagaactatttggcaattggtaacacataacatgtttctatttttgtttgtgtggttgtttcttgtcttaaggagacaggctgatagcagtgttgaaaggtttctacagtagatgtctctataggtggtttacaccatgcttaatttatagaaggtgtggggtaaatgtgatctattaaatatttaatatgaaaataaaaatattttccctaattgatatagcttaaaccctttctgtttcttgagttcagggtcatatagaaatttttgatacaggaataaatcaactaccaaattaaatgcatgtgttccatgtaggtaatagttactgagaagtgacttatagtagctttacctaatcatcagaatctttttgattacacttaataaatcacttctaaactgatctaaaatatgcttgtgtatacttacaattaccttgcatggctttctgacgtgggttcgtgttgtaaacatgcctgttttgttcatatgtcttgagagaacaagcattgtgacatatactaggttgatttaaagtagattgcctgtgaaacacagtttgagtccagtttttttttttgaggtatggagttttagctatcgtggctgggtttttactcaatctcaaataatagggctctggttattttgcagagtgtctctgaagaatggacagaattgccctggctaactacaagctacggttcacagtggataaatgttggcgtgcttttttactttctgactttttaaaactttgtttcatatcttgtagtttccaatcaatcttaatataaatgctgttaagaaattcagtataaaatcttagtttaaaagtaaaatgtgtttgtgtatccttcgatgatttagcatttctaaaaatgtcaagttctcaaaatttaccctagcccagaatgtgggtgtatattcatctgagaaaatgaagtaattagcctagggaatatatacagctttttaatcctttagttttcaattttgtattttaatttttttaaatgactatgtaacatatggtgccaattagtaaataaattgcatggttaaagaaagccaaagtagctaagaactttgtaattgctaagtgcttcataggaacatctgctttcaccatgcttaatattgagatgttcataaaacattttgaaatggaaaactctctttatactatagatttatatttaagacattgatttgcatgtctataaaattaaccctcattagccaataaatacgtttattagaataaaaggtgaacttttattaacggtttactgttagtgaaggtgaaatgtaaaagtgaattgtatttggctctcattttatgcctagaggatggtttacaaaactcaatttctggtggcttctgtttcagagatgcccgtgtggtaaaagacatggctaccgggaagtctaagggatatggctttgtctcctttttcaacaaatgggtgagcacaatcataagtgcatatccaggaccatagaggagggtgaaggaaagcgtcagcttgttatctgctagtgttcagttcactaagagtaagcaccttcactagaaaacagcctgttctattctggttggttctttagtcatttagcaccaacacatgcaaaatgctgatactgtatttcaaactgtgcaaagtaaaaatgcttatcaccaaaaagaaaaaaggaaaaaaaaagcccccaaaatgttttgtcaagatatctgaccatttgtaattgctcagttgtaccccaaactgtgaggtatatatactgctgcacctacgtatccttaccctttgtagcattctccaaacctattatccaggaaatgaaatttaaaagcaatgttgcaattttggaaagctactaggtattgtgtgttccctaaggactttttcattttttaacatcttcaaaaagcatcatgttttctttatgtaagctgtttgtaatgttaactaatcggagcttttttgttgtctctgatatgtggaacaggatgcagaaaatgccattcagcagatgggtggccagtggcttggtggaagacaaatcagaactaactgggcaacccgaaagcctccagctccaaagagtacatatgagtgtaggtgcattggagaagagaagcaaaggtggcattgtggagagaaaatacactagattgtaaatgttagcgctgtccccggagacttattgcagaaatagatgagaagcagatcaagactactgttcaaaatgtacttagttttcattcttgtgattttaagtaattatctttaatgtcaagtctcctgttaagtagagcatatatatggggtaatgtttatgcattttgtggtggtggcttaatcttaaaataaaatgtaatgtgttttataggtattttgattgagaaaaactgccattgtagtagtgtagttaatgagttaatccttccacatatgggtgatgatttgaagatagaaaatgttctctctcctcagcgaacaccaaacagctgtcatatgatgaggttgtgagtcagtctagtcccaacaactgcactgtgtactgtggaggggtgacgtcaggactgacaggtatgggagtttgcctgggtggcaccttgaaactagcttaaactgaaaagcttcatatttataggttttaatagtgcgaattctttttatgttctttgtttttgttttctaacagaacaactaatgcgtcagactttttctccatttggacaaataatggaaattcgagtcttcccagataaaggatactcatttgttcggtaagggtagtctcttttaaagctttttttcttcagaaataattttgaacatttaatatagtggttttcttaagaatttgtcttgatcacttgagaccccagctcaggtgtctatacctggtaatactaaaatcttgagagagagagaaatgtctttagcagatagccatgtactcatttttactattcacttttttatagacagcatctcactgctgcccaggctggccttgaactcacagagctccacttgcctctgccttgaactcacagagctccacttgcctctgccttttaaactcacagagctccacctgcctctgccttccaagtgctgtatttacaagctacttactggtacatttttataagtccctctttccaactcaacaccaagaaaacatacgcattgttaaaacaaatttcccatgccaggtgtggcggtacggatctgtagtcacagtacttagaatgagtacttaggttcatggtcacccttggcttcattagagaattcagcttggcctacatgagaccctgtctttttatttaaaaaaaaaaaaaagaagaagaagaaagaaagaaaaatctccaatctccacagctgtaaggtctttgaaaaaaaatatagcaaaggaaatatgccacttttggggggggggtaggttgggattatttcagaatcctctatttcttccatgtttgctttgcttttttttctcccaaaatggtccagtttatttttcttgattttaaaaattgtgttcataaatacttaaaagagaatttatattgaaaattcttaatattgtttccaataggttcagttcccatgaaagtgcagcacatgcgattgtttctgttaatggtactaccattgaagggcatgtggtgaaatgttattggggcaaagagactcttgatatgataaaccctgtgcaacaggtgagagggtccttgactgtgggatggaattactgggctagtattgagcttgctttaaaatgatgcttattgctaatccttgttaacaagtttctagctcaacttcaacttctataagggttcttcctatgtgtattatagttccgagtggtgcagtcatggattgcagaggtgacatgatgagtgtaagcacttcagttgactcatgttcacaaagtggatttttaaaatgctcggccaaacctaacagggtattaaatcttttcttaatacttagtaaatatgtccacattgatatggaaaacccgtaagcatgcttaacccgactgaacaaaacataaagctataaatatatgtaggcattagtattgttgctttgttttctaagcaactttaatggtccaagctcagccccagagtcttctatgagggcattgtaaatggagcataaagataccaatgagaaaaccctaaacttacatttccttttcttttttaatgtttatttatttattatatgtaagtacactgtagctgccttcagacacaccagaagagggcatcagatctcattacagatggttgtgagccaccatgtggttgctgggatttgaactcagtgctcttaacctctgagccatctctggagcccctgagctcttacatttccaatccaaaggaaacaatcataaacagttatgaacagcaagtccaatctttatcatgagtttttgtctgagcctttcaaagactgcaccacatagttacagtagcatatgtacatacataatggcacttttttggtggtctttctctgtgtgatgctgggcactcgttctagcactgagctgtgttccccagtcctatcctaatatttcagttgtggtaatggccctatgctttgactgtgtggtgtattaagactgtatataaaaagaacttcaagtaatactcaattatctgttgtaattttaattgatagcaaaatcaaattggatatccaccaacatatggccagtggggccagtggtatggaaatgcacaacagattggccagtatgtgcctaacggttggcaagtacctgcctatggagtgtatggccagccatggagccagcaggggttcaagtaagcatattcgcgctttgcctttcttaaccgtggaaaacagtaaaaggaaacttgcaggtattgagtaaaccatgtgacctcagcgttaagtgtggtgatagccttgaaggtttacccctggcctccatatcttctattgtgcttgtacatctttgccccagtacacgcacacacacatgcaaacaaaataagatatttttcctagaaatattttgccttgaaatgtaatcacaacactatatattttggtaaatgtttaataatgaattaacatttttaaattatatcttatttatttgtgtggcacatacaccactacatattcatagtgaccaggggaaaacttgggagttctctccttcactgtatgagtcgtggggatcaagcttaggttgttaggcttagtggcagccttgacccagtgaaccattttacccggtccctatttttgttttctgaggcagagaagcccatgctggctttgaacttgatataaaactggggatgaccttgaactcttaatcctcctgcttctacttcccaattataggggttcgataccacatcgaacttaaatcaactttctagtgaaaatagctgcttatcttgaatacacaagtgtttaaaaggtgtggttctttgtctgtgagtttcttgcctgcatatatgtttaccgccctggtcccttgcagtgatcaggagagggcatcagatcccatggatctggagttagaggtagtcgtaagccaccatgtggttactgagaaatgagcctggtcttctagagcaacaaatgcccctagcccctgaaccagttcttgcagcccctgaatatacattgttcacccatggagaaagactctccctccatgtctgtgagttgtggcagtccagagatgcccctttctctctgggtgtctgctcttctggaatgaaggctgctctaggagctttcctctccgctcccctgtgccgtgtagtctattgcataagctcagtctgtgtgcaatgttatgtctgtctgcccactgaccaggttccagtttatgaaaggcatttgagcagcaagattgagtcctcattctcaaagtaaccagtgtctcacaggtaacatttgtcagaactgatcacgtgatgatatagaactctagcttttccctcctgtgtttctggtgtttgccagtgcagccctgaccatgctcacatgtccctgcgtgttcttcaggtcttccttgtgaacagatgaaataaaggatcaaaacaagtatttagatgcattcattgtgcatatataactgtatatgtacatatacatatttatatgtacttggaagtacttggaaggcacaggccagagcatctctaatttggaggccaacctgatctacatttcaggctcaggatacgtggaccttttctttaaagagagagagagagagagagagagagagagagagagagagagagagagagagagagagagagagaagaaagaaagaaaaacattgtgtgtcagattaaagatatttttttaatttattttttttgttagatattttcttcatttacatttcaaatgctatcctgaaagtcccctataccctccccccgccctaaagatattttttaaagagctgagaggatggttaagtagttaagatcacttgctgttcctgcagaggaccaggctacagttctcaacacccacatgatggctcacagccatctgtaatttgagttccaagacatccaaaacccttttctgccctcagtcagcaccaggtatgcagtgcacatacttacatacatgcaggtaaactcaaaacacataaaaataaaacataaataaaaacagtaaaagtaaacccttaagaaacattctttttagccgggcgtggtggtgcacgcctttaatcccagcactcgggaggcagaggcaggcggatttctgagttcgaggccagcctggtctacaaagtgagttccaggacagccaggtctacacagagaaaccctgtctcgaaaaaatccaaaaagaataaaaagtattttcttagctgtaatttctagtaaaaaaaaaaaatgtagaaacccacccagaggtgtgtggtgatgcttttcttcccactgtgccgtcctgactccagctctgtagaccaggctggcctcccagcctgaactcagaaacctgcctgctatgcctcccaagtgctgttgttacaggcatatgccaacactcctgacggaggatccttatttttaagagaaaatacttttgtgggttttttcccctggcttaacagttttcctcatatctgggttcagtcaggtatgtcctgtgtatgtgttacccgtcctgacctgacaatgtcttgacaatggattcccttccctctgcttctctcgatttctctaacagtcagacccagtcttctgcaccgtggatgggacccaattacagtgtgccaccacctcaagggcagaatggcagcatgttgcctagtcagcctgctgggtatcgagtggccgggtatgaaacccagtgaaaacagactacagaacggaagccagtggcttgaagccacagggagggtaggaaaactgttgtttacgtaaagatttatcaaatcagtcagtacaaatgccagatacaatgtatttatttaaaagattcatttttttaatcatgaaattacttttcatccacattgttttaaaaaggaacacgatggtggatgtttgccaatttttgccttcattatcttttttgataaagtttctcagatccttgtttcaaagacaaatgcagggattactgccacttttttaaaaataagaggcagaaaattgcacaatgttgaacttttctccactaaaatcgtgtgcagttttagtttgcattcctgataggatttaaaacatgtaatatagatgtaaaaaaaaaaaaaaaaagccaaacttgtgcaaagtctagaagttctctatgactttcagtatgcacacagttgtcttaggtttaaagagtaatagacgtttcgtgaactataccatctccactattatccctttggaatttttaaatgtaaattattagtttatatcattttatatccacattttttcacaaatttgtcctgccttatgaaatatctgtaaaatattcttaaatgaaaaaaaatgatgttcacttagattgaaaacttttctcagatggaccgataattgcattcatcttgtttttataagagaaggtgcctcaaggattccctgttggatttgtttaaaaggactttttatcttctgtgataacctttgccatgtaccaggaactaggaaaacaagaaacttgttctaaagagatctgcaatgtgataagtcctcgccgcatcagtctcgtgcgtcagcatttatgtttacacgcattgttgtctaagatacttcagcaagtcatgttgtgcagttagcaaactttgtatgtcatttcctcctacaggtgtcatgaagagttgacgtaagacagattttacatttgcatgtgaatatcacatacatactttggtagtctttgatacaaactcatctgctcttctttttcaagcattttgagacacaaagttgtatgtaaaggaatatattaattagattttttttagttagatttactttaaaaagtaaatccactttaatgtgtacaaatgatagctgtgaaactgtagagtttttctctgtcaggcttgggattcactaagaactccaaaactagcctgaaggaccagccgggcaaaacattttttaaatattcagaggccaaaatatgagcaaacaaataaaaaaactcttaaaagtcctacctccttaaacagccttcaaggcaaagcccctgcgcacaggcatggctttggtcttggtccctggtctcttggagcccccatagtactattgaggtttactcttcactggcgttgttctagccttggggttcctaatatatttcagagtctcttggtttggtgtggttttagtgatgaccacaccttctcctcacagtagagaggaaggagccctgtgcactgtgggtttgaggcctgcagagagctcccagcattttacttgggcctatggaattggaagcatttcctggcctgtatctttcaggtaggagcacacaactctaacctgttttctaagccagtccccttgtacaacagagcccaggaagacagtctcaaatgtgtgcttctctggagctcaattctatgcagttgtgctaatgtttcattaagtcactgtgtatttttaagtactaaaacattgaaatttgctttatggaagatgaataaattttttaaatacttggaattttatttctttgtaaacttctacagatcaggacagacaataaaagcagcttaaataaaatactaaaaacaaaaagacaaaacccacacaccaggatgctatctttaagtttttctggatttgtctctattttagcatccttagtatatgtctttgtttaagaattttacttcctttgtaactttaggctgcaggatcttttgggcacaagtaatttattgtgtgcaaaagtgtagtttctctcttcttttgactctttcaagtactaaggacttttattagtagcatattcttattgcacagatcttaaaattcatgttgatttgctgttaagaaggtggtggtgctgtgttcctgatgaaagactgacatatcaggaacatagctgttgttagtttaaataaatcatttaagtcatttctcaacctaaaaaacttcatcctggtataagtatcaggtaaggaaatgaaggtttttaaagtgaacttccttgcaatatgcattcaggttttattctctggtactgtaaagaacctgagtgactctcagtgggtcatggagccagtattctttatcctgactgtttggatattaaaattcctttatgttttatatgacctgtggggtcttcttgcagtgattattgtgtgtgagatttttcccccggcgctggcgtattcatattatattgttaacactcaggttcaccttatcccagaagccatggtctgcatgggttccagaggatctgcactctgcagacttagccaactgtgaagtcagagtttggtcttcctggtgtaaaggagactccccgtctagattgtccttctcttgcttttccctgtgctcatcttccttttagatttgtgctagaggtccctttcattgtcaggggactagattacatctacagaaggagtagacagacttaataagggctatccatgctactgagatgatagtttagttagacaaacaaaatatattcataggaaaatttcaaaagcaaaactttttaatgagtgatgatttctaaaaactaatttttattggtctagtgacctggtttagcaggtaaggtgattgttgccaagcctgatgccctgagtttgaacatactagggagaagtagagaattaattctcacaagttatcctcgtccctccacatgtgtacaccctcccaccatggccacgtatatcaataaatctttaaaactgcc

SEQ ID NO: 4 - DNA, 19 bp, Homo sapiens
tgtgttttatatgagaagg

SEQ ID NO: 5 - DNA, 16 bp, Homo sapiens
agggagtgtagtaaag

SEQ ID NO: 6 - DNA, 20 bp, Homo sapiens
gaaattttaagaattagtgg

SEQ ID NO: 7 - DNA, 18 bp, Homo sapiens
ttgagaagtaattgttgg

SEQ ID NO: 8 - DNA, 18 bp, Homo sapiens
gatgaggttgtaaatcag

SEQ ID NO: 9 - DNA, 19 bp, Homo sapiens
ggaattttggagaaaaata

SEQ ID NO: 10 - DNA, 20 bp, Homo sapiens
ttatttgttggatgaatgag

SEQ ID NO: 11 - DNA, 20 bp, Homo sapiens
ggttttaggatgttttagtg

SEQ ID NO: 12 - DNA, 20 bp, Homo sapiens
ttaaagagtaaagaatggaa

SEQ ID NO: 13 - DNA, 20 bp, Homo sapiens
gattaggtagaatatagtgt

SEQ ID NO: 14 - DNA, 20 bp, Homo sapiens
aaattttttaatgggaaagg

SEQ ID NO: 15 - DNA, 19 bp, Homo sapiens
gtaatgttaaatggaaggt

SEQ ID NO: 16 - DNA, 20 bp, Homo sapiens
gtgtttgttgaatggtagat

SEQ ID NO: 17 - DNA, 17 bp, Homo sapiens
aggaagattaagttaca

SEQ ID NO: 18 - DNA, 20 bp, Homo sapiens
ataataataaggttaggatg

SEQ ID NO: 19 - DNA, 18 bp, Homo sapiens
taaataggaatgttaggg

SEQ ID NO: 20 - DNA, 18 bp, Homo sapiens
taaagattagattgaagg

SEQ ID NO: 21 - DNA, 17 bp, Homo sapiens
tgaggagtattcaaggt

SEQ ID NO: 22 - DNA, 17 bp, Homo sapiens
atttgggaggtagtgaa

SEQ ID NO: 23 - DNA, 20 bp, Homo sapiens
gtgattattgtgtgtgagat

SEQ ID NO: 24 - DNA, 19 bp, Homo sapiens
agtgattattgtgtgtgag

SEQ ID NO: 25 - DNA, 20 bp, Homo sapiens
ttgtatgtaaaggaatatat

SEQ ID NO: 26 - DNA, 20 bp, Homo sapiens
gttgtatgtaaaggaatata

SEQ ID NO: 27 - DNA, 20 bp, Homo sapiens
agttgtatgtaaaggaatat

SEQ ID NO: 28 - DNA, 20 bp, Homo sapiens
aagttgtatgtaaaggaata

SEQ ID NO: 29 - DNA, 20 bp, Homo sapiens
aaagttgtatgtaaaggaat

SEQ ID NO: 30 - DNA, 16 bp, Homo sapiens
gtggataaatgttggc

SEQ ID NO: 31 - DNA, 16 bp, Homo sapiens
agtggataaatgttgg

SEQ ID NO: 32 - DNA, 18 bp, Homo sapiens
tgaggtatggagttttag

SEQ ID NO: 33 - DNA, 16 bp, Homo sapiens
tggtgtaatgtctggg

SEQ ID NO: 34 - DNA, 18 bp, Homo sapiens
gaatggtgtaatgtctgg

SEQ ID NO: 35 - DNA, 17 bp, Homo sapiens
tgaatggtgtaatgtct

SEQ ID NO: 36 - DNA, 17 bp, Homo sapiens
tgaagggattactgttt

SEQ ID NO: 37 - DNA, 17 bp, Homo sapiens
agtgaagggattactgt

SEQ ID NO: 38 - DNA, 17 bp, Homo sapiens
aagtgaagggattactg

SEQ ID NO: 39 - DNA, 18 bp, Homo sapiens
taaagtgaagggattact

SEQ ID NO: 40 - DNA, 19 bp, Homo sapiens
atataaagtgaagggatta

SEQ ID NO: 41 - DNA, 20 bp, Homo sapiens
tgaatgtgtttgtgttaata

SEQ ID NO: 42 - DNA, 20 bp, Homo sapiens
atgattgaatgtgtttgtgt

SEQ ID NO: 43 - DNA, 20 bp, Homo sapiens
tatgattgaatgtgtttgtg

SEQ ID NO: 44 - DNA, 20 bp, Homo sapiens
atatgattgaatgtgtttgt

SEQ ID NO: 45 - DNA, 20 bp, Homo sapiens
gatatgattgaatgtgtttg

SEQ ID NO: 46 - DNA, 16 bp, Homo sapiens
agattaggatttgtca

SEQ ID NO: 47 - DNA, 17 bp, Homo sapiens
gataatgggtaaggtaa

SEQ ID NO: 48 - DNA, 18 bp, Homo sapiens
aagataatgggtaaggta

SEQ ID NO: 49 - DNA, 16 bp, Homo sapiens
tatggatgtaagggta

SEQ ID NO: 50 - DNA, 20 bp, Homo sapiens
ttatggatgtaagggtattt

SEQ ID NO: 51 - DNA, 17 bp, Homo sapiens
attatggatgtaagggt

SEQ ID NO: 52 - DNA, 18 bp, Homo sapiens
atgattatggatgtaagg

SEQ ID NO: 53 - DNA, 19 bp, Homo sapiens
aaatgattatggatgtaag

SEQ ID NO: 54 - DNA, 19 bp, Artificial sequence
  Synthetic oligonucleotide
ccttctcatataaaacaca

SEQ ID NO: 55 - DNA, 16 bp, Artificial sequence
  Synthetic oligonucleotide
ctttactacactccct

SEQ ID NO: 56 - DNA, 20 bp, Artificial sequence
  Synthetic oligonucleotide
ccactaattcttaaaatttc

SEQ ID NO: 57 - DNA, 18 bp, Artificial sequence
  Synthetic oligonucleotide
ccaacaattacttctcaa

SEQ ID NO: 58 - DNA, 18 bp, Artificial sequence
  Synthetic oligonucleotide
ctgatttacaacctcatc

SEQ ID NO: 59 - DNA, 19 bp, Artificial sequence
  Synthetic oligonucleotide
tatttttctccaaaattcc

SEQ ID NO: 60 - DNA, 20 bp, Artificial sequence
  Synthetic oligonucleotide
ctcattcatccaacaaataa

SEQ ID NO: 61 - DNA, 20 bp, Artificial sequence
  Synthetic oligonucleotide
cactaaaacatcctaaaacc

SEQ ID NO: 62 - DNA, 20 bp, Artificial sequence
  Synthetic oligonucleotide
ttccattctttactctttaa

SEQ ID NO: 63 - DNA, 20 bp, Artificial sequence
  Synthetic oligonucleotide
acactatattctacctaatc

SEQ ID NO: 64 - DNA, 20 bp, Artificial sequence
  Synthetic oligonucleotide
cctttcccattaaaaaattt

SEQ ID NO: 65 - DNA, 19 bp, Artificial sequence
  Synthetic oligonucleotide
accttccatttaacattac

SEQ ID NO: 66 - DNA, 20 bp, Artificial sequence
  Synthetic oligonucleotide
atctaccattcaacaaacac

SEQ ID NO: 67 - DNA, 17 bp, Artificial sequence
  Synthetic oligonucleotide
tgtaacttaatcttcct

SEQ ID NO: 68 - DNA, 20 bp, Artificial sequence
  Synthetic oligonucleotide
catcctaaccttattattat

SEQ ID NO: 69 - DNA, 18 bp, Artificial sequence
  Synthetic oligonucleotide
ccctaacattcctattta

SEQ ID NO: 70 - DNA, 18 bp, Artificial sequence
  Synthetic oligonucleotide
ccttcaatctaatcttta

SEQ ID NO: 71 - DNA, 17 bp, Artificial sequence
  Synthetic oligonucleotide
accttgaatactcctca

SEQ ID NO: 72 - DNA, 17 bp, Artificial sequence
  Synthetic oligonucleotide
ttcactacctcccaaat

SEQ ID NO: 73 - DNA, 20 bp, Artificial sequence
  Synthetic oligonucleotide
atctcacacacaataatcac

SEQ ID NO: 74 - DNA, 19 bp, Artificial sequence
  Synthetic oligonucleotide
ctcacacacaataatcact

SEQ ID NO: 75 - DNA, 20 bp, Artificial sequence
  Synthetic oligonucleotide
atatattcctttacatacaa

SEQ ID NO: 76 - DNA, 20 bp, Artificial sequence
  Synthetic oligonucleotide
tatattcctttacatacaac

SEQ ID NO: 77 - DNA, 20 bp, Artificial sequence
  Synthetic oligonucleotide
atattcctttacatacaact

SEQ ID NO: 78 - DNA, 20 bp, Artificial sequence
  Synthetic oligonucleotide
tattcctttacatacaactt

SEQ ID NO: 79 - DNA, 20 bp, Artificial sequence
  Synthetic oligonucleotide
attcctttacatacaacttt

SEQ ID NO: 80 - DNA, 16 bp, Artificial sequence
  Synthetic oligonucleotide
gccaacatttatccac

SEQ ID NO: 81 - DNA, 16 bp, Artificial sequence
  Synthetic oligonucleotide
ccaacatttatccact

SEQ ID NO: 82 - DNA, 18 bp, Artificial sequence
  Synthetic oligonucleotide
ctaaaactccatacctca

SEQ ID NO: 83 - DNA, 16 bp, Artificial sequence
  Synthetic oligonucleotide
cccagacattacacca

SEQ ID NO: 84 - DNA, 18 bp, Artificial sequence
  Synthetic oligonucleotide
ccagacattacaccattc

SEQ ID NO: 85 - DNA, 17 bp, Artificial sequence
  Synthetic oligonucleotide
agacattacaccattca

SEQ ID NO: 86 - DNA, 17 bp, Artificial sequence
  Synthetic oligonucleotide
aaacagtaatcccttca

SEQ ID NO: 87 - DNA, 17 bp, Artificial sequence
  Synthetic oligonucleotide
acagtaatcccttcact

SEQ ID NO: 88 - DNA, 17 bp, Artificial sequence
  Synthetic oligonucleotide
cagtaatcccttcactt

SEQ ID NO: 89 - DNA, 18 bp, Artificial sequence
  Synthetic oligonucleotide
agtaatcccttcacttta

SEQ ID NO: 90 - DNA, 19 bp, Artificial sequence
  Synthetic oligonucleotide
taatcccttcactttatat

SEQ ID NO: 91 - DNA, 20 bp, Artificial sequence
  Synthetic oligonucleotide
tattaacacaaacacattca

SEQ ID NO: 92 - DNA, 20 bp, Artificial sequence
  Synthetic oligonucleotide
acacaaacacattcaatcat

SEQ ID NO: 93 - DNA, 20 bp, Artificial sequence
  Synthetic oligonucleotide
cacaaacacattcaatcata

SEQ ID NO: 94 - DNA, 20 bp, Artificial sequence
  Synthetic oligonucleotide
acaaacacattcaatcatat

SEQ ID NO: 95 - DNA, 20 bp, Artificial sequence
  Synthetic oligonucleotide
caaacacattcaatcatatc

SEQ ID NO: 96 - DNA, 16 bp, Artificial sequence
  Synthetic oligonucleotide
tgacaaatcctaatct

SEQ ID NO: 97 - DNA, 17 bp, Artificial sequence
  Synthetic oligonucleotide
ttaccttacccattatc

SEQ ID NO: 98 - DNA, 18 bp, Artificial sequence
  Synthetic oligonucleotide
taccttacccattatctt

SEQ ID NO: 99 - DNA, 16 bp, Artificial sequence
  Synthetic oligonucleotide
tacccttacatccata

SEQ ID NO: 100 - DNA, 20 bp, Artificial sequence
  Synthetic oligonucleotide
aaatacccttacatccataa

SEQ ID NO: 101 - DNA, 17 bp, Artificial sequence
  Synthetic oligonucleotide
acccttacatccataat

SEQ ID NO: 102 - DNA, 18 bp, Artificial sequence
  Synthetic oligonucleotide
ccttacatccataatcat

SEQ ID NO: 103 - DNA, 19 bp, Artificial sequence
  Synthetic oligonucleotide
cttacatccataatcattt

(19) United States
(12) Patent Application Publication
(10) Pub. No.: US 2021/0261961 A1
(43) Pub. Date: Aug. 26, 2021

(54) ANTISENSE OLIGONUCLEOTIDES TARGETING TIA1

(71) Applicant: Hoffmann-La Roche Inc., Little Falls, NJ (US)

(72) Inventors: Veronica COSTA, Basel (CH); Heidi Rye HUDLEBUSCH, Brønshøj (DK); Ravi JAGASIA, Loerrach (DE); Dheeraj MALHOTRA, Basel (CH); Lykke PEDERSEN, Copenhagen NV (DK)

(21) Appl. No.: 17/245,443

(22) Filed: Apr. 30, 2021

Related U.S. Application Data
(63) Continuation of application No. PCT/EP2019/079583, filed on Oct. 30, 2019.

(30) Foreign Application Priority Data
Nov. 1, 2018 (EP) 18203935.4

(51) Int. Cl. C12N 15/113

(52) U.S. Cl. CPC C12N 15/113; C12N 2310/11; C12N 2310/3231; C12N 2310/341; C12N 2310/351; C12N 2310/346; C12N 2310/3341; C12N 2310/315

(57) ABSTRACT

The present invention relates to antisense oligonucleotides (oligomers) complementary to nucleic acids encoding mammalian T cell-restricted intracellular antigen-1 (TIA1), in particular antisense oligonucleotides targeting TIA1 pre-mRNA sequences, which are capable of inhibiting the expression of TIA1. Inhibition of TIA1expression is beneficial for a range of medical disorders including neurodegenerative diseases, such as amyotrophic lateral sclerosis (ALS) or Frontotemporal Dementia.

### SEQUENCE LISTING

The instant application contains a Sequence Listing which has been submitted electronically in ASCII format and is hereby incorporated by reference in its entirety. Said ASCII copy, created on Mar. 29, 2021 is named 51551-006001_Sequence_Listing_03.29.21_ST25 and is 156,502 bytes in size.

### FIELD OF INVENTION

The present invention relates to antisense oligonucleotides (oligomers) complementary to nucleic acids encoding mammalian T cell-restricted intracellular antigen-1 (TIA1), in particular antisense oligonucleotides targeting TIA1 pre-mRNA sequences, which are capable of inhibiting the expression of TIA1. Inhibition of TIA 1expression is beneficial for a range of medical disorders including neurodegenerative diseases, such as amyotrophic lateral sclerosis (ALS), Frontotemporal Dementia, and tauopathies.

### BACKGROUND

One of the hallmarks of many neurodegenerative diseases is the accumulation of protein inclusions in the brain and central nervous system. These inclusions are insoluble aggregates of proteins and other cellular components that cause damage to cells and result in impaired function. Proteins such as tau, alpha-synuclein, huntingtin and P-amyloid have all been found to form inclusions in the brain and are linked to the development of a number of neurodegenerative diseases, including Alzheimer's disease and Huntington's disease. Neurodegenerative diseases are also associated with stress granules, which contain RNAs and aggregated RNA binding proteins.

T cell-restricted intracellular antigen-1 (TIA-1) is an RNA binding protein and a core nucleating stress granule protein. In stress granule formation, nucleation is followed by recruitment of secondary RNA-binding proteins to form a mature stress granule, which is a key component of stress-induced translational suppression. TIA1 co-localizes with neuropathology in the brain tissue of subjects with neurodegenerative disorders (see for example Maziuk et al., Acta Neuropathologica Communications 2018 6:71). Appicco et al., Nat Neurosci. 2018 Jan; 21(1):72-80 reports that reducing the RNA binding protein TIA1 protects against tau-mediated neurodegeneration in vivo.

Amyotrophic lateral sclerosis (ALS) is a complex neurodegenerative disease, characterized genetically by a disproportionately large contribution of rare genetic variation. Driven by advances in massive parallel sequencing and applied on large patient-control cohorts, systematic identification of these rare variants that make up the genetic architecture of ALS became feasible (Nguyen et al., Trends in Genetics June 2018, Vol. 34, No. 6). Mackenzie et al., Neuron 95, 808-816, Aug. 16, 2017 reports that mutations affecting the low-complexity domain of TIA1 cause Amyotrophic Lateral Sclerosis (ALS) and Frontotemporal Dementia (FTD) ALS and ALS-FTD and that ALS-linked TIA1 mutations share a neuropathological TDP-43 signature, that TIA1 mutations promote phase separation and impair stress granule dynamics, and that TDP-43 recruited to poorly dynamic stress granules becomes immobile and insoluble attributing to disease. Hirsch-Reinshagen et al. Acta Neuropathologica Communications (2017) 5:96 discloses clinical and widespread TDP-43 neuropathological features of ALS/FTD with Tia1 mutations.

WO 2017/066657 refers to nucleic acid based inhibitors of TIA1.

### OBJECTIVE OF THE INVENTION

The inventors have identified particularly effective regions of the TIA1 transcript (TIA1) for antisense inhibition in vitro or in vivo, and provides for antisense oligonucleotides, including LNA gapmer oligonucleotides, which target these regions of the TIA1 prem RNA or mature mRNA. The present invention identifies oligonucleotides which inhibit human TIA1 which are useful in the treatment of a range of medical disorders including neurological disorders, particularly nuerological disorders associated with stress granule formation.

### STATEMENT OF THE INVENTION

The invention provides for an antisense oligonucleotide, 10-30 nucleotides in length, targeting a human TIA1 target nucleic acid. The invention provides a range of novel target sites within the human TIA1 pre-mRNA, and further provides for antisense oligonucleotides which comprise at least 10 or more contiguous nucleotides which are complementary to such a novel target site. The antisense oligonucleotides of the invention are capable of inhibiting the expression of human TIA1 in a cell which is expressing human TIA1.

The invention provides for an antisense oligonucleotide, 10-30 nucleotides in length, targeting a human TIA1 target nucleic acid, wherein the antisense oligonucleotide is capable of inhibiting the expression of human TIA1 in a cell which is expressing human TIA1.

The invention provides for an antisense oligonucleotide, 10-30 nucleotides in length, targeting a human TIA1 target nucleic acid, wherein said antisense oligonucleotide comprises a contiguous nucleotide sequence 10-30 nucleotides in length, wherein the contiguous nucleotide sequence is at least 90% complementary, such as fully complementary, to a sequence selected from the group consisting of SEQ ID NO 4-53.

The invention provides for an antisense oligonucleotide, 10-30 nucleotides in length, targeting a human TIA1 target nucleic acid, wherein said antisense oligonucleotide comprises a contiguous nucleotide sequence 10-30 nucleotides in length, wherein the contiguous nucleotide sequence is at least 90% complementary, such as fully complementary to a region of SEQ ID NO 1 selected from the group consisting of (target sequence regions—identified by their nucleotide position range in SEQ ID NO 1)-LIST A: 8-23; 33-52; 54-96; 103-139; 148-162; 164-195; 212-358; 360-393; 403-423; 456-478; 491-507; 509-538; 571-606; 604-627; 637-658; 660-685; 687-712; 714-729; 744-765; 792-843; 845-873; 875-916; 931-950; 955-971; 973-991; 1003-1029; 1045-1081; 1083-1101; 1105-1150; 1153-1288; 1297-1318; 1331-1368; 1370-1389; 1391-1465; 1482-1521; 1523-1557; 1557-1579; 1591-1605; 1613-1669; 1678-1698; 1743-1787; 1789-1816; 1822-1855; 1860-1892; 1901-1918; 1908-1929; 1919-1961; 1964-1987; 1978-2002; 2004-2018; 2020-2049; 2038-2052; 2048-2068; 2070-2086; 2088-2115; 2117-2134; 2136-2166; 2167-2207; 2209-2224; 2228-2260; 2268-2337; 2341-2362; 2372-2387; 2389-2403; 2413-2468; 2463-2480; 2503-2537; 2532-2547; 2541-2558; 2550-2573; 2579-2624; 2614-2634; 2626-2644; 2645-2671; 2669-2695; 2700-2718; 2712-2747; 2755-2780; 2825-2876; 2878-2904; 2906-2935; 2940-2961; 2963-3006; 3008-3035; 3042-3056; 3067-3086; 3090-3106; 3117-3135; 3137-3213; 3220-3272; 3280-3305; 3342-3440; 3430-3448; 3442-3461; 3468-3514; 3516-3544; 3583-3635; 3651-3678; 3709-3730; 3732-3756; 3755-3783; 3791-3805; 3800-3833; 3840-3859; 3871-3889; 3878-3898; 3903-3932; 3934-3952; 3949-3963; 3958-3987; 4002-4044; 4046-4060; 4052-4088; 4104-4130; 4134-4248; 4251-4270; 4302-4324; 4348-4383; 4385-4455; 4457-4529; 4531-4551; 4553-4625; 4656-4694; 4700-4717; 4738-4771; 4775-4790; 4792-4837; 4839-4901; 4908-4959; 4961-5004; 5040-5085; 5087-5147; 5149-5170; 5172-5203; 5211-5225; 5215-5230; 5227-5256; 5270-5284; 5285-5311; 5320-5335; 5325-5341; 5340-5356; 5364-5388; 5384-5437; 5439-5484; 5482-5515; 5537-5560; 5562-5610; 5622-5670; 5672-5692; 5714-5779; 5793-5812; 5814-5888; 5895-5931; 5933-5959; 5961-5975; 6002-6016; 6026-6064; 6135-6171; 6177-6195; 6208-6232; 6272-6299; 6315-6341; 6355-6401; 6408-6428; 6430-6452; 6461-6483; 6503-6571; 6573-6635; 6639-6659; 6661-6685; 6690-6708; 6729-6747; 6739-6757; 6792-6892; 6894-6913; 6915-6960; 6950-6967; 6976-6994; 7002-7031; 7033-7047; 7056-7095; 7115-7151; 7153-7174; 7203-7220; 7230-7244; 7264-7293; 7284-7318; 7319-7348; 7359-7402; 7405-7442; 7452-7474; 7466-7487; 7508-7524; 7529-7544; 7561-7611; 7635-7655; 7657-7681; 7675-7701; 7713-7732; 7748-7774; 7766-7783; 7787-7829; 7841-7863; 7866-7890; 7899-7920; 7922-7936; 7954-8016; 8022-8058; 8073-8088; 8097-8119; 8140-8155; 8145-8189; 8191-8208; 8220-8241; 8243-8290; 8292-8308; 8331-8401; 8403-8423; 8425-8441; 8454-8494; 8496-8525; 8527-8549; 8552-8574; 8576-8604; 8663-8720; 8728-8771; 8774-8825; 8856-8922; 8924-8942; 8945-8982; 9007-9041; 9058-9077; 9099-9149; 9156-9171; 9173-9187; 9189-9207; 9224-9243; 9241-9277; 9270-9289; 9279-9299; 9302-9323; 9328-9366; 9357-9401; 9396-9412; 9403-9419; 9413-9427; 9421-9443; 9432-9473; 9477-9529; 9586-9601; 9603-9618; 9621-9635; 9625-9648; 9638-9653; 9644-9658; 9650-9693; 9699-9741; 9757-9786; 9791-9820; 9846-9870; 9862-9881; 9897-9924; 9964-9983; 9985-10052; 10054-10090; 10092-10171; 10172-10190; 10182-10199; 10189-10207; 10201-10241; 10231-10253; 10265-10303; 10293-10314; 10331-10351; 10372-10403; 10432-10459; 10486-10500; 10499-10513; 10506-10520; 10510-10526; 10528-10550; 10547-10564; 10588-10614; 10616-10639; 10633-10655; 10683-10728; 10726-10777; 10782-10796; 10814-10844; 10854-10869; 10860-10887; 10883-10898; 10906-10925; 10930-10951; 10942-10978; 10981-10996; 11027-11068; 11070-11135; 11140-11163; 11193-11208; 11210-11245; 11264-11305; 11297-11311; 11324-11377; 11386-11415; 11404-11440; 11432-11447; 11449-11463; 11477-11492; 11493-11530; 11590-11613; 11615-11632; 11634-11674; 11694-11710; 11743-11757; 11746-11789; 11793-11810; 11800-11840; 11858-11914; 11908-11925; 11921-11936; 11938-11952; 11987-12002; 12024-12047; 12092-12143; 12145-12164; 12167-12191; 12193-12214; 12223-12272; 12266-12294; 12312-12353; 12355-12375; 12386-12400; 12416-12431; 12439-12461; 12472-12624; 12642-12662; 12673-12692; 12685-12701; 12694-12729; 12748-12772; 12788-12803; 12823-12839; 12841-12856; 12864-12897; 12899-12915; 12926-12954; 12956-12983; 12985-13023; 13025-13054; 13130-13147; 13158-13181; 13190-13215; 13237-13279; 13304-13320; 13311-13331; 13322-13341; 13343-13377; 13384-13404; 13406-13424; 13418-13444; 13456-13493; 13493-13507; 13524-13549; 13551-13586; 13588-13602; 13620-13651; 13659-13710; 13723-13760; 13764-13778; 13781-13808; 13806-13821; 13810-13864; 13865-13879; 13886-13901; 13894-13910; 13903-13927; 13955-13982; 13998-14013; 14016-14037; 14040-14060; 14878-14900; 14898-14945; 14941-14958; 14960-14980; 14982-15002; 14997-15014; 15003-15026; 15028-15043; 15058-15076; 15068-15089; 15090-15106; 15128-15164; 15408-15423; 15422-15437; 15439-15458; 15468-15483; 15474-15491; 15495-15516; 15526-15560; 15562-15616; 15618-15632; 15634-15655; 15675-15692; 15704-15730; 15732-15777; 15783-15800; 15814-15837; 15829-15856; 15845-15860; 15862-15876; 15881-15905; 15921-15939; 15941-15988; 15990-16004; 15995-16022; 16011-16027; 16019-16035; 16045-16060; 16078-16092; 16086-16127; 16127-16165; 16197-16301; 16293-16309; 16301-16331; 16324-16344; 16347-16390; 16404-16418; 16411-16459; 16474-16497; 16499-16514; 16530-16557; 16548-16597; 16599-16645; 16661-16676; 16678-16692; 16702-16729; 16741-16759; 16774-16798; 16810-16865; 16867-16937; 16944-16966; 16988-17021; 17023-17041; 17043-17067; 17088-17124; 17126-17168; 17171-17212; 17201-17219; 17208-17223; 17225-17315; 17307-17325; 17355-17371; 17365-17380; 17381-17404; 17416-17433; 17429-17444; 17435-17472; 17506-17525; 17528-17544; 17559-17585; 17588-17633; 17624-17638; 17641-17663; 17678-17742; 17732-17862; 17864-17942; 17980-17996; 17992-18012; 18004-18018; 18019-18047; 18038-18053; 18057-18083; 18074-18125; 18118-18135; 18140-18163; 18165-18179; 18180-18197; 18194-18221; 18214-18241; 18272-18329; 18331-18350; 18353-18369; 18379-18403; 18396-18422; 18432-18457; 18464-18490; 18502-18537; 18526-18560; 18564-18588; 18592-18606; 18603-18622; 18648-18673; 18675-18696; 18712-18727; 18717-18731; 18741-18796; 18798-18818; 18845-18861; 18860-18892; 18881-18896; 18886-18904; 18909-18952; 18949-18963; 18966-18980; 18999-19013; 19003-19021; 19023-19038; 19040-19056; 19058-19093; 19123-19139; 19153-19179; 19189-19206; 19208-19269; 19284-19298; 19300-19337; 19332-19371; 19373-19421; 19438-19664; 19666-19696; 19698-19726; 19752-19767; 19776-19805; 19817-19862; 19860-19880; 19887-19949; 19953-19989; 19991-20118; 20120-20439; 20441-20548; 20550-20575; 20579-20642; 20644-20687; 20689-20738; 20740-20869; 20871-20998; 21029-21080; 21082-21170; 21196-21218; 21234-21252; 21274-21322; 21324-21351; 21379-21443; 21464-21497; 21499-21574; 21600-21617; 21638-21665; 21669-21691; 21693-21751; 21811-21847; 21849-21870; 21872-21893; 21895-21930; 21935-21974; 21976-21995; 21997-22017; 22019-22036; 22044-22102; 22107-22143; 22154-22180; 22179-22199; 22202-22222; 22224-22238; 22240-22286; 22288-22304; 22313-22340; 22342-22367; 22382-22409; 22421-22445; 22461-22497; 22500-22529; 22531-22583; 22597-22648; 22653-22675; 22681-22716; 22718-22768; 22770-22787; 22790-22821; 22835-22856; 22858-22878; 22880-22923; 22934-22948; 22950-23417; 23419-23446; 23460-23547; 23562-23622; 23624-23783; 23785-23821; 23823-23940; 23942-23957; 23959-23973; 23975-23991; 23993-24116; 24134-24158; 24223-24238; 24239-24254; 24254-24278; 24278-24295; 24298-24316; 24318-24412; 24418-24447; 24451-24476; 24475-24496; 24492-24527; 24558-24585; 24581-24597; 24593-24618; 24607-24622; 24633-24658; 24660-24681; 24675-24700; 24702-24731; 24734-24748; 24743-24757; 24759-24779; 24794-24837; 24868-24899; 24894-24909; 24934-24967; 24957-24991; 25012-25090; 25092-25116; 25118-25143; 25154-25170; 25181-25204; 25213-25233; 25253-25276; 25278-25318; 25309-25324; 25326-25345; 25344-25363; 25394-25415; 25439-25496; 25498-25514; 25571-25591; 25608-25640; 25660-25678; 25680-25703; 25705-25738; 25738-25759; 25759-25781; 25794-25816; 25818-25842; 25833-25875; 25889-25921; 25911-25926; 25930-25958; 25953-25970; 25964-25979; 25990-26023; 26027-26066; 26070-26087; 26089-26103; 26122-26144; 26146-26161; 26187-26241; 26243-26262; 26276-26300; 26302-26332; 26332-26356; 26358-26391; 26388-26451; 26453-26480; 26516-26536; 26538-26583; 26586-26601; 26590-26628; 26617-26652; 26659-26673; 26674-26700; 26709-26724; 26726-26752; 26778-26812; 26814-26841; 26839-26862; 26852-26872; 26877-26898; 26900-26923; 26925-26996; 26996-27020; 27053-27096; 27095-27110; 27123-27162; 27167-27181; 27175-27190; 27200-27253; 27244-27258; 27255-27269; 27260-27274; 27276-27297; 27312-27335; 27337-27356; 27357-27378; 27380-27399; 27425-27442; 27435-27456; 27449-27465; 27455-27481; 27481-27505; 27523-27560; 27553-27568; 27571-27591; 27592-27622; 27624-27641; 27645-27660; 27694-27720; 27725-27772; 27785-27811; 27813-27828; 27848-27881; 27885-27905; 27907-27922; 27933-28001; 28003-28043; 28059-28082; 28098-28145; 28148-28216; 28219-28235; 28267-28288; 28284-28318; 28321-28343; 28351-28378; 28387-28407; 28402-28431; 28433-28454; 28443-28470; 28471-28485; 28481-28495; 28494-28509; 28511-28527; 28564-28589; 28591-28613; 28615-28642; 28644-28662; 28679-28706; 28719-28735; 28754-28773; 28775-28801; 28803-28841; 28838-28852; 28867-28884; 28889-28905; 28900-28925; 28953-28984; 29039-29072; 29074-29114; 29126-29153; 29155-29172; 29190-29234; 29236-29265; 29270-29302; 29330-29372; 29394-29478; 29489-29523; 29525-29543; 29558-29623; 29625-29654; 29670-29724; 29724-29744; 29750-29773; 29811-29861; 29893-29911; 29938-29958; 29949-29976; 29978-30005; 30020-30036; 30030-30052; 30052-30075; 30070-30097; 30138-30160; 30149-30164; 30168-30189; 30223-30292; 30306-30335; 30337-30357; 30363-30383; 30395-30409; 30412-30442; 30454-30471; 30488-30523; 30524-30556; 30565-30609; 30611-30645; 30650-30732; 30730-30746; 30748-30792; 30804-30822; 30834-30854; 30864-30885; 30887-30933; 30944-30964; 30957-30972; 30969-30994; 30985-31005; 31017-31061; 31073-31097; 31104-31121; 31123-31143; 31137-31177; 31174-31206; 31210-31239; 31240-31255; 31247-31269; 31262-31277; 31270-31292; 31288-31340; 31342-31360; 31358-31398; 31388-31403; 31401-31417; 31446-31465; 31478-31492; 31555-31578; 31580-31606; 31608-31654; 31659-31825; 31827-31862; 31864-31898; 31900-31939; 31941-31971; 31973-32019; 32021-32041; 32043-32096; 32116-32275; 32277-32342; 32354-32375; 32377-32444; 32446-32476; 32479-32529; 32566-32595; 32619-32636; 32630-32705; 32707-32739; 32764-32787; 32779-32850; 32845-32887; 32889-32926; 32932-32955; 32957-32987; 33009-33051; 33071-33103; 33108-33345; 33347-33483; 33500-33542; 33544-33579; 33581-33644; 33646-33686; 33679-33707; 33709-33790; 33792-33818; 33820-33856; 33858-33889; 33891-33910; 33912-33954; 33956-33980; 34002-34030; 34033-34086; 34088-34107; 34109-34126; 34131-34186; 34188-34207; 34209-34324; 34329-34360; 34364-34397; 34399-34413; 34416-34435; 34448-34490; 34492-34517; 34519-34536; 34538-34576; 34578-34592; 34607-34630; 34640-34670; 34672-34686; 34688-34703; 34729-34745; 34740-34773; 34773-34790; 34792-34809; 34804-34821; 34824-34861; 34872-34891; 34882-34902; 34893-34945; 34950-34971; 34973-34991; 34985-35003; 35006-35021; 35038-35061; 35062-35076; 35077-35123; 35141-35166; 35167-35185; 35205-35230; 35233-35257; 35259-35282; 35296-35318; 35327-35360; 35369-35408; 35423-35452; 35454-35479; 35543-35597; 35614-35649; 35643-35657; 35699-35735; 35743-35898; 35900-35943; 35945-36269; 36271-36285; 36287-36344; 36347-36370; 36374-36417; 36411-36458; 36460-36486; 36489-36656; 36658-36678; 36693-36895; 36918-37046; 37061-37096; 37098-37133; 37135-37199; 37201-37233; 37242-37272; 37279-37309; 37311-37337; 37339-37353; 37355-37415; 37419-37438; 37440-37458; 37477-37497; 37504-37523; 37541-37559; 37561-37596; 37598-37627; 37632-37676; 37712-37746; 37749-37769; 37770-37849; 37851-37903; 37905-37956; 37958-37972; 37974-38002; 38004-38082; 38099-38148; 38150-38174; 38177-38221; 38221-38255; 38257-38300; 38308-38380; 38396-38457; 38495-38509; and 38540-38554.

The invention provides for an antisense oligonucleotide, 10-30 nucleotides in length, targeting a human TIA1 target nucleic acid, wherein said antisense oligonucleotide comprises a contiguous nucleotide sequence 10-30 nucleotides in length, wherein the contiguous nucleotide sequence is at least 90% complementary, such as fully complementary to a region of SEQ ID NO 1 selected from the group consisting of (target sequence regions identified by their nucleotide position range in SEQ ID NO 1) LIST B: 26-44; 49-63; 183-202; 222-266; 275-297; 332-353; 376-390; 2019-2035; 2721-2746; 3802-3827; 4069-4085; 6139-6167; 6372-6388; 6739-6755; 9403-9418; 9854-9872; 10374-10400; 10633-10648; 10743-10759; 11040-11056; 11392-11406; 11502-11518; 12483-12503; 12506-12524; 12535-12595; 12607-12624; 12613-12627; 12687-12701; 12753-12768; 12881-12895; 13088-13120; 13604-13629; 14881-14897; 15136-15152; 15429-15443; 15782-15796; 16086-16121; 16364-16380; 16418-16432; 16425-16439; 16433-16453; 16702-16718; 17341-17360; 17490-17504; 17605-17621; 17852-17868; 17888-17915; 17992-18009; 18402-18420; 18703-18717; 19155-19171; 19333-19347; 19349-19386; 19467-19490; 19492-19507; 19510-19628; 19635-19662; 19811-19834; 19887-19910; 20126-20142; 20155-20182; 20184-20201; 20203-20346; 20366-20386; 20400-20414; 20747-20767; 20801-20816; 20835-20851; 20904-20923; 21048-21064; 22992-23008; 23014-23030; 23056-23072; 23093-23108; 23123-23138; 23188-23215; 23217-23368; 23502-23525; 23666-23691; 23745-23767; 23840-23865; 23896-23911; 24026-24040; 24042-24058; 24066-24113; 24713-24727; 24901-24915; 25989-26005; 26508-26525; 26514-26536; 26527-26541; 26842-26856; 28243-28260; 28487-28507; 28901-28925; 31247-31266; 31688-31752; 31754-31782; 31852-31885; 31904-31924; 31951-31966; 32179-32195; 32361-32393; 32395-32426; 33144-33159; 33173-33196; 33198-33229; 33261-33277; 33282-33300; 33840-33854; 33993-34007; 34002-34029; 34135-34150; 34170-34186; 34194-34231; 35167-35181; 35298-35319; 35880-35896; 35908-35923; 35925-35943; 35962-35976; 36011-36034; 36043-36077; 36073-36093; 36095-36116; 36136-36156; 36158-36193; 36195-36209; 36229-36251; 36287-36304; 36306-36325; 36472-36486; 36508-36525; 36563-36577; 36579-36605; 36608-36627; 36637-36652; 36658-36680; 36861-36889; 36956-36978; 37010-37050; 37099-37127; 37173-37187; 37417-37434; 37715-37746; 37751-37779; 37798-37813; 37815-37836; 37853-37869; 37880-37894; 38279-38296; 38352-38373; 38422-38436; 38467-38500; and 38720-38735.

The invention provides for an LNA antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a sequence selected from the group consisting of SEQ ID NO 4-53, wherein the antisense oligonucleotide is capable of inhibiting the expression of human TIA1 in a cell which is expressing human TIA1.

The invention provides for a gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a SEQ ID selected from the group consisting of SEQ ID NO 4-53 wherein the antisense oligonucleotide is capable of inhibiting the expression of human TIA1 in a cell which is expressing human TIA1.

The invention provides for an LNA gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a SEQ ID selected from the group consisting of SEQ ID NO 4-53 wherein the antisense oligonucleotide is capable of inhibiting the expression of human TIA1 in a cell which is expressing human TIA1.

The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a sequence shown in SEQ ID NO 11 or shown in SEQ ID NO 12, wherein the antisense oligonucleotide is capable of inhibiting the expression of human TIA1 in a cell which is expressing human TIA1.

The invention provides for an LNA antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a sequence shown in SEQ ID NO 11 or shown in SEQ ID NO 12, wherein the antisense oligonucleotide is capable of inhibiting the expression of human TIA1 in a cell which is expressing human TIA1.

The invention provides for a gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to a sequence shown in SEQ ID NO 11 or shown in SEQ ID NO 12, wherein the antisense oligonucleotide is capable of inhibiting the expression of human TIA1 in a cell which is expressing human TIA1.

The invention provides for an LNA gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a sequence shown in SEQ ID NO 11 or shown in SEQ ID NO 12, wherein the antisense oligonucleotide is capable of inhibiting the expression of human TIA1 in a cell which is expressing human TIA1.

The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to SEQ ID NO 11, wherein the antisense oligonucleotide is capable of inhibiting the expression of human TIA1 transcript in a cell which is expressing human TIA1 transcript.

The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to SEQ ID NO 12, wherein the antisense oligonucleotide is capable of inhibiting the expression of human TIA1 transcript in a cell which is expressing human TIA1 transcript.

The oligonucleotide of the invention as referred to or claimed herein may be in the form of a pharmaceutically acceptable salt.

The invention provides for a conjugate comprising the oligonucleotide according to the invention, and at least one conjugate moiety covalently attached to said oligonucleotide. The invention provides for a pharmaceutical composition comprising the oligonucleotide or conjugate of the invention and a pharmaceutically acceptable diluent, solvent, carrier, salt and/or adjuvant.

The invention provides for an in vivo or in vitro method for modulating TIA1 expression in a target cell which is expressing TIA1, said method comprising administering an oligonucleotide or conjugate or pharmaceutical composition of the invention in an effective amount to said cell. The invention provides for a method for treating or preventing a disease comprising administering a therapeutically or prophylactically effective amount of an oligonucleotide, conjugate or the pharmaceutical composition of the invention to a subject suffering from or susceptible to the disease.

In some embodiments, the disease is a neurodegenerative disease, such as a neurodegenerative disease.

In some embodiments, the disease is selected from the group consisting of Amyotrophic Lateral Sclerosis (ALS), Frontotemporal Dementia (FTD), tauopathies, such as primary tauopathies, frontotemporal dementia with parkinsonism (FTDP-17), frontotemporal lobar dementia (FTLD-TDP), Huntington's disease, Creutzfeld-Jacob disease, and spinomuscular atrophy, motor neuron disease, Tauopathy, Alzheimer's disease, and Welander distal myopathy.

In some embodiments, the disease is Amyotrophic Lateral Sclerosis.

In some embodiments, the disease is a tauopathies, such as a primary tauopathies,

In some embodiments, the disease is Frontotemporal Dementia (FTD). The invention provides for the oligonucleotide, conjugate or the pharmaceutical composition of the invention for use in medicine.

The invention provides for the oligonucleotide, conjugate or the pharmaceutical composition of the invention for use in the treatment or prevention of a neurodegenerative disease. The invention provides for the use of the oligonucleotide, conjugate or the pharmaceutical composition of the invention, for the preparation of a medicament for treatment or prevention of a neurodegenerative disease.

### DEFINITIONS

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. Adventurously, the contiguous nucleotide sequence is 100% complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified internucleoside linkages The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2 742 135 may for example be tolerated in an otherwise DNA phosphorothioate gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous sequence of nucleotides, at a given position of a separate nucleic acid molecule (e.g. the target nucleic acid or target sequence). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (e.g. in the contiguous nucleotide sequence of the compound of the invention and in the reference sequence), dividing that number by the total number of nucleotides in the aligned region and multiplying by 100.

Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003,Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965,Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG° . The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Target Nucleic Acid

According to the present invention, the target nucleic acid is a nucleic acid which encodes mammalian TIA1 and may for example be a gene, a TIA1 RNA, a mRNA, a pre-mRNA, a mature mRNA or a cDNA sequence. The target may therefore be referred to as a TIA1 target nucleic acid.

Suitably, the target nucleic acid encodes an TIA1 protein, in particular mammalian TIA1, such as the human TIA1 gene encoding pre-mRNA or mRNA sequences provided herein as SEQ ID NO 1.

In some embodiments the target may be the cynomolgus monkey TIA1 pre-mRNA, illustrated herein as SEQ ID NO 2, or the mouse TIA1 pre-mRNA, illustrated herein as SEQ ID NO 3. It will be recognized that the target sites identified by the inventors may be present in both SEQ ID NO 1, and SEQ ID NO 2 or SEQ ID NO 3.

4-53.

In some embodiments, the target nucleic acid is selected from the group consisting of SEQ ID NO 1, 2 or 3, or naturally occurring variants thereof (e.g. TIA1 sequences encoding a mammalian TIA1 protein).

If employing the oligonucleotide of the invention in research or diagnostics the target nucleic acid may be a cDNA or a synthetic nucleic acid derived from DNA or RNA. For in vivo or in vitro application, the oligonucleotide of the invention is typically capable of inhibiting the expression of the TIA1 target nucleic acid in a cell which is expressing the TIA1 target nucleic acid.

In some embodiments, the target cell, or the cell which is expressing human TIA1 is an in vitro cell line or cell culture—see the examples for a list of suitable cell lines. In some embodiments the cell which is expressing human TIA1 is a U2OS cell or an iPSC-derived motor neuron cell. In some embodiments, the target cell is a motor neuron, such as an upper or lower motor neuron (for example may be the target cell for compounds for treatment of ALS). In some embodiments, the target cell is a cortical neuron (for example may be the target cell for compounds for treatment of FTD or tauopathies, such as primary tauopathies). The contiguous sequence of nucleobases of the oligonucleotide of the invention is typically complementary to the TIA1 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid is a messenger RNA, such as a mature mRNA or a pre-mRNA which encodes mammalian TIA1 protein, such as human TIA1, e.g. the human TIA1 pre-mRNA sequence, such as that disclosed as SEQ ID NO 1.sequences—it will be understood that target RNA sequences have uracil (U) bases in place of the thymidine bases (T).

Exemplary Target Nucleic Acids

| | RNA | SEQ |
|---|---|---|
| Species | type | ID NO |
| Human | pre-mRNA | 1 |
| Cynomolgus monkey | pre-mRNA | 2 |
| Mouse | pre-mRNA | 3 |

| | | | Genomic | | | | |
|---|---|---|---|---|---|---|---|
| | | | Coordinates | | | | |
| Species | Chr | Strand | Start | End | Assembly | Ensembl gene ID | SEQ ID NO |
| Human | 2 | Rev | 70209444 | 70248660 | GRCh38 | ENSG00000116001 | 1 |
| Cynomolgus | 13 | Fwd | 39655996 | 39693983 | Macaca_fascicularis_5.0 | ENSMFAG00000045951 | 2 |
| monkey | | | | | | | |
| Mouse | 6 | Fwd | 86404219 | 86433405 | GRCm38 | ENSMUSG00000071337 | 3 |

In some embodiments, the oligonucleotide of the invention targets SEQ ID NO 1: In some embodiments, the oligonucleotide of the invention is complementary to SEQ ID NO 1, and is capable of inhibiting the expression of the human TIA1 pre-mRNA, in a cell which is expressing human TIA1 pre-mRNA.

In some embodiments, the oligonucleotide of the invention targets SEQ ID NO 2. In some embodiments, the oligonucleotide of the invention is complementary to SEQ ID NO 2, and is capable of inhibiting the expression of the Cynomolgus monkey TIA1 pre-mRNA, in a cell which is expressing Cynomolgus monkey TIA1 pre-mRNA.

In some embodiments, the oligonucleotide of the invention targets SEQ ID NO 3. In some embodiments, the oligonucleotide of the invention is complementary to SEQ ID NO 3, and is capable of inhibiting the expression of the mouse TIA1 pre-mRNA, in a cell which is expressing the mouse TIA1 pre-mRNA.

Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid which is complementary to the contiguous nucleotide sequence of the oligonucleotide of the invention.

Herein are provided numerous target sequence regions, as defined by regions of the human TIA1 pre-mRNA (using SEQ ID NO 1 as a reference) which may be targeted by the oligonucleotides of the invention.

In some embodiments the target sequence is longer than the complementary sequence of a single oligonucleotide, and may, for example represent a preferred region of the target nucleic acid which may be targeted by several oligonucleotides of the invention.

The oligonucleotide of the invention comprises a contiguous nucleotide sequence which is complementary to or hybridizes to the target nucleic acid, such as a sub-sequence of the target nucleic acid, such as a target sequence described herein. The oligonucleotide comprises a contiguous nucleotide sequence which are complementary to a target sequence present in the target nucleic acid molecule. The contiguous nucleotide sequence (and therefore the target sequence) comprises of at least 10 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

Target Sequence Regions

The inventors have identified particularly effective sequences of the TIA1 target nucleic acid which may be targeted by the oligonucleotide of the invention—these are referred to as target sites or target sequence regions. The antisense oligonucleotide of the invention may therefore comprises a contiguous nucleotide sequence which comprises at least 10 contiguous nucleotides, such as at least 12 contiguous nucleotides, which are complementary to, such as fully complementary to the target site region.

In some embodiments, the target site region is SEQ ID NO 4

In some embodiments, the target site region is SEQ ID NO 5

In some embodiments, the target site region is SEQ ID NO 6

In some embodiments, the target site region is SEQ ID NO 7

In some embodiments, the target site region is SEQ ID NO 8

In some embodiments, the target site region is SEQ ID NO 9

In some embodiments, the target site region is SEQ ID NO 10

In some embodiments, the target site region is SEQ ID NO 11

In some embodiments, the target site region is SEQ ID NO 12

In some embodiments, the target site region is SEQ ID NO 13

In some embodiments, the target site region is SEQ ID NO 14

In some embodiments, the target site region is SEQ ID NO 15

In some embodiments, the target site region is SEQ ID NO 16

In some embodiments, the target site region is SEQ ID NO 17

In some embodiments, the target site region is SEQ ID NO 18

In some embodiments, the target site region is SEQ ID NO 19

In some embodiments, the target site region is SEQ ID NO 20

In some embodiments, the target site region is SEQ ID NO 21

In some embodiments, the target site region is SEQ ID NO 22

In some embodiments, the target site region is SEQ ID NO 23

In some embodiments, the target site region is SEQ ID NO 24

In some embodiments, the target site region is SEQ ID NO 25

In some embodiments, the target site region is SEQ ID NO 26

In some embodiments, the target site region is SEQ ID NO 27

In some embodiments, the target site region is SEQ ID NO 28

In some embodiments, the target site region is SEQ ID NO 29

In some embodiments, the target site region is SEQ ID NO 30

In some embodiments, the target site region is SEQ ID NO 31

In some embodiments, the target site region is SEQ ID NO 32

In some embodiments, the target site region is SEQ ID NO 33

In some embodiments, the target site region is SEQ ID NO 34

In some embodiments, the target site region is SEQ ID NO 35

In some embodiments, the target site region is SEQ ID NO 36

In some embodiments, the target site region is SEQ ID NO 37

In some embodiments, the target site region is SEQ ID NO 38

In some embodiments, the target site region is SEQ ID NO 39

In some embodiments, the target site region is SEQ ID NO 40

In some embodiments, the target site region is SEQ ID NO 41

In some embodiments, the target site region is SEQ ID NO 42

In some embodiments, the target site region is SEQ ID NO 43

In some embodiments, the target site region is SEQ ID NO 44

In some embodiments, the target site region is SEQ ID NO 45

In some embodiments, the target site region is SEQ ID NO 46

In some embodiments, the target site region is SEQ ID NO 47

In some embodiments, the target site region is SEQ ID NO 48

In some embodiments, the target site region is SEQ ID NO 49

In some embodiments, the target site region is SEQ ID NO 50

In some embodiments, the target site region is SEQ ID NO 51

In some embodiments, the target site region is SEQ ID NO 52

In some embodiments, the target site region is SEQ ID NO 53

In a further aspect, the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to an exon region of SEQ ID NO 1: The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to a region of SEQ ID NO 1, selected from the group consisting of 81-256; 12486-12582; 17807-17905; 19343-19397; 19570-19602; 20839-20926; 24032-24107; 31667-31775; 32162-32257; 32369-32453; 33167-33290; 34170-34312; 35816-38457.

In a further aspect, the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to an intron region of SEQ ID NO 1 : the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to a region of SEQ ID NO 1, selected from the group consisting of 256-12486; 12582-17807; 17905-19343; 19397-19570; 19602-20839; 20926-24032; 24107-31667; 31775-32162; 32257-32369; 32453-33167; 33290-34170; and 34312-35816.

Target Cell

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell. In some embodiments the target cell is a neuronal cell, such as a brain cell. In some embodiments, the target cell is a motor neuron, such as an upper or lower motor neuron. In some embodiments, the target cell is a cortical neuron. It will be understood that for in vitro assessments for the capability of inhibiting the expression of TIA1, the target cell may be a in vitro primary cell or an in vitro cell culture. For in vivo use, such as in therapy, the target cell is suitably in vivo.

In preferred embodiments the target cell expresses TIA1 mRNA, such as the TIA1 pre-mRNA, e.g. SEQ ID NO 1, or TIA1 mature mRNA (for exon targeting compounds). The poly A tail of TIA1 mRNA is typically disregarded for antisense oligonucleotide targeting.

Naturally Occurring Variant

The term “naturally occurring variant” refers to variants of TIA1 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

The homo sapiens TIA1 gene is located at Chromosome 2: 70,209,444-70,248,660 reverse strand (GRCh38:CM000664.2).

In some embodiments, the naturally occurring variants have at least 95% such as at least 98% or at least 99% homology to a mammalian TIA1 target nucleic acid, such as a target nucleic acid selected form the group consisting of SEQ ID NO 1. In some embodiments the naturally occurring variants have at least 99% homology to the human TIA1 target nucleic acid of SEQ ID NO 1.

Modulation of Expression

The term “modulation of expression” as used herein is to be understood as an overall term for an oligonucleotide's ability to alter the amount of TIA1 protein or TIA1 mRNA when compared to the amount of TIA1 or TIA1 mRNA prior to administration of the oligonucleotide. Alternatively modulation of expression may be determined by reference to a control experiment. It is generally understood that the control is an individual or target cell treated with a e.g. saline composition (no oligonucleotide) or an individual or target cell treated with a non-targeting oligonucleotide (mock).

One preferred type of modulation is an oligonucleotide's ability to inhibit, down-regulate, reduce, suppress, remove, stop, block, prevent, lessen, lower, avoid or terminate expression of TIA1, e.g. by degradation of TIA1 mRNA.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

2′ Sugar Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

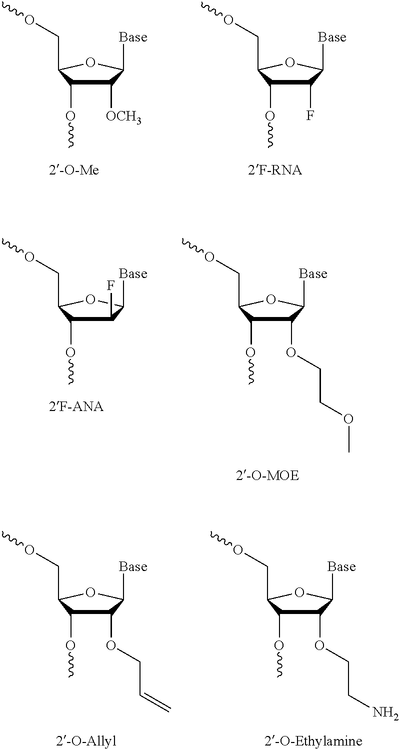

In relation to the present invention 2′ substituted does not include 2′ bridged molecules like LNA.

Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352 , WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

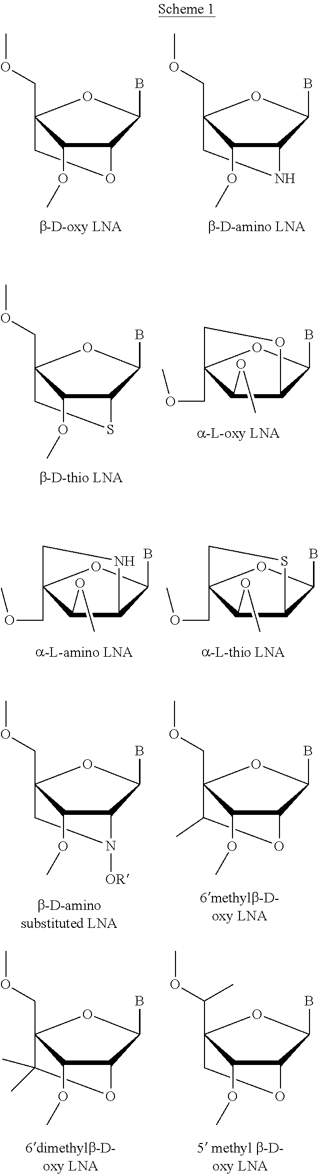

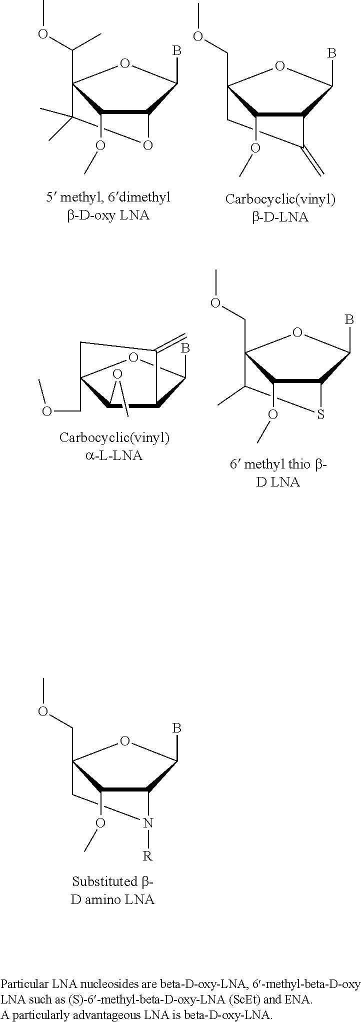

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of

WO01/23613 (hereby incorporated by reference). For use in determining RHase H activity, recombinant human RNase H1 is available from Lubio Science GmbH, Lucerne, Switzerland.

Gapmer

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank. Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to17, such as 16 to18 nucleosides. By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

Regions F, G and F′ are further defined below and can be incorporated into the F-G-F′ formula.

Gapmer—Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. One or more cytosine (C) DNA in the gap region may in some instances be methylated (e.g. when a DNA c is followed by a DNA g) such residues are either annotated as 5-methyl-cytosine (^{me}C). In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages. Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G—“Gap-breaker”

Alternatively, there are numerous reports of the insertion of a modified nucleoside which confers a 3′ endo conformation into the gap region of gapmers, whilst retaining some RNaseH activity.

Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′ —O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region. Exemplary designs for gap-breaker oligonucleotides include

F₁₋₈-[D₃₋₄-E₁-D₃₋₄]-F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

Alternating Flank Gapmers

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and there is at least one DNA nucleoside positioned between the 5′ and 3′ most LNA nucleosides of region F or F′ (or both region F and F′).

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence or gapmer region F-G-F′ (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. DNA phosphodiester containing biocleavable linkers are described in more detail in WO 2014/076195 (hereby incorporated by reference)—see also region D′ or D″ herein.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

The invention relates to oligonucleotides, such as antisense oligonucleotides, targeting TIA1 expression.

The oligonucleotides of the invention targeting TIA1 are capable of hybridizing to and inhibiting the expression of a TIA1 target nucleic acid in a cell which is expressing the TIA1 target nucleic acid.

The TIA1 target nucleic acid may be a mammalian TIA1 mRNA or premRNA, such as a human TIA1 mRNA or premRNA, for example a premRNA or mRNA originating from the Homo sapiens T cell-restricted intracellular antigen-1 (TIA1), RefSeqGene on Chromosome 2: 70,209,444-70,248,660 reverse strand (GRCh38:CM000664.2)—see also Ensembl ENSG00000116001 (SEQ ID NO 1).

The oligonucleotides of the invention are capable of inhibiting the expression of TIA1 target nucleic acid, such as the TIA1 mRNA, in a cell which is expressing the target nucleic acid, such as the TIA1 mRNA.

In some embodiments, the oligonucleotides of the invention are capable of inhibiting the expression of TIA1 target nucleic acid in a cell which is expressing the target nucleic acid, so to reduce the level of TIA1 target nucleic acid (e.g. the mRNA) by at least 50%, at least 60%, at least 70%, at least 80%, or at least 90% inhibition compared to the expression level of the TIA1 target nucleic acid (e.g. the mRNA) in the cell. Example 1 provides a suitable assay for evaluating the ability of the oligonucleotides of the invention to inhibit the expression of the target nucleic acid. Suitably the evaluation of a compounds ability to inhibit the expression of the target nucleic acid is performed in vitro, such a gymnotic in vitro assay, for example as according to Example 1.

An aspect of the present invention relates to an antisense oligonucleotide, such as an LNA antisense oligonucleotide gapmer which comprises a contiguous nucleotide sequence of 10 to 30 nucleotides in length with at least 90% complementarity, such as is fully complementary to SEQ ID NO 1, and/or a sequence selected from the group consisting of SEQ ID NO 4-53.

In some embodiments, the oligonucleotide comprises a contiguous sequence of 10-30 nucleotides, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

In some embodiments, the oligonucleotide of the invention comprises a contiguous nucleotides sequence of 12-24, such as 13, 14, 15, 16, 17, 18, 19, 20, 21, 22, or 23, contiguous nucleotides in length, wherein the contiguous nucleotide sequence is fully complementary to SEQ ID NO 1.

In some embodiments, the antisense oligonucleotide of the invention or the contiguous nucleotide sequence thereof is a gapmer, such as an LNA gapmer, a mixed wing gapmer, or an alternating flank gapmer.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to SEQ ID NO 1.

In some embodiments the contiguous nucleotide sequence of the antisense oligonucleotide according to the invention is less than 20 nucleotides in length. In some embodiments the contiguous nucleotide sequence of the antisense oligonucleotide according to the invention is 12-24 nucleotides in length. In some embodiments the contiguous nucleotide sequence of the antisense oligonucleotide according to the invention is 12-22 nucleotides in length. In some embodiments the contiguous nucleotide sequence of the antisense oligonucleotide according to the invention is 12-20 nucleotides in length. In some embodiments the contiguous nucleotide sequence of the antisense oligonucleotide according to the invention is 12-18 nucleotides in length. In some embodiments the contiguous nucleotide sequence of the antisense oligonucleotide according to the invention is 12-16 nucleotides in length.

Advantageously, in some embodiments all of the internucleoside linkages between the nucleosides of the contiguous nucleotide sequence are phosphorothioate internucleoside linkages.

In some embodiments, the contiguous nucleotide sequence is fully complementary to SEQ ID NO 1.

In some embodiments, the contiguous nucleotide sequence is fully complementary to a sequence selected from the group consisting of SEQ ID NO 4-53.

In some embodiments, the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.

In some embodiments, the sugar modified nucleosides of region F and F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

In some embodiments, region G comprises 5-16 contiguous DNA nucleosides.

In some embodiments, wherein the antisense oligonucleotide is a gapmer oligonucleotide, such as an LNA gapmer oligonucleotide.

In some embodiments, the LNA nucleosides are beta-D-oxy LNA nucleosides.

In some embodiments, the internucleoside linkages between the contiguous nucleotide sequence are phosphorothioate internucleoside linkages.

Exemplary Sequence Motifs and Motif Sequences of the Invention are listed in the table below—see also table A in the examples.:

| Target | Target region | MOTIF SEQ | |
|---|---|---|---|
| region | sequence | ID | Motif Sequence |
| SEQ ID NO 4 | TGTGTTTTATATGAGAAGG | SEQ ID NO 54 | CCTTCTCATATAAAACACA |
| SEQ ID NO 5 | AGGGAGTGTAGTAAAG | SEQ ID NO 55 | CTTTACTACACTCCCT |
| SEQ ID NO 6 | GAAATTTTAAGAATTAGTGG | SEQ ID NO 56 | CCACTAATTCTTAAAATTTC |
| SEQ ID NO 7 | TTGAGAAGTAATTGTTGG | SEQ ID NO 57 | CCAACAATTACTTCTCAA |
| SEQ ID NO 8 | GATGAGGTTGTAAATCAG | SEQ ID NO 58 | CTGATTTACAACCTCATC |
| SEQ ID NO 9 | GGAATTTTGGAGAAAAATA | SEQ ID NO 59 | TATTTTTCTCCAAAATTCC |
| SEQ ID NO 10 | TTATTTGTTGGATGAATGAG | SEQ ID NO 60 | CTCATTCATCCAACAAATAA |
| SEQ ID NO 11 | GGTTTTAGGATGTTTTAGTG | SEQ ID NO 61 | CACTAAAACATCCTAAAACC |
| SEQ ID NO 12 | TTAAAGAGTAAAGAATGGAA | SEQ ID NO 62 | TTCCATTCTTTACTCTTTAA |
| SEQ ID NO 13 | GATTAGGTAGAATATAGTGT | SEQ ID NO 63 | ACACTATATTCTACCTAATC |
| SEQ ID NO 14 | AAATTTTTTAATGGGAAAGG | SEQ ID NO 64 | CCTTTCCCATTAAAAAATTT |
| SEQ ID NO 15 | GTAATGTTAAATGGAAGGT | SEQ ID NO 65 | ACCTTCCATTTAACATTAC |
| SEQ ID NO 16 | GTGTTTGTTGAATGGTAGAT | SEQ ID NO 66 | ATCTACCATTCAACAAACAC |
| SEQ ID NO 17 | AGGAAGATTAAGTTACA | SEQ ID NO 67 | TGTAACTTAATCTTCCT |
| SEQ ID NO 18 | ATAATAATAAGGTTAGGATG | SEQ ID NO 68 | CATCCTAACCTTATTATTAT |
| SEQ ID NO 19 | TAAATAGGAATGTTAGGG | SEQ ID NO 69 | CCCTAACATTCCTATTTA |
| SEQ ID NO 20 | TAAAGATTAGATTGAAGG | SEQ ID NO 70 | CCTTCAATCTAATCTTTA |
| SEQ ID NO 21 | TGAGGAGTATTCAAGGT | SEQ ID NO 71 | ACCTTGAATACTCCTCA |
| SEQ ID NO 22 | ATTTGGGAGGTAGTGAA | SEQ ID NO 72 | TTCACTACCTCCCAAAT |
| SEQ ID NO 23 | GTGATTATTGTGTGTGAGAT | SEQ ID NO 73 | ATCTCACACACAATAATCAC |
| SEQ ID NO 24 | AGTGATTATTGTGTGTGAG | SEQ ID NO 74 | CTCACACACAATAATCACT |
| SEQ ID NO 25 | TTGTATGTAAAGGAATATAT | SEQ ID NO 75 | ATATATTCCTTTACATACAA |
| SEQ ID NO 26 | GTTGTATGTAAAGGAATATA | SEQ ID NO 76 | TATATTCCTTTACATACAAC |
| SEQ ID NO 27 | AGTTGTATGTAAAGGAATAT | SEQ ID NO 77 | ATATTCCTTTACATACAACT |
| SEQ ID NO 28 | AAGTTGTATGTAAAGGAATA | SEQ ID NO 78 | TATTCCTTTACATACAACTT |
| SEQ ID NO 29 | AAAGTTGTATGTAAAGGAAT | SEQ ID NO 79 | ATTCCTTTACATACAACTTT |
| SEQ ID NO 30 | GTGGATAAATGTTGGC | SEQ ID NO 80 | GCCAACATTTATCCAC |
| SEQ ID NO 31 | AGTGGATAAATGTTGG | SEQ ID NO 81 | CCAACATTTATCCACT |
| SEQ ID NO 32 | TGAGGTATGGAGTTTTAG | SEQ ID NO 82 | CTAAAACTCCATACCTCA |
| SEQ ID NO 33 | TGGTGTAATGTCTGGG | SEQ ID NO 83 | CCCAGACATTACACCA |
| SEQ ID NO 34 | GAATGGTGTAATGTCTGG | SEQ ID NO 84 | CCAGACATTACACCATTC |
| SEQ ID NO 35 | TGAATGGTGTAATGTCT | SEQ ID NO 85 | AGACATTACACCATTCA |
| SEQ ID NO 36 | TGAAGGGATTACTGTTT | SEQ ID NO 86 | AAACAGTAATCCCTTCA |
| SEQ ID NO 37 | AGTGAAGGGATTACTGT | SEQ ID NO 87 | ACAGTAATCCCTTCACT |
| SEQ ID NO 38 | AAGTGAAGGGATTACTG | SEQ ID NO 88 | CAGTAATCCCTTCACTT |
| SEQ ID NO 39 | TAAAGTGAAGGGATTACT | SEQ ID NO 89 | AGTAATCCCTTCACTTTA |
| SEQ ID NO 40 | ATATAAAGTGAAGGGATTA | SEQ ID NO 90 | TAATCCCTTCACTTTATAT |
| SEQ ID NO 41 | TGAATGTGTTTGTGTTAATA | SEQ ID NO 91 | TATTAACACAAACACATTCA |
| SEQ ID NO 42 | ATGATTGAATGTGTTTGTGT | SEQ ID NO 92 | ACACAAACACATTCAATCAT |
| SEQ ID NO 43 | TATGATTGAATGTGTTTGTG | SEQ ID NO 93 | CACAAACACATTCAATCATA |
| SEQ ID NO 44 | ATATGATTGAATGTGTTTGT | SEQ ID NO 94 | ACAAACACATTCAATCATAT |
| SEQ ID NO 45 | GATATGATTGAATGTGTTTG | SEQ ID NO 95 | CAAACACATTCAATCATATC |
| SEQ ID NO 46 | AGATTAGGATTTGTCA | SEQ ID NO 96 | TGACAAATCCTAATCT |
| SEQ ID NO 47 | GATAATGGGTAAGGTAA | SEQ ID NO 97 | TTACCTTACCCATTATC |
| SEQ ID NO 48 | AAGATAATGGGTAAGGTA | SEQ ID NO 98 | TACCTTACCCATTATCTT |
| SEQ ID NO 49 | TATGGATGTAAGGGTA | SEQ ID NO 99 | TACCCTTACATCCATA |
| SEQ ID NO 50 | TTATGGATGTAAGGGTATTT | SEQ ID NO 100 | AAATACCCTTACATCCATAA |
| SEQ ID NO 51 | ATTATGGATGTAAGGGT | SEQ ID NO 101 | ACCCTTACATCCATAAT |
| SEQ ID NO 52 | ATGATTATGGATGTAAGG | SEQ ID NO 102 | CCTTACATCCATAATCAT |
| SEQ ID NO 53 | AAATGATTATGGATGTAAG | SEQ ID NO 103 | CTTACATCCATAATCATTT |

The invention provides antisense oligonucleotides according to the invention, such as antisense oligonucleotides 12-24, such as 14-18 in length, nucleosides in length wherein the antisense oligonucleotide comprises a contiguous nucleotide sequence comprising at least 12, such as at least 14, such as at least 15 contiguous nucleotides present in a sequence selected from the group consisting of SEQ ID NO 54-103.

The invention provides an LNA gapmer according to the invention comprising or consisting of a contiguous nucleotide sequence selected from SEQ ID NO SEQ ID NO 54-103.

The invention provides an antisense oligonucleotide selected from the group consisting of: CCttctcatataaaaCACA; CTTtactacactccCT; CCACtaattcttaaaattTC; CCaacaattacttcTCAA; CTGatttacaacctcATC; TATttttctccaaaattCC; CTCAttcatccaacaaatAA; CACtaaaacatcctaaaaCC; TTCCattctttactctttAA; ACActatattctacctaATC; CCtttcccattaaaaaATTT; ACCTtccatttaacattAC; ATCtaccattcaacaaaCAC; TGTaacttaatcttCCT; CAtcctaaccttattatTAT; CCctaacattcctatTTA; CCttcaatctaatcTTTA; ACcttgaatactccTCA; TTCActacctcccaaAT; ATCtcacacacaataatCAC; CTCAcacacaataatcaCT; ATAtattcctttacataCAA; TATAttcctttacatacaAC; ATattcctttacatacaACT; TATTcctttacatacaacTT; ATtcctttacatacaaCTTT; GCCaacatttatccAC; CCAacatttatccACT; CTaaaactccataccTCA; CCcagacattacacCA; CCagacattacaccaTTC; AGAcattacaccatTCA; AAacagtaatcccTTCA; ACAgtaatcccttcaCT; CAGtaatcccttcacTT; AGtaatcccttcacttTA; TAatcccttcactttaTAT; TATTaacacaaacacattCA; ACAcaaacacattcaatCAT; CACAaacacattcaatcaTA; ACAaacacattcaatcaTAT; CAaacacattcaatcaTATC; TGAcaaatcctaaTCT; TTAccttacccattaTC; TAccttacccattatcTT; TACccttacatccATA; AAAtacccttacatccaTAA; ACccttacatccaTAAT; CCTtacatccataatcAT; and CTTAcatccataatcatTT; wherein a capital letter is a LNA nucleoside, and a lower case letter is a DNA nucleoside. In some embodiments all internucleoside linkages in contiguous nucleoside sequence are phosphorothioate internucleoside linkages. Optionally LNA cytosine may be 5-methyl cytosine. Optionally DNA cytosine may be 5-methyl cytosine.

The invention provides an antisense oligonucleotide selected from the group consisting of: CCttctcatataaaaCACA; CTTtactacactccCT; CCACtaattcttaaaattTC; CCaacaattacttcTCAA; CTGatttacaacctcATC; TATttttctccaaaattCC; CTCAttcatccaacaaatAA; CACtaaaacatcctaaaaCC; TTCCattctttactctttAA; ACActatattctacctaATC; CCtttcccattaaaaaATTT; ACCTtccatttaacattAC; ATCtaccattcaacaaaCAC; TGTaacttaatcttCCT; CAtcctaaccttattatTAT; CCctaacattcctatTTA; CCttcaatctaatcTTTA; ACcttgaatactccTCA; TTCActacctcccaaAT; ATCtcacacacaataatCAC; CTCAcacacaataatcaCT; ATAtattcctttacataCAA; TATAttcctttacatacaAC; ATattcctttacatacaACT; TATTcctttacatacaacTT; ATtcctttacatacaaCTTT; GCCaacatttatccAC; CCAacatttatccACT; CTaaaactccataccTCA; CCcagacattacacCA; CCagacattacaccaTTC; AGAcattacaccatTCA; AAacagtaatcccTTCA; ACAgtaatcccttcaCT; CAGtaatcccttcacTT; AGtaatcccttcacttTA; TAatcccttcactttaTAT; TATTaacacaaacacattCA; ACAcaaacacattcaatCAT; CACAaacacattcaatcaTA; ACAaacacattcaatcaTAT; CAaacacattcaatcaTATC; TGAcaaatcctaaTCT; TTAccttacccattaTC; TAccttacccattatcTT; TACccttacatccATA; AAAtacccttacatccaTAA; ACccttacatccaTAAT; CCTtacatccataatcAT; and CTTAcatccataatcatTT; wherein a capital letter is a beta-D-oxy-LNA nucleoside, and a lower case letter is a DNA nucleoside, wherein all internucleoside linkages in the oligonucleotide are phosphorothioate internucleoside linkages, and all LNA cytosines are 5-methyl cytosine.

Further Advantageous Target Site Regions

The invention provides antisense oligonucleotides according to the invention, such as antisense oligonucleotides 12-24, such as 12-18 in length, nucleosides in length wherein the antisense oligonucleotide comprises a contiguous nucleotide sequence comprising at least 14, such as at least 15 contiguous nucleotides, which are fully complementary to a target site region selected from the group consisting of the target sequence regions in LIST A.

The invention provides antisense oligonucleotides according to the invention, such as antisense oligonucleotides 12-24, such as 12-18 in length, nucleosides in length wherein the antisense oligonucleotide comprises a contiguous nucleotide sequence comprising at least 14, such as at least 15 contiguous nucleotides, which are fully complementary to a target site region selected from the group consisting of the target sequence regions in LIST B.

The antisense oligonucleotides according to the invention which target target sequence regions is LIST A or LIST B may be gapmer oligonucleotides, such as LNA gapmer oligonucleotides.

Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO 2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration.

The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

Applications

The oligonucleotides of the invention may be utilized as research reagents for, for example, diagnostics, therapeutics and prophylaxis.

In research, such oligonucleotides may be used to specifically modulate the synthesis of TIA1 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating TIA1 expression in a target cell which is expressing TIA1, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal. In diagnostics the oligonucleotides may be used to detect and quantitate TIA1 expression in cell and tissues by northern blotting, in-situ hybridisation or similar techniques.

For therapeutics, an animal or a human, suspected of having a disease or disorder, which can be treated by modulating the expression of TIA1

The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

In some embodiments, the disease or disorder is a neurodegenerative disease, such as a neurodegenerative disease

In some embodiments, the disease is selected from the group consisting of Amyotrophic Lateral Sclerosis (ALS), Frontotemporal Dementia (FTD), a tauopathy, such as a primary tauopathy, frontotemporal dementia with parkinsonism (FTDP-17), frontotemporal lobar dementia (FTLD-TDP), Huntington's disease, Creutzfeld-Jacob disease, and spinomuscular atrophy, motor neuron disease, Alzheimer's disease, and Welander distal myopathy.

In some embodiments, the disease is Amyotrophic Lateral Sclerosis.

In some embodiments, the disease is Frontotemporal Dementia (FTD).

The invention provides for the oligonucleotide, conjugate or the pharmaceutical composition of the invention for use in medicine.

The disease or disorder, as referred to herein, is associated with expression of TIA1. In some embodiments disease or disorder may be associated with a mutation in the TIA1 gene. Therefore, in some embodiments, the target nucleic acid is a mutated form of the TIA1 sequence.

The methods of the invention are preferably employed for treatment or prophylaxis against diseases caused by abnormal levels and/or activity of TIA1.

The invention further relates to use of an oligonucleotide, oligonucleotide conjugate or a pharmaceutical composition as defined herein for the manufacture of a medicament for the treatment of abnormal levels and/or activity of TIA 1.

Administration

The oligonucleotides or pharmaceutical compositions of the present invention may be administered topical or enteral or parenteral (such as, intravenous, subcutaneous, intra-muscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration.

In some embodiments the active oligonucleotide or oligonucleotide conjugate is administered intrathecally. In some embodiments the active oligonucleotide or oligonucleotide conjugate is administered intracerebroventricularly. In some embodiments the active oligonucleotide or oligonucleotide conjugate is administered intracerebrally.

In some embodiments, the oligonucleotide, oligonucleotide conjugate or pharmaceutical composition of the invention is administered ata dose of 0.1-15 mg/kg, such as from 0.2-10 mg/kg, such as from 0.25-5 mg/kg. The administration can be once a week, every 2^{nd} week, every third week or even once a month.

Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

### EXAMPLES

Compounds and sequence—see Exemplary Sequence Motifs and Compounds of the Invention are listed in table A

Cell Lines

TABLE B
Details for different cell lines used in in vitro screening of TIA1 antisense oligonucleotides.

| Cell lines | | | | | | |
|---|---|---|---|---|---|---|
| | | | Cell medium | | Hours of | |
| | | | (All medium and | | incubation | |
| | | | additives are | | of cells | |
| | | | purchased from | Cells/well | prior to | Days of |
| Name | Vendor | Cat.no. | Sigma Aldrich) | (96 well plate) | treatment | treatment |
| A431 | ECACC | 85090402 | EMEM (Cat.no. | 8000 | 24 | 3 |
| | | | M2279), 10% | | | |
| | | | FBS (Cat.no. | | | |
| | | | F7524), 2 mM | | | |
| | | | Glutamine | | | |
| | | | (Cat.no. G8541), | | | |
| | | | 0.1 mM NEAA | | | |
| | | | (Cat.no. M7145), | | | |
| | | | 25 μg/ml | | | |
| | | | Gentamicin | | | |
| | | | (Cat.no. G1397) | | | |
| NCI-H23 | ATCC | CRL-5800 | RPMI 1640 | 10000 | 24 | 3 |
| | | | (Cat.no. R2405), | | | |
| | | | 10% FBS | | | |
| | | | (Cat.no. F7524), | | | |
| | | | 10 mM Hepes | | | |
| | | | (Cat.no. H0887), | | | |
| | | | 1 mM Sodium | | | |
| | | | Pyruvate | | | |
| | | | (Cat.no. S8636), | | | |
| | | | 25 μg/ml | | | |
| | | | Gentamicin | | | |
| | | | (Cat.no. G1397) | | | |
| ARPE19 | ATCC | CRL-2302 | DMEM/F-12 | 2000 | 0 | 4 |
| | | | HAM (Cat.no. | | | |
| | | | D8437), 10% | | | |
| | | | FBS (Cat.no. | | | |
| | | | F7524), 25 μg/ml | | | |
| | | | Gentamicin | | | |
| | | | (Cat.no. G1397) | | | |
| U251 | ECACC | 9063001 | EMEM (Cat.no. | 2000 | 0 | 4 |
| | | | M2279), 10% | | | |
| | | | FBS (Cat.no. | | | |
| | | | F7524), 2 mM | | | |
| | | | Glutamine | | | |
| | | | (Cat.no. G8541), | | | |
| | | | 0.1 mM NEAA | | | |
| | | | (Cat.no. M7145) | | | |
| | | | 1 mM Sodium | | | |
| | | | Pyruvate | | | |
| | | | (Cat.no. S8636), | | | |
| | | | 25 μg/ml | | | |
| | | | Gentamicin | | | |
| | | | (Cat.no. G1397) | | | |
| U2O5 | ATCC | HTB-96 | MCCoy 5A | 7000 | 24 | 3 |
| | | | medium (Cat.no. | | | |
| | | | M8403), 10% | | | |
| | | | FBS (Cat.no. | | | |
| | | | F7524), 1.5 mM | | | |
| | | | Glutamine | | | |
| | | | (Cat.no. G8541), | | | |
| | | | 25 μg/ml | | | |
| | | | Gentamicin | | | |
| | | | (Cat.no. G1397) | | | |

Compounds may also be evaluated in iPSC-derived motor neurons.

### Example 1: Testing In Vitro Efficacy of LNA Oligonucleotides of the Compounds Listed in Table A in U2OS Cell Line at 25 and 5 μM

An oligonucleotide screen was done in the human cell line, U2OS, using the 50 LNA oligonucleotides listed in table X. The U2OS cell line was purchased from ATCC (cat. no.: HTB-96) and maintained as recommended by the supplier in a humidified incubator at 37° C. with 5% CO₂. For the screening assays, cells were seeded in 96 multi well plates in media recommended by the supplier (MCCoy 5A medium [Cat.no. M8403], 10% FBS [Cat.no. F7524], 1.5mM Glutamine [Cat.no. G8541], 25 μg/ml Gentamicin [Cat.no. G1397]). The number of cells/well has been optimized to 7000 cells/well in a 96 well format

Cells were incubated 24 hours before addition of the oligonucleotide in concentrations of 5 or 25 μM (dissolved in PBS). 3 days after addition of the oligonucleotide, the cells were harvested. RNA was extracted using the Qiagen RNeasy 96 kit (74182), according to the manufacturer's instructions). cDNA synthesis and qPCR was performed using qScript XLT one-step RT-qPCR ToughMix Low ROX, 95134-100 (Quanta Biosciences). Target transcript levels were quantified using FAM labeled TaqMan assays from Thermo Fisher Scientific in a multiplex reaction with a VIC labelled GAPDH control. TaqMan primer assays for the target transcript of interest TIA1 (Hs00234977_m1 (FAM-MGB)), and a house keeping gene GAPDH (4326317E VIC-MGB probe). A technical duplex set up was used, n=2 biological independent replicates.

The relative TIA1 mRNA expression levels are shown in Table X as % of control (PBS-treated cells) i.e. the lower the value the larger the inhibition.

TABLE C
in vitro efficacy of anti-TIA1 compounds (average
of 2 biological independent with stdev). TIA1
mRNA levels are normalized experiments
to GAPDH and shown as % of control (PBS
treated cells).

| | U2OS screening | |
|---|---|---|
| | 3 days gymnosis | |
| | TIA1/GAPDH | |
| | RICC-18-5324 | |
| | n = 2, 7000 cells/well | |
| Compound ID # | 25 μM | 5 μM |
| COMP# 54 | 17 | 25 |
| COMP# 55 | 9 | 20 |
| COMP# 56 | 104 | 95 |
| COMP# 57 | 36 | 57 |
| COMP# 58 | 0.8 | 3 |
| COMP# 59 | 57 | 72 |
| COMP# 60 | 32 | 50 |
| COMP# 61 | 99 | 90 |
| COMP# 62 | 11 | 20 |
| COMP# 63 | 27 | 45 |
| COMP# 64 | 78 | 85 |
| COMP# 65 | 32 | 43 |
| COMP# 66 | 71 | 82 |
| COMP# 67 | 26 | 47 |
| COMP# 68 | 113 | 94 |
| COMP# 69 | 101 | 103 |
| COMP# 70 | 73 | 88 |
| COMP# 71 | 4 | 11 |
| COMP# 72 | 20 | 47 |
| COMP# 73 | 60 | 95 |
| COMP# 74 | 48 | 67 |
| COMP# 75 | 69 | 75 |
| COMP# 76 | 48 | 74 |
| COMP# 77 | 41 | 68 |
| COMP# 78 | 15 | 30 |
| COMP# 79 | 25 | 38 |
| COMP# 80 | 11 | 25 |
| COMP# 81 | 4 | 12 |
| COMP# 82 | 15 | 36 |
| COMP# 83 | 2 | 8 |
| COMP# 84 | 0.6 | 2 |
| COMP# 85 | 0.3 | 0.8 |
| COMP# 86 | 6 | 19 |
| COMP# 87 | 10 | 24 |
| COMP# 88 | 20 | 38 |
| COMP# 89 | 57 | 72 |
| COMP# 90 | 77 | 82 |
| COMP# 91 | 42 | 69 |
| COMP# 92 | 57 | 76 |
| COMP# 93 | 41 | 60 |
| COMP# 94 | 84 | 87 |
| COMP# 95 | 79 | 94 |
| COMP# 96 | 90 | 92 |
| COMP# 97 | 81 | 93 |
| COMP# 98 | 88 | 93 |
| COMP# 99 | 82 | 89 |
| COMP# 100 | 95 | 96 |
| COMP# 101 | 89 | 95 |
| COMP# 102 | 96 | 91 |
| COMP# 103 | 96 | 105 |

Exemplary Sequence Motifs and Compounds of the Invention are listed in table A:

TABLE A

| Target | Target region | | | Compound | |
|---|---|---|---|---|---|
| region | sequence | MOTIF SEQ ID | Motif Sequence | ID NO # | Compound |
| SEQ ID NO 4 | TGTGTTTTATATGAGAAGG | SEQ ID NO 54 | CCTTCTCATATAAAACACA | #54 | CCttctcatataaaaCACA |
| SEQ ID NO 5 | AGGGAGTGTAGTAAAG | SEQ ID NO 55 | CTTTACTACACTCCCT | #55 | CTTtactacactccCT |
| SEQ ID NO 6 | GAAATTTTAAGAATTAGTGG | SEQ ID NO 56 | CCACTAATTCTTAAAATTTC | #56 | CCACtaattcttaaaattTC |
| SEQ ID NO 7 | TTGAGAAGTAATTGTTGG | SEQ ID NO 57 | CCAACAATTACTTCTCAA | #57 | CCaacaattacttcTCAA |
| SEQ ID NO 8 | GATGAGGTTGTAAATCAG | SEQ ID NO 58 | CTGATTTACAACCTCATC | #58 | CTGatttacaacctcATC |
| SEQ ID NO 9 | GGAATTTTGGAGAAAAATA | SEQ ID NO 59 | TATTTTTCTCCAAAATTCC | #59 | TATffitctccaaaattCC |
| SEQ ID NO 10 | TTATTTGTTGGATGAATGAG | SEQ ID NO 60 | CTCATTCATCCAACAAATAA | #60 | CTCAttcatccaacaaatAA |
| SEQ ID NO 11 | GGTTTTAGGATGTTTTAGTG | SEQ ID NO 61 | CACTAAAACATCCTAAAACC | #61 | CACtaaaacatcctaaaaCC |
| SEQ ID NO 12 | TTAAAGAGTAAAGAATGGAA | SEQ ID NO 62 | TTCCATTCTTTACTCTTTAA | #62 | TTCCattctttactctttAA |
| SEQ ID NO 13 | GATTAGGTAGAATATAGTGT | SEQ ID NO 63 | ACACTATATTCTACCTAATC | #63 | ACActatattctacctaATC |
| SEQ ID NO 14 | AAATTTTTTAATGGGAAAGG | SEQ ID NO 64 | CCTTTCCCATTAAAAAATTT | #64 | CCtttcccattaaaaaATTT |
| SEQ ID NO 15 | GTAATGTTAAATGGAAGGT | SEQ ID NO 65 | ACCTTCCATTTAACATTAC | #65 | ACCTtccatttaacattAC |
| SEQ ID NO 16 | GTGTTTGTTGAATGGTAGAT | SEQ ID NO 66 | ATCTACCATTCAACAAACAC | #66 | ATCtaccattcaacaaaCAC |
| SEQ ID NO 17 | AGGAAGATTAAGTTACA | SEQ ID NO 67 | TGTAACTTAATCTTCCT | #67 | TGTaacttaatcttCCT |
| SEQ ID NO 18 | ATAATAATAAGGTTAGGATG | SEQ ID NO 68 | CATCCTAACCTTATTATTAT | #68 | CAtcctaaccttattatTAT |
| SEQ ID NO 19 | TAAATAGGAATGTTAGGG | SEQ ID NO 69 | CCCTAACATTCCTATTTA | #69 | CCctaacattcctatTTA |
| SEQ ID NO 20 | TAAAGATTAGATTGAAGG | SEQ ID NO 70 | CCTTCAATCTAATCTTTA | #70 | CCttcaatctaatcTTTA |
| SEQ ID NO 21 | TGAGGAGTATTCAAGGT | SEQ ID NO 71 | ACCTTGAATACTCCTCA | #71 | ACcttgaatactccTCA |
| SEQ ID NO 22 | ATTTGGGAGGTAGTGAA | SEQ ID NO 72 | TTCACTACCTCCCAAAT | #72 | TTCActacctcccaaAT |
| SEQ ID NO 23 | GTGATTATTGTGTGTGAGAT | SEQ ID NO 73 | ATCTCACACACAATAATCAC | #73 | ATCtcacacacaataatCAC |
| SEQ ID NO 24 | AGTGATTATTGTGTGTGAG | SEQ ID NO 74 | CTCACACACAATAATCACT | #74 | CTCAcacacaataatcaCT |
| SEQ ID NO 25 | TTGTATGTAAAGGAATATAT | SEQ ID NO 75 | ATATATTCCTTTACATACAA | #75 | ATAtattcctttacataCAA |
| SEQ ID NO 26 | GTTGTATGTAAAGGAATATA | SEQ ID NO 76 | TATATTCCTTTACATACAAC | #76 | TATAttcctttacatacaAC |
| SEQ ID NO 27 | AGTTGTATGTAAAGGAATAT | SEQ ID NO 77 | ATATTCCTTTACATACAACT | #77 | ATattcctttacatacaACT |
| SEQ ID NO 28 | AAGTTGTATGTAAAGGAATA | SEQ ID NO 78 | TATTCCTTTACATACAACTT | #78 | TATTcctttacatacaacTT |
| SEQ ID NO 29 | AAAGTTGTATGTAAAGGAAT | SEQ ID NO 79 | ATTCCTTTACATACAACTTT | #79 | ATtcctttacatacaaCTTT |
| SEQ ID NO 30 | GTGGATAAATGTTGGC | SEQ ID NO 80 | GCCAACATTTATCCAC | #80 | GCCaacatttatccAC |
| SEQ ID NO 31 | AGTGGATAAATGTTGG | SEQ ID NO 81 | CCAACATTTATCCACT | #81 | CCAacatttatccACT |
| SEQ ID NO 32 | TGAGGTATGGAGTTTTAG | SEQ ID NO 82 | CTAAAACTCCATACCTCA | #82 | CTaaaactccataccTCA |
| SEQ ID NO 33 | TGGTGTAATGTCTGGG | SEQ ID NO 83 | CCCAGACATTACACCA | #83 | CCcagacattacacCA |
| SEQ ID NO 34 | GAATGGTGTAATGTCTGG | SEQ ID NO 84 | CCAGACATTACACCATTC | #84 | CCagacattacaccaTTC |
| SEQ ID NO 35 | TGAATGGTGTAATGTCT | SEQ ID NO 85 | AGACATTACACCATTCA | #85 | AGAcattacaccatTCA |
| SEQ ID NO 36 | TGAAGGGATTACTGTTT | SEQ ID NO 86 | AAACAGTAATCCCTTCA | #86 | AAacagtaatcccTTCA |
| SEQ ID NO 37 | AGTGAAGGGATTACTGT | SEQ ID NO 87 | ACAGTAATCCCTTCACT | #87 | ACAgtaatcccttcaCT |
| SEQ ID NO 38 | AAGTGAAGGGATTACTG | SEQ ID NO 88 | CAGTAATCCCTTCACTT | #88 | CAGtaatcccttcacTT |
| SEQ ID NO 39 | TAAAGTGAAGGGATTACT | SEQ ID NO 89 | AGTAATCCCTTCACTTTA | #89 | AGtaatcccttcacttTA |
| SEQ ID NO 40 | ATATAAAGTGAAGGGATTA | SEQ ID NO 90 | TAATCCCTTCACTTTATAT | #90 | TAatcccttcactttaTAT |
| SEQ ID NO 41 | TGAATGTGTTTGTGTTAATA | SEQ ID NO 91 | TATTAACACAAACACATTCA | #91 | TATTaacacaaacacattCA |
| SEQ ID NO 42 | ATGATTGAATGTGTTTGTGT | SEQ ID NO 92 | ACACAAACACATTCAATCAT | #92 | ACAcaaacacattcaatCAT |
| SEQ ID NO 43 | TATGATTGAATGTGTTTGTG | SEQ ID NO 93 | CACAAACACATTCAATCATA | #93 | CACAaacacattcaatcaTA |
| SEQ ID NO 44 | ATATGATTGAATGTGTTTGT | SEQ ID NO 94 | ACAAACACATTCAATCATAT | #94 | ACAaacacattcaatcaTAT |
| SEQ ID NO 45 | GATATGATTGAATGTGTTTG | SEQ ID NO 95 | CAAACACATTCAATCATATC | #95 | CAaacacattcaatcaTATC |
| SEQ ID NO 46 | AGATTAGGATTTGTCA | SEQ ID NO 96 | TGACAAATCCTAATCT | #96 | TGAcaaatcctaaTCT |
| SEQ ID NO 47 | GATAATGGGTAAGGTAA | SEQ ID NO 97 | TTACCTTACCCATTATC | #97 | TTAccttacccattaTC |
| SEQ ID NO 48 | AAGATAATGGGTAAGGTA | SEQ ID NO 98 | TACCTTACCCATTATCTT | #98 | TAccttacccattatcTT |
| SEQ ID NO 49 | TATGGATGTAAGGGTA | SEQ ID NO 99 | TACCCTTACATCCATA | #99 | TACccttacatccATA |
| SEQ ID NO 50 | TTATGGATGTAAGGGTATTT | SEQ ID NO 100 | AAATACCCTTACATCCATAA | #100 | AAAtacccttacatccaTAA |
| SEQ ID NO 51 | ATTATGGATGTAAGGGT | SEQ ID NO 101 | ACCCTTACATCCATAAT | #101 | ACccttacatccaTAAT |
| SEQ ID NO 52 | ATGATTATGGATGTAAGG | SEQ ID NO 102 | CCTTACATCCATAATCAT | #102 | CCTtacatccataatcAT |
| SEQ ID NO 53 | AAATGATTATGGATGTAAG | SEQ ID NO 103 | CTTACATCCATAATCATTT | #103 | CTTAcatccataatcatTT |

In the compound column, capital letters are beta-D-oxy LNA nucleosides, and LNA C are all 5-methyl C, lower case letters are DNA nucleosides, and optionally a superscript m before a lower case c represent a 5-methyl cytosine DNA nucleoside, and all internucleoside linkages are phosphorothioate internucleoside linkages.

1. An antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to SEQ ID NO 1, wherein the antisense oligonucleotide is capable of inhibiting the expression of human TIA1 in a cell which is expressing human TIA1; or a pharmaceutically acceptable salt thereof.

2. The antisense oligonucleotide according to claim 1, wherein the contiguous nucleotide sequence is at least 90% complementary, such as fully complementary to a sequence selected from the group consisting of SEQ ID NO 4-53.

3. The antisense oligonucleotide according to any one of claims 1 to 3, wherein the contiguous nucleotide sequence is fully complementary to a region of SEQ ID NO 1, selected from the group consisting of the regions in list A.

4. The antisense oligonucleotide according to any one of claims 1 to 3, wherein the contiguous nucleotide sequence is fully complementary to a region of SEQ ID NO 1, selected from the group consisting of the regions in list B.

5. The antisense oligonucleotide according to any one of claims 1-4, wherein the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.

6. The antisense oligonucleotide according to claim 5, wherein the sugar modified nucleosides of region F and F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

7. The antisense oligonucleotide according to claim 5 or 6, wherein region G comprises 5-16 contiguous DNA nucleosides.

8. The antisense oligonucleotide according to any one of claims 1-7, wherein the antisense oligonucleotide is a LNA gapmer oligonucleotide.

9. The antisense oligonucleotide according to any one of claims 5-8, wherein the LNA nucleosides are beta-D-oxy LNA nucleosides.

10. The antisense oligonucleotide according to any one of claims 1-9, wherein the internucleoside linkages between the contiguous nucleotide sequence are phosphorothioate internucleoside linkages.

11. The antisense oligonucleotide according to any one of claims 1-10, wherein the oligonucleotide comprises a contiguous nucleotide sequence selected from the group consisting of: 54-103.

12. The antisense oligonucleotide according to any one of claims 1-11, wherein the oligonucleotide comprises or consists of a contiguous nucleotide sequence, selected from the group consisting of: CCttctcatataaaaCACA (SEQ ID NO 54);
CTTtactacactccCT (SEQ ID NO 55); CCACtaattcttaaaattTC (SEQ ID NO 56);
CCaacaattacttcTCAA (SEQ ID NO 57); CTGatttacaacctcATC (SEQ ID NO 58);
TATttttctccaaaattCC (SEQ ID NO 59); CTCAttcatccaacaaatAA (SEQ ID NO 60);
CACtaaaacatcctaaaaCC (SEQ ID NO 61); TTCCattctttactctttAA (SEQ ID NO 62);
ACActatattctacctaATC (SEQ ID NO 63); CCtttcccattaaaaaATTT (SEQ ID NO 64);
ACCTtccatttaacattAC (SEQ ID NO 65); ATCtaccattcaacaaaCAC (SEQ ID NO 66);
TGTaacttaatcttCCT (SEQ ID NO 67); CAtcctaaccttattatTAT (SEQ ID NO 68);
CCctaacattcctatTTA (SEQ ID NO 69); CCttcaatctaatcTTTA (SEQ ID NO 70);
ACcttgaatactccTCA (SEQ ID NO 71); TTCActacctcccaaAT (SEQ ID NO 72);
ATCtcacacacaataatCAC (SEQ ID NO 73); CTCAcacacaataatcaCT (SEQ ID NO 74);
ATAtattcctttacataCAA (SEQ ID NO 75); TATAttcctttacatacaAC (SEQ ID NO 76);
ATattcctttacatacaACT (SEQ ID NO 77); TATTcctttacatacaacTT (SEQ ID NO 78);
ATtcctttacatacaaCTTT (SEQ ID NO 79); GCCaacatttatccAC (SEQ ID NO 80);
CCAacatttatccACT (SEQ ID NO 81); CTaaaactccataccTCA (SEQ ID NO 82);
CCcagacattacacCA (SEQ ID NO 83); CCagacattacaccaTTC (SEQ ID NO 84);
AGAcattacaccatTCA (SEQ ID NO 85); AAacagtaatcccTTCA (SEQ ID NO 86);
ACAgtaatcccttcaCT (SEQ ID NO 87); CAGtaatcccttcacTT (SEQ ID NO 88);
AGtaatcccttcacttTA (SEQ ID NO 89); TAatcccttcactttaTAT (SEQ ID NO 90);
TATTaacacaaacacattCA (SEQ ID NO 91); ACAcaaacacattcaatCAT (SEQ ID NO 92);
CACAaacacattcaatcaTA (SEQ ID NO 93); ACAaacacattcaatcaTAT (SEQ ID NO 94);
CAaacacattcaatcaTATC (SEQ ID NO 95); TGAcaaatcctaaTCT (SEQ ID NO 96);
TTAccttacccattaTC (SEQ ID NO 97); TAccttacccattatcTT (SEQ ID NO 98);
TACccttacatccATA (SEQ ID NO 99); AAAtacccttacatccaTAA (SEQ ID NO 100);
ACccttacatccaTAAT (SEQ ID NO 101); CCTtacatccataatcAT (SEQ ID NO 102); and
CTTAcatccataatcatTT (SEQ ID NO 103), wherein a capital letter represents a LNA nucleoside, a lower case letter represents a DNA nucleoside.

13. The antisense oligonucleotide according to any one of claims 1-12, wherein the oligonucleotide comprises or consists of a contiguous nucleotide sequence, selected from the group consisting of: CCttctcatataaaaCACA (SEQ ID NO 54);
CTTtactacactccCT (SEQ ID NO 55); CCACtaattcttaaaattTC (SEQ ID NO 56);
CCaacaattacttcTCAA (SEQ ID NO 57); CTGatttacaacctcATC (SEQ ID NO 58);
TATttttctccaaaattCC (SEQ ID NO 59); CTCAttcatccaacaaatAA (SEQ ID NO 60);
CACtaaaacatcctaaaaCC (SEQ ID NO 61); TTCCattctttactctttAA (SEQ ID NO 62);
ACActatattctacctaATC (SEQ ID NO 63); CCtttcccattaaaaaATTT (SEQ ID NO 64);
ACCTtccatttaacattAC (SEQ ID NO 65); ATCtaccattcaacaaaCAC (SEQ ID NO 66);
TGTaacttaatcttCCT (SEQ ID NO 67); CAtcctaaccttattatTAT (SEQ ID NO 68);
CCctaacattcctatTTA (SEQ ID NO 69); CCttcaatctaatcTTTA (SEQ ID NO 70);
ACcttgaatactccTCA (SEQ ID NO 71); TTCActacctcccaaAT (SEQ ID NO 72);
ATCtcacacacaataatCAC (SEQ ID NO 73); CTCAcacacaataatcaCT (SEQ ID NO 74);
ATAtattcctttacataCAA (SEQ ID NO 75); TATAttcctttacatacaAC (SEQ ID NO 76);
ATattcctttacatacaACT (SEQ ID NO 77); TATTcctttacatacaacTT (SEQ ID NO 78);
ATtcctttacatacaaCTTT (SEQ ID NO 79); GCCaacatttatccAC (SEQ ID NO 80);
CCAacatttatccACT (SEQ ID NO 81); CTaaaactccataccTCA (SEQ ID NO 82);
CCcagacattacacCA (SEQ ID NO 83); CCagacattacaccaTTC (SEQ ID NO 84);
AGAcattacaccatTCA (SEQ ID NO 85); AAacagtaatcccTTCA (SEQ ID NO 86);
ACAgtaatcccttcaCT (SEQ ID NO 87); CAGtaatcccttcacTT (SEQ ID NO 88);
AGtaatcccttcacttTA (SEQ ID NO 89); TAatcccttcactttaTAT (SEQ ID NO 90);
TATTaacacaaacacattCA (SEQ ID NO 91); ACAcaaacacattcaatCAT (SEQ ID NO 92);
CACAaacacattcaatcaTA (SEQ ID NO 93); ACAaacacattcaatcaTAT (SEQ ID NO 94);
CAaacacattcaatcaTATC (SEQ ID NO 95); TGAcaaatcctaaTCT (SEQ ID NO 96);
TTAccttacccattaTC (SEQ ID NO 97); TAccttacccattatcTT (SEQ ID NO 98);
TACccttacatccATA (SEQ ID NO 99); AAAtacccttacatccaTAA (SEQ ID NO 100);
ACccttacatccaTAAT (SEQ ID NO 101); CCTtacatccataatcAT (SEQ ID NO 102); and
CTTAcatccataatcatTT (SEQ ID NO 103), wherein a capital letter represents a beta-D-oxy LNA nucleoside, a lower case letter represents a DNA nucleoside, wherein each LNA cytosine is 5-methyl cytosine, and wherein the internucleoside linkages between the nucleosides are phosphorothioate internucleoside linkages.

14. A conjugate comprising the oligonucleotide according to any one of claims 1-13, and at least one conjugate moiety covalently attached to said oligonucleotide.

15. A pharmaceutical composition comprising the oligonucleotide of claim 1-13 or the conjugate of claim 14 and a pharmaceutically acceptable diluent, solvent, carrier, salt and/or adjuvant.

16. An in vivo or in vitro method for modulating TIA1 expression in a target cell which is expressing TIA1, said method comprising administering an oligonucleotide of any one of claims 1-13, the conjugate according to claim 14, or the pharmaceutical composition of claim 15 in an effective amount to said cell.

17. A method for treating or preventing a disease comprising administering a therapeutically or prophylactically effective amount of an oligonucleotide of any one of claims 1-13 or the conjugate according to claim 14 or the pharmaceutical composition of claim 15 to a subject suffering from or susceptible to the disease.

18. The method of claim 17, wherein the disease is a neurological disorder, such as a neurological disorder selected from the group consisting of Amyotrophic Lateral Sclerosis (ALS), Frontotemporal Dementia (FTD), tauopathy (such as primary tauopathy), frontotemporal dementia with parkinsonism (FTDP-17), frontotemporal lobar dementia (FTLD-TDP), Huntington's disease, Creutzfeld-Jacob disease, and spinomuscular atrophy, motor neuron disease, Tauopathy, Alzheimer's disease, and Welander distal myopathy.

19. The oligonucleotide of any one of claims 1-13 or the conjugate according to claim 14 or the pharmaceutical composition of claim 15 for use in medicine.

20. The oligonucleotide of any one of claims 1-13 or the conjugate according to claim 14 or the pharmaceutical composition of claim 15 for use in the treatment or prevention of a neurological disorder, such as a neurological disorder selected from the group consisting of Amyotrophic Lateral Sclerosis (ALS), Frontotemporal Dementia (FTD), tauopathy (such as primary tauopathy), frontotemporal dementia with parkinsonism (FTDP-17), frontotemporal lobar dementia (FTLD-TDP), Huntington's disease, Creutzfeld-Jacob disease, and spinomuscular atrophy, motor neuron disease, Tauopathy, Alzheimer's disease, and Welander distal myopathy.

21. Use of the oligonucleotide of claims 1-13 or the conjugate according to claim 14 or the pharmaceutical composition of claim 15, for the preparation of a medicament for treatment or prevention of a neurological disorder, such as a neurological disorder selected from the group consisting of Amyotrophic Lateral Sclerosis (ALS), Frontotemporal Dementia (FTD) , frontotemporal dementia with parkinsonism (FTDP-17), frontotemporal lobar dementia (FTLD-TDP), tauopathy (such as primary tauopathy), Huntington's disease, Creutzfeld-Jacob disease, and spinomuscular atrophy, motor neuron disease, Tauopathy, Alzheimer's disease, and Welander distal myopathy.

22. The Use of method according to any one of claims 17-21 , wherein the neurological disorder is Amyotrophic Lateral Sclerosis (ALS).

23. The Use of method according to any one of claims 17-21, wherein the neurological disorder is a tauopathy, such as a primary tauopathy.

24. The Use of method according to any one of claims 17-21 , wherein the neurological disorder is frontotemporal lobar dementia (FTLD-TDP).
